SEQUENCE LISTING (4 sequences)

SEQ ID NO: 1 - RNA, 21 nt, Artificial Sequence
  Description of Artificial Sequence Synthetic oligonucleotide
aacaguguucuugcucuauaa

SEQ ID NO: 2 - RNA, 23 nt, Artificial Sequence
  Description of Artificial Sequence Synthetic oligonucleotide
uuauagagcaagaacacuguuuu

SEQ ID NO: 3 - RNA, 21 nt, Artificial Sequence
  Description of Artificial Sequence Synthetic oligonucleotide
aacaguguucuugcucuauaa

SEQ ID NO: 4 - RNA, 23 nt, Artificial Sequence
  Description of Artificial Sequence Synthetic oligonucleotide
uuauagagcaagaacacuguuuu

(19) United States
(12) Patent Application Publication
(10) Pub. No.: US 2022/0062323 A1
(43) Pub. Date: Mar. 3, 2022

(54) BIVALENT TARGETED CONJUGATES

(71) Applicants: Richard J. HOLLAND, Burnaby (CA); Lorne Ralph PALMER, Burnaby (CA); Mark WOOD, Burnaby (CA); ARBUTUS BIOPHARMA CORPORATION, Burnaby (CA)

(72) Inventors: James HEYES, Vancouver (CA); Richard J. HOLLAND, Vancouver (CA); Lorne Ralph PALMER, Vancouver (CA); Mark WOOD, Port Moody (CA)

(73) Assignee: ARBUTUS BIOPHARMA CORPORATION, Burnaby, BC (CA)

(21) Appl. No.: 17/290,615

(22) PCT Filed: Nov. 4, 2019
(86) PCT No.: PCT/US2019/059687

§ 371 Date: Apr. 30, 2021

Related U.S. Application Data
(60) Provisional application No. 62/755,179, filed on Nov. 2, 2018.

(51) Int. Cl. A61K 31/7105; A61K 47/54

(52) U.S. Cl. CPC A61K 31/7105; A61K 47/545; A61K 47/549

(57) ABSTRACT

The invention provides conjugates that comprise a bivalent targeting moiety, a nucleic acid, and optional linking groups as well as synthetic intermediates and synthetic methods useful for preparing the conjugates, compositions comprising the bidentate targeting ligands and the conjugates, as well as methods for targeting therapeutic nucleic acids with the bidentate conjugates. The conjugates are useful to target therapeutic nucleic acids.

### CROSS-REFERENCE TO RELATED APPLICATION(S)

This patent application claims the benefit of priority of U.S. application Ser. No. 62/755,179, filed Nov. 2, 2018, which application is herein incorporated by reference.

### BACKGROUND

Since the seminal paper by Ashwell and Morell elucidating the role of the asialoglycoprotein receptor (ASGPr) in the recognition and transport of circulating glycoproteins, this receptor has been the focus of intense research (D'Souza et al, 2015, J. Control Release, 203, 126-139). High levels of expression on the surface of hepatocytes make this receptor an attractive target for liver specific delivery agents. The receptor has a trivalent carbohydrate binding domain that selectively binds N-acetylgalactose amine. The generally accepted rule is that the binding affinity for a targeting ligand increases with the number of GalNac units in the following order: six GalNac units are greater than four GalNac units, which are greater than three GalNac units, which are greater than two GalNac units, which are greater than one GalNac unit (Meier et al, 2000, J Mol Biol, 300, 857-865; Spiess M, 1990, Biochemistry, 29, 43, 10009-10018; Grewal P., 2010, Methods in Enzymology, 479, 223-241; Lee, et al., 1983, J Biol Chem, 258, 1, 199-202; and Valentijn, et al., 1997, Tetrahedron, 53, 2, 759-770).

In most cases, the chemical synthesis of polydentate targeting ligands can be involved, requiring multiple synthetic steps (sometimes between 20-30). This impacts manufacturing requirements and the cost of goods. Moreover, the synthesis of GalNAc/siRNA conjugates is typically carried out on an immobilized controlled pore glass (CPG) support. Access to the reactive sites on the support is related to pore size, and thus is negatively impacted by the size of the molecules accessing the sites. An increase in targeting ligand size (number of monosaccharide units, molecular weight, molecular radius, etc.) negatively impacts loading efficiency on the support. Accordingly, there is currently a need for targeting ligands that have useful delivery properties, but are easier to prepare, less expensive to prepare, have lower molecular weights, and/or have higher loading efficiencies.

### BRIEF SUMMARY

Bidentate GalNac targeting ligands containing two saccharide groups (e.g. N-acetyl galactosamine moieties) with targeting activities as good as or better than known tri-antennary and tetra-antennary ligands have been identified. These bidentate targeting ligands generally have shorter synthetic routes leading to higher total synthesis efficiencies. In addition, their smaller molecule size allows greater penetration onto CPG, resulting in loading levels about 30-50% higher compared to some tri- and tetra-antennary ligands. Additionally, the bidentate targeting ligands have simplified analytics compared to tri- and tetra-antennary ligands, which can expedite ADME-toxicity investigations and related research activities. The invention provides bidentate targeting ligands, nucleic acid conjugates of these bidentate targeting ligands, compositions comprising the bidentate targeting ligands and the conjugates, as well as methods for targeting therapeutic nucleic acids with the bidentate conjugates.

In one embodiment the invention provides a conjugate of formula (I):

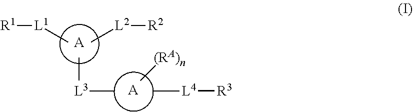

wherein:

R¹ is a saccharide;

L¹ is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

B is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl;

L² is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

R² is a saccharide;

L³ is absent or a linking group;

A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

n is 0, 1, 2, 3, 4, 5, 6, 7, 8, 9, or 10;

L⁴ is absent or a linking group;

R³ is a nucleic acid;

R^{a} is hydrogen, a protecting group, a covalent bond to a solid support, or a bond to a linking group L⁵ that is bound to a solid support; and

L⁵ is a linking group;

or a salt thereof.

The invention also provides a pharmaceutical composition comprising a conjugate of formula I as described herein, or a pharmaceutically acceptable salt thereof, and a pharmaceutically acceptable carrier.

The invention also provides synthetic intermediates and methods disclosed herein that are useful to prepare conjugates of formula I.

Other objects, features, and advantages of the present invention will be apparent to one of skill in the art from the following detailed description and figures.

### DETAILED DESCRIPTION

As used herein, the following terms have the meanings ascribed to them unless specified otherwise.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “animal” includes mammalian species, such as a human, mouse, rat, dog, cat, hamster, guinea pig, rabbit, livestock, and the like.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (e.g., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “cycloalkyl” refers to a saturated or partially unsaturated (non-aromatic) all carbon ring having 3 to 8 carbon atoms (i.e., (C₃-C₈) carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The terms “halo” or “halogen” mean, unless otherwise stated, a fluorine, chlorine, bromine, or iodine atom.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl and furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “saccharide” includes monosaccharides, disaccharides and trisaccharides, all of which can be optionally substituted. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of a compound of formula I through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound of formula I through an ether bond.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-4 (e.g., 1, 2, 3, or 4) modified and/or unmodified deoxythymidine (t or dT) nucleotides, 1-4 (e.g., 1, 2, 3, or 4) modified (e.g., 2′OMe) and/or unmodified uridine (U) ribonucleotides, and/or 1-4 (e.g., 1, 2, 3, or 4) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA of the invention to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 8100, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl, silyl and 2,2-dimethoxy propene. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P.GM. Wuts and T. W. Greene, Greene's Protective Groups in Organic Synthesis 4^{th} edition, Wiley-Interscience, New York, 2006.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP), (1-[Bis(dimethylamino)methylene]-1H-1,2,3-triazolo[4,5-b]pyridinium 3-oxid hexafluorophosphate (HATU), propylphosphonic anhydride solution (T3P) or O-benzotriazol-1-yl-N,N,N′,N′-tetramethyluronium hexafluorophosphate (HBTU).

### Nucleic Acids

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), self-amplifying RNA (saRNA), and combinations thereof. Accordingly, in the context of this invention, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

In certain embodiments a bidentate targeting ligand described herein may be conjugated to a nucleic acid. In certain embodiments, the nucleic acid is a nucleic acid described herein. For example, the nucleic acids used herein can be single-stranded DNA or RNA, or double-stranded DNA or RNA, or DNA-RNA hybrids. Examples of double-stranded RNA are described herein and include, e.g., siRNA and other RNAi agents such as aiRNA and pre-miRNA. Single-stranded nucleic acids include, e.g., antisense oligonucleotides, ribozymes, mature miRNA, and triplex-forming oligonucleotides.

In certain embodiments, the nucleic acid is an oligonucleotide. In particular embodiments, the oligonucleotide ranges from about 10 to about 100 nucleotides in length. In various related embodiments, oligonucleotides, both single-stranded, double-stranded, and triple-stranded, may range in length from about 10 to about 60 nucleotides, from about 15 to about 60 nucleotides, from about 20 to about 50 nucleotides, from about 15 to about 30 nucleotides, or from about 20 to about 30 nucleotides in length.

In certain embodiments, the nucleic acid is selected from the group consisting of small interfering RNA (siRNA), Dicer-substrate dsRNA, small hairpin RNA (shRNA), asymmetrical interfering RNA (aiRNA), microRNA (miRNA), tRNA, rRNA, tRNA, viral RNA (vRNA), self-amplifying RNA (sa-RNA), and combinations thereof.

In certain embodiments, the nucleic acid is an antisense molecule. In certain embodiments, the nucleic acid is a miRNA molecule. In certain embodiments, the nucleic acid is a siRNA. Suitable siRNA, as well as method and intermediates useful for their preparation are reported in International Patent Application Publication Number WO2016/054421.

Target Genes

In certain embodiments, the nucleic acid (e.g., siRNA) may be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses, ligand receptor genes, and genes associated with neurodegenerative disorders. In certain embodiments, the gene of interest is expressed in hepatocytes.

Genes associated with viral infection and survival include those expressed by a virus in order to bind, enter, and replicate in a cell. Of particular interest are viral sequences associated with chronic viral diseases. Viral sequences of particular interest include sequences of Filoviruses such as Ebola virus and Marburg virus (see, e.g., Geisbert et al., J. Infect. Dis., 193:1650-1657 (2006)); Arenaviruses such as Lassa virus, Junin virus, Machupo virus, Guanarito virus, and Sabia virus (Buchmeier et al., Arenaviridae: the viruses and their replication, In: FIELDS VIROLOGY, Knipe et al. (eds.), 4th ed., Lippincott-Raven, Philadelphia, (2001)); Influenza viruses such as Influenza A, B, and C viruses, (see, e.g., Steinhauer et al., Annu Rev Genet., 36:305-332 (2002); and Neumann et al., J Gen Virol., 83:2635-2662 (2002)); Hepatitis viruses (see, e.g., Hamasaki et al., FEBS Lett., 543:51 (2003); Yokota et al., EMBO Rep., 4:602 (2003); Schlomai et al., Hepatology, 37:764 (2003); Wilson et al., Proc. Natl. Acad. Sci. USA, 100:2783 (2003); Kapadia et al., Proc. Natl. Acad. Sci. USA, 100:2014 (2003); and FIELDS VIROLOGY, Knipe et al. (eds.), 4th ed., Lippincott-Raven, Philadelphia (2001)); Human Immunodeficiency Virus (HIV) (Banerjea et al., Mol. Ther., 8:62 (2003); Song et al., J. Virol., 77:7174 (2003); Stephenson, JAMA, 289:1494 (2003); Qin et al., Proc. Natl. Acad. Sci. USA, 100:183 (2003)); Herpes viruses (Jia et al., J. Virol., 77:3301 (2003)); and Human Papilloma Viruses (HPV) (Hall et al., J. Virol., 77:6066 (2003); Jiang et al., Oncogene, 21:6041 (2002)).

Exemplary Filovirus nucleic acid sequences that can be silenced include, but are not limited to, nucleic acid sequences encoding structural proteins (e.g., VP30, VP35, nucleoprotein (NP), polymerase protein (L-pol)) and membrane-associated proteins (e.g., VP40, glycoprotein (GP), VP24). Complete genome sequences for Ebola virus are set forth in, e.g., Genbank Accession Nos. NC_002549; AY769362; NC_006432; NC_004161; AY729654; AY354458; AY142960; AB050936; AF522874; AF499101; AF272001; and AF086833. Ebola virus VP24 sequences are set forth in, e.g., Genbank Accession Nos. U77385 and AY058897. Ebola virus L-pol sequences are set forth in, e.g., Genbank Accession No. X67110. Ebola virus VP40 sequences are set forth in, e.g., Genbank Accession No. AY058896. Ebola virus NP sequences are set forth in, e.g., Genbank Accession No. AY058895. Ebola virus GP sequences are set forth in, e.g., Genbank Accession No. AY058898; Sanchez et al., Virus Res., 29:215-240 (1993); Will et al., J. Virol., 67:1203-1210 (1993); Volchkov et al., FEBS Lett., 305:181-184 (1992); and U.S. Pat. No. 6,713,069. Additional Ebola virus sequences are set forth in, e.g., Genbank Accession Nos. L11365 and X61274. Complete genome sequences for Marburg virus are set forth in, e.g., Genbank Accession Nos. NC_001608; AY430365; AY430366; and AY358025. Marburg virus GP sequences are set forth in, e.g., Genbank Accession Nos. AF005734; AF005733; and AF005732. Marburg virus VP35 sequences are set forth in, e.g., Genbank Accession Nos. AF005731 and AF005730. Additional Marburg virus sequences are set forth in, e.g., Genbank Accession Nos. X64406; Z29337; AF005735; and Z12132. Non-limiting examples of siRNA molecules targeting Ebola virus and Marburg virus nucleic acid sequences include those described in U.S. Patent Publication No. 20070135370, the disclosure of which is herein incorporated by reference in its entirety for all purposes.

Exemplary Influenza virus nucleic acid sequences that can be silenced include, but are not limited to, nucleic acid sequences encoding nucleoprotein (NP), matrix proteins (M1 and M2), nonstructural proteins (NS1 and NS2), RNA polymerase (PA, PB1, PB2), neuraminidase (NA), and haemagglutinin (HA). Influenza A NP sequences are set forth in, e.g., Genbank Accession Nos. NC_004522; AY818138; AB166863; AB188817; AB189046; AB189054; AB189062; AY646169; AY646177; AY651486; AY651493; AY651494; AY651495; AY651496; AY651497; AY651498; AY651499; AY651500; AY651501; AY651502; AY651503; AY651504; AY651505; AY651506; AY651507; AY651509; AY651528; AY770996; AY790308; AY818138; and AY818140. Influenza A PA sequences are set forth in, e.g., Genbank Accession Nos. AY818132; AY790280; AY646171; AY818132; AY818133; AY646179; AY818134; AY551934; AY651613; AY651610; AY651620; AY651617; AY651600; AY651611; AY651606; AY651618; AY651608; AY651607; AY651605; AY651609; AY651615; AY651616; AY651640; AY651614; AY651612; AY651621; AY651619; AY770995; and AY724786. Non-limiting examples of siRNA molecules targeting Influenza virus nucleic acid sequences include those described in U.S. Patent Publication No. 20070218122, the disclosure of which is herein incorporated by reference in its entirety for all purposes.

Exemplary hepatitis virus nucleic acid sequences that can be silenced include, but are not limited to, nucleic acid sequences involved in transcription and translation (e.g., En1, En2, X, P) and nucleic acid sequences encoding structural proteins (e.g., core proteins including C and C-related proteins, capsid and envelope proteins including S, M, and/or L proteins, or fragments thereof) (see, e.g., FIELDS VIROLOGY, supra). Exemplary Hepatitis C virus (HCV) nucleic acid sequences that can be silenced include, but are not limited to, the 5′-untranslated region (5′-UTR), the 3′-untranslated region (3′-UTR), the polyprotein translation initiation codon region, the internal ribosome entry site (IRES) sequence, and/or nucleic acid sequences encoding the core protein, the E1 protein, the E2 protein, the p7 protein, the NS2 protein, the NS3 protease/helicase, the NS4A protein, the NS4B protein, the NS5A protein, and/or the NS5B RNA-dependent RNA polymerase. HCV genome sequences are set forth in, e.g., Genbank Accession Nos. NC_004102 (HCV genotype 1a), AJ238799 (HCV genotype 1b), NC_009823 (HCV genotype 2), NC_009824 (HCV genotype 3), NC_009825 (HCV genotype 4), NC_009826 (HCV genotype 5), and NC_009827 (HCV genotype 6). Hepatitis A virus nucleic acid sequences are set forth in, e.g., Genbank Accession No. NC_001489; Hepatitis B virus nucleic acid sequences are set forth in, e.g., Genbank Accession No. NC_003977; Hepatitis D virus nucleic acid sequence are set forth in, e.g., Genbank Accession No. NC_001653; Hepatitis E virus nucleic acid sequences are set forth in, e.g., Genbank Accession No. NC_001434; and Hepatitis G virus nucleic acid sequences are set forth in, e.g., Genbank Accession No. NC_001710. Silencing of sequences that encode genes associated with viral infection and survival can conveniently be used in combination with the administration of conventional agents used to treat the viral condition. Non-limiting examples of siRNA molecules targeting hepatitis virus nucleic acid sequences include those described in U.S. Patent Publication Nos. 20060281175, 20050058982, and 20070149470; U.S. Pat. No. 7,348,314; and U.S. Provisional Application No. 61/162,127, filed Mar. 20, 2009, the disclosures of which are herein incorporated by reference in their entirety for all purposes.

Genes associated with metabolic diseases and disorders (e.g., disorders in which the liver is the target and liver diseases and disorders) include, for example, genes expressed in dyslipidemia (e.g., liver X receptors such as LXRα and LXRβ (Genback Accession No. NM_007121), farnesoid X receptors (FXR) (Genbank Accession No. NM_005123), sterol-regulatory element binding protein (SREBP), site-1 protease (SIP), 3-hydroxy-3-methylglutaryl coenzyme-A reductase (HMG coenzyme-A reductase), apolipoprotein B (ApoB) (Genbank Accession No. NM_000384), apolipoprotein CIII (ApoC3) (Genbank Accession Nos. NM_000040 and NG_008949 REGION: 5001.8164), and apolipoprotein E (ApoE) (Genbank Accession Nos. NM_000041 and NG_007084 REGION: 5001.8612)); and diabetes (e.g., glucose 6-phosphatase) (see, e.g., Forman et al., Cell, 81:687 (1995); Seol et al., Mol. Endocrinol., 9:72 (1995), Zavacki et al., Proc. Natl. Acad. Sci. USA, 94:7909 (1997); Sakai et al., Cell, 85:1037-1046 (1996); Duncan et al., J. Biol. Chem., 272:12778-12785 (1997); Willy et al., Genes Dev., 9:1033-1045 (1995); Lehmann et al., J. Biol. Chem., 272:3137-3140 (1997); Janowski et al., Nature, 383:728-731 (1996); and Peet et al., Cell, 93:693-704 (1998)). One of skill in the art will appreciate that genes associated with metabolic diseases and disorders (e.g., diseases and disorders in which the liver is a target and liver diseases and disorders) include genes that are expressed in the liver itself as well as and genes expressed in other organs and tissues. Silencing of sequences that encode genes associated with metabolic diseases and disorders can conveniently be used in combination with the administration of conventional agents used to treat the disease or disorder. Non-limiting examples of siRNA molecules targeting the ApoB gene include those described in U.S. Patent Publication No. 20060134189, the disclosure of which is herein incorporated by reference in its entirety for all purposes. Non-limiting examples of siRNA molecules targeting the ApoC3 gene include those described in U.S. Provisional Application No. 61/147,235, filed Jan. 26, 2009, the disclosure of which is herein incorporated by reference in its entirety for all purposes.

Examples of gene sequences associated with tumorigenesis and cell transformation (e.g., cancer or other neoplasia) include mitotic kinesins such as Eg5 (KSP, KIF11; Genbank Accession No. NM_004523); serine/threonine kinases such as polo-like kinase 1 (PLK-1) (Genbank Accession No. NM_005030; Barr et al., Nat. Rev. Mol. Cell. Biol., 5:429-440 (2004)); tyrosine kinases such as WEE1 (Genbank Accession Nos. NM_003390 and NM_001143976); inhibitors of apoptosis such as XIAP (Genbank Accession No. NM_001167); COP9 signalosome subunits such as CSN1, CSN2, CSN3, CSN4, CSN5 (JAB1; Genbank Accession No. NM_006837); CSN6, CSN7A, CSN7B, and CSN8; ubiquitin ligases such as COP1 (RFWD2; Genbank Accession Nos. NM_022457 and NM_001001740); and histone deacetylases such as HDAC1, HDAC2 (Genbank Accession No. NM_001527), HDAC3, HDAC4, HDAC5, HDAC6, HDAC7, HDAC8, HDAC9, etc. Non-limiting examples of siRNA molecules targeting the Eg5 and XIAP genes include those described in U.S. patent application Ser. No. 11/807,872, filed May 29, 2007, the disclosure of which is herein incorporated by reference in its entirety for all purposes. Non-limiting examples of siRNA molecules targeting the PLK-1 gene include those described in U.S. Patent Publication Nos. 20050107316 and 20070265438; and U.S. patent application Ser. No. 12/343,342, filed Dec. 23, 2008, the disclosures of which are herein incorporated by reference in their entirety for all purposes. Non-limiting examples of siRNA molecules targeting the CSN5 gene include those described in U.S. Provisional Application No. 61/045,251, filed Apr. 15, 2008, the disclosure of which is herein incorporated by reference in its entirety for all purposes.

Additional examples of gene sequences associated with tumorigenesis and cell transformation include translocation sequences such as MLL fusion genes, BCR-ABL (Wilda et al., Oncogene, 21:5716 (2002); Scherr et al., Blood, 101:1566 (2003)), TEL-AML1, EWS-FLI1, TLS-FUS, PAX3-FKHR, BCL-2, AML1-ETO, and AML1-MTG8 (Heidenreich et al., Blood, 101:3157 (2003)); overexpressed sequences such as multidrug resistance genes (Nieth et al., FEBS Lett., 545:144 (2003); Wu et al, Cancer Res. 63:1515 (2003)), cyclins (Li et al., Cancer Res., 63:3593 (2003); Zou et al., Genes Dev., 16:2923 (2002)), beta-catenin (Verma et al., Clin Cancer Res., 9:1291 (2003)), telomerase genes (Kosciolek et al., Mol Cancer Ther., 2:209 (2003)), c-MYC, N-MYC, BCL-2, growth factor receptors (e.g., EGFR/ErbB1 (Genbank Accession Nos. NM_005228, NM_201282, NM_201283, and NM_201284; see also, Nagy et al. Exp. Cell Res., 285:39-49 (2003), ErbB2/HER-2 (Genbank Accession Nos. NM_004448 and NM_001005862), ErbB3 (Genbank Accession Nos. NM_001982 and NM_001005915), and ErbB4 (Genbank Accession Nos. NM_005235 and NM_001042599); and mutated sequences such as RAS (reviewed in Tuschl and Borkhardt, Mol. Interventions, 2:158 (2002)). Non-limiting examples of siRNA molecules targeting the EGFR gene include those described in U.S. patent application Ser. No. 11/807,872, filed May 29, 2007, the disclosure of which is herein incorporated by reference in its entirety for all purposes.

Silencing of sequences that encode DNA repair enzymes find use in combination with the administration of chemotherapeutic agents (Collis et al., Cancer Res., 63:1550 (2003)). Genes encoding proteins associated with tumor migration are also target sequences of interest, for example, integrins, selectins, and metalloproteinases. The foregoing examples are not exclusive. Those of skill in the art will understand that any whole or partial gene sequence that facilitates or promotes tumorigenesis or cell transformation, tumor growth, or tumor migration can be included as a template sequence.

Angiogenic genes are able to promote the formation of new vessels. Of particular interest is vascular endothelial growth factor (VEGF) (Reich et al., Mol. Vis., 9:210 (2003)) or VEGFR. siRNA sequences that target VEGFR are set forth in, e.g., GB 2396864; U.S. Patent Publication No. 20040142895; and CA 2456444, the disclosures of which are herein incorporated by reference in their entirety for all purposes.

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see, e.g., U.S. Pat. No. 6,174,861), angiostatin (see, e.g., U U.S. Pat. No. 5,639,725), and VEGFR2 (see, e.g., Decaussin et al., J. Pathol., 188: 369-377 (1999)), the disclosures of which are herein incorporated by reference in their entirety for all purposes. Immunomodulator genes are genes that modulate one or more immune responses. Examples of immunomodulator genes include, without limitation, cytokines such as growth factors (e.g., TGF-α, TGF-β, EGF, FGF, IGF, NGF, PDGF, CGF, GM-CSF, SCF, etc.), interleukins (e.g., IL-2, IL-4, IL-12 (Hill et al., J. Immunol., 171:691 (2003)), IL-15, IL-18, IL-20, etc.), interferons (e.g., IFN-α, IFN-β, IFN-γ, etc.) and TNF. Fas and Fas ligand genes are also immunomodulator target sequences of interest (Song et al., Nat. Med., 9:347 (2003)). Genes encoding secondary signaling molecules in hematopoietic and lymphoid cells are also included in the present invention, for example, Tec family kinases such as Bruton's tyrosine kinase (Btk) (Heinonen et al., FEBS Lett., 527:274 (2002)).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc. Templates coding for an expansion of trinucleotide repeats (e.g., CAG repeats) find use in silencing pathogenic sequences in neurodegenerative disorders caused by the expansion of trinucleotide repeats, such as spinobulbular muscular atrophy and Huntington's Disease (Caplen et al., Hum. Mol. Genet., 11:175 (2002)).

Certain other target genes, which may be targeted by a nucleic acid (e.g., by siRNA) to downregulate or silence the expression of the gene, include but are not limited to, Actin, Alpha 2, Smooth Muscle, Aorta (ACTA2), Alcohol dehydrogenase 1A (ADH1A), Alcohol dehydrogenase 4 (ADH4), Alcohol dehydrogenase 6 (ADH6), Afamin (AFM), Angiotensinogen (AGT), Serine-pyruvate aminotransferase (AGXT), Alpha-2-HS-glycoprotein (AHSG), Aldo-keto reductase family 1 member C4 (AKR1C4), Serum albumin (ALB), alpha-1-microglobulin/bikunin precursor (AMBP), Angiopoietin-related protein 3 (ANGPTL3), Serum amyloid P-component (APCS), Apolipoprotein A-II (APOA2), Apolipoprotein B-100 (APOB), Apolipoprotein C3 (APOC3), Apolipoprotein C-IV (APOC4), Apolipoprotein F (APOF), Beta-2-glycoprotein 1 (APOH), Aquaporin-9 (AQP9), Bile acid-CoA:amino acid N-acyltransferase (BAAT), C4b-binding protein beta chain (C4BPB), Putative uncharacterized protein encoded by LINC01554 (C5orf27), Complement factor 3 (C3), Complement Factor 5 (C5), Complement component C6 (C6), Complement component C8 alpha chain (C8A), Complement component C8 beta chain (C8B), Complement component C8 gamma chain (C8G), Complement component C9 (C9), Calmodulin Binding Transcription Activator 1 (CAMTA1), CD38 (CD38), Complement Factor B (CFB), Complement factor H-related protein 1 (CFHR1), Complement factor H-related protein 2 (CFHR2), Complement factor H-related protein 3 (CFHR3), Cannabinoid receptor 1 (CNR1), ceruloplasmin (CP), carboxypeptidase B2 (CPB2), Connective tissue growth factor (CTGF), C—X—C motif chemokine 2 (CXCL2), Cytochrome P450 1A2 (CYP1A2), Cytochrome P450 2A6 (CYP2A6), Cytochrome P450 2C8 (CYP2C8), Cytochrome P450 2C9 (CYP2C9), Cytochrome P450 Family 2 Subfamily D Member 6 (CYP2D6), Cytochrome P450 2E1 (CYP2E1), Phylloquinone omega-hydroxylase CYP4F2 (CYP4F2), 7-alpha-hydroxycholest-4-en-3-one 12-alpha-hydroxylase (CYP8B1), Dipeptidyl peptidase 4 (DPP4), coagulation factor 12 (F12), coagulation factor II (thrombin) (F2), coagulation factor IX (F9), fibrinogen alpha chain (FGA), fibrinogen beta chain (FGB), fibrinogen gamma chain (FGG), fibrinogen-like 1 (FGL1), flavin containing monooxygenase 3 (FMO3), flavin containing monooxygenase 5 (FMO5), group-specific component (vitamin D binding protein) (GC), Growth hormone receptor (GHR), glycine N-methyltransferase (GNMT), hyaluronan binding protein 2 (HABP2), hepcidin antimicrobial peptide (HAMP), hydroxyacid oxidase (glycolate oxidase) 1 (HAO1), HGF activator (HGFAC), haptoglobin-related protein; haptoglobin (HPR), hemopexin (HPX), histidine-rich glycoprotein (HRG), hydroxysteroid (11-beta) dehydrogenase 1 (HSD11B1), hydroxysteroid (17-beta) dehydrogenase 13 (HSD17B13), Inter-alpha-trypsin inhibitor heavy chain H1 (ITIH1), Inter-alpha-trypsin inhibitor heavy chain H2 (ITIH2), Inter-alpha-trypsin inhibitor heavy chain H3 (ITIH3), Inter-alpha-trypsin inhibitor heavy chain H4 (ITIH4), Prekallikrein (KLKB1), Lactate dehydrogenase A (LDHA), liver expressed antimicrobial peptide 2 (LEAP2), leukocyte cell-derived chemotaxin 2 (LECT2), Lipoprotein (a) (LPA), mannan-binding lectin serine peptidase 2 (MASP2), S-adenosylmethionine synthase isoform type-1 (MAT1A), NADPH Oxidase 4 (NOX4), Poly [ADP-ribose] polymerase 1 (PARP1), paraoxonase 1 (PON1), paraoxonase 3 (PON3), Vitamin K-dependent protein C (PROC), Retinol dehydrogenase 16 (RDH16), serum amyloid A4, constitutive (SAA4), serine dehydratase (SDS), Serpin Family A Member 1 (SERPINA1), Serpin A11 (SERPINA11), Kallistatin (SERPINA4), Corticosteroid-binding globulin (SERPINA6), Antithrombin-III (SERPINC1), Heparin cofactor 2 (SERPIND1), Serpin Family H Member 1 (SERPINH1), Solute Carrier Family 5 Member 2 (SLC5A2), Sodium/bile acid cotransporter (SLC10A1), Solute carrier family 13 member 5 (SLC13A5), Solute carrier family 22 member 1 (SLC22A1), Solute carrier family 25 member 47 (SLC25A47), Solute carrier family 2, facilitated glucose transporter member 2 (SLC2A2), Sodium-coupled neutral amino acid transporter 4 (SLC38A4), Solute carrier organic anion transporter family member 1B1 (SLCO1B1), Sphingomyelin Phosphodiesterase 1 (SMPD1), Bile salt sulfotransferase (SULT2A1), tyrosine aminotransferase (TAT), tryptophan 2,3-dioxygenase (TDO2), UDP glucuronosyltransferase 2 family, polypeptide B10 (UGT2B10), UDP glucuronosyltransferase 2 family, polypeptide B15 (UGT2B15), UDP glucuronosyltransferase 2 family, polypeptide B4 (UGT2B4) and vitronectin (VTN).

In addition to its utility in silencing the expression of any of the above-described genes for therapeutic purposes, certain nucleic acid (e.g., siRNA) described herein are also useful in research and development applications as well as diagnostic, prophylactic, prognostic, clinical, and other healthcare applications. As a non-limiting example, certain nucleic acid (e.g., siRNA) can be used in target validation studies directed at testing whether a gene of interest has the potential to be a therapeutic target. Certain nucleic acid (e.g., siRNA) can also be used in target identification studies aimed at discovering genes as potential therapeutic targets.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope of this invention. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Linking Group

The compounds and conjugates of the invention may include one or more linking groups (e.g. L³ or L⁴). The structure of each linking group can vary, provided the conjugate functions as described herein. For example, the structure of each linking group vary in length and atom composition, and each linking group can be branched, non-branched, cyclic, or a combination thereof. The linking group may also modulate the solubility, stability, or aggregation properties of the conjugate.

In one embodiment each linking group comprises about 3-1000 atoms. In one embodiment each linking group comprises about 3-500 atoms. In one embodiment each linking group comprises about 3-200 atoms. In one embodiment each linking group comprises about 3-50 atoms. In one embodiment each linking group comprises about 10-1000 atoms. In one embodiment each linking group comprises about 10-500 atoms. In one embodiment each linking group comprises about 10-200 atoms. In one embodiment each linking group comprises about 10-50 atoms.

In one embodiment each linking group comprises atoms selected from H, C, N, S and O.

In one embodiment each linking group comprises atoms selected from H, C, N, S, P and O.

In one embodiment each linking group comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl. In one embodiment the linker comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, wherein each R^{a} is independently H or (C₁-C₆)alkyl.

In one embodiment each linking group comprises a polyethylene glycol. In one embodiment the linking group comprises a polyethylene glycol linked to the remainder of the targeted conjugate by a carbonyl group. In one embodiment the polyethylene glycol comprises about 1 to about 500 or about 5 to about 500 or about 3 to about 100 repeat (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

### EMBODIMENTS OF THE INVENTION

One aspect of the invention is a compound of formula I, as set forth about in the Summary of the Invention, or a salt thereof.

In one embodiment A is absent.

In one embodiment A is a 3-20 membered cycloalkyl, a 5-20 membered aryl, a 5-20 membered heteroaryl, or a 3-20 membered heterocycloalkyl.

In one embodiment B is a 5-10 membered aryl.

In one embodiment B is naphthyl or phenyl.

In one embodiment B is phenyl.

In one embodiment the group:

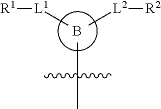

is:

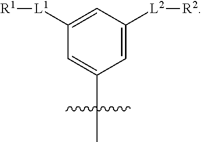

In one embodiment B is a 5-10 membered heteroaryl.

In one embodiment B is pyridyl, pyrimidyl, quinolyl, isoquinolyl, imidazoyl, thiazolyl, oxadiazolyl or oxazolyl.

In one embodiment the group:

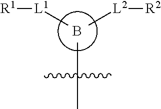

is:

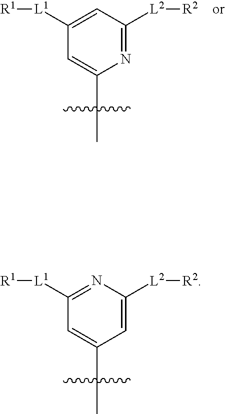

In one embodiment the group:

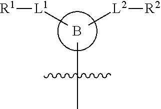

is:

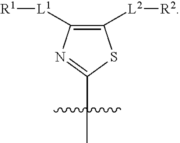

In one embodiment L¹ is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In one embodiment L¹ is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In one embodiment L¹ is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In one embodiment L² is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In one embodiment L² is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In one embodiment L² is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In one embodiment R¹ is:

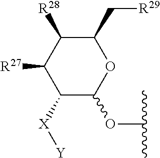

wherein:

X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent

R²⁰ is hydrogen or (C₁-C₄)alkyl;

R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;

R²⁷ is —OH, —NR²⁵R²⁶ or —F;

R²⁸ is —OH, —NR²⁵R²⁶ or —F;

R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;

each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;

each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and

each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In one embodiment R¹ is:

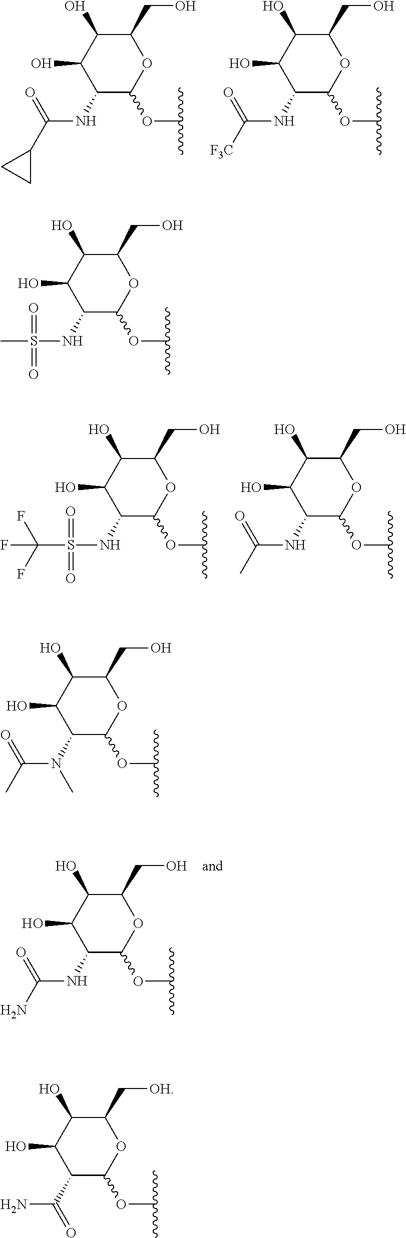

In one embodiment R¹ is:

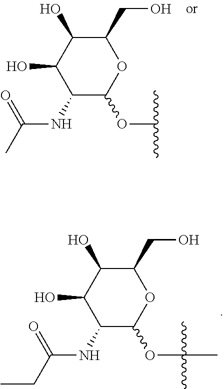 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment R¹ is:

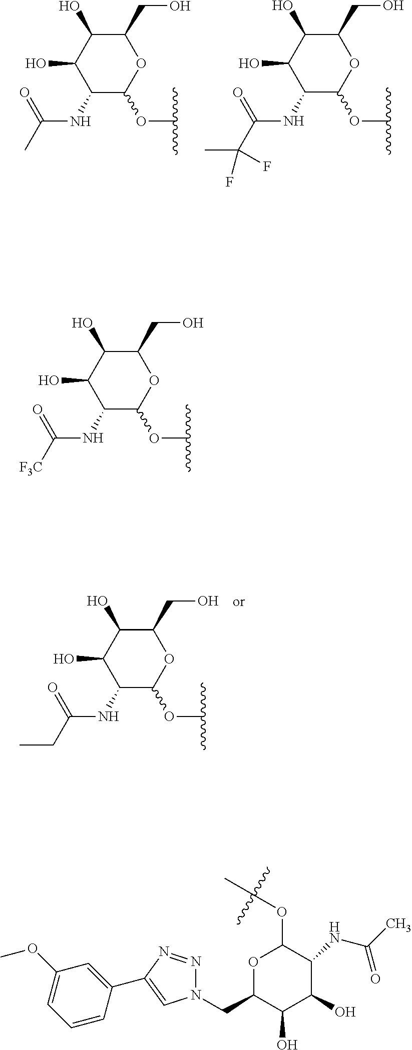

In one embodiment R¹ is:

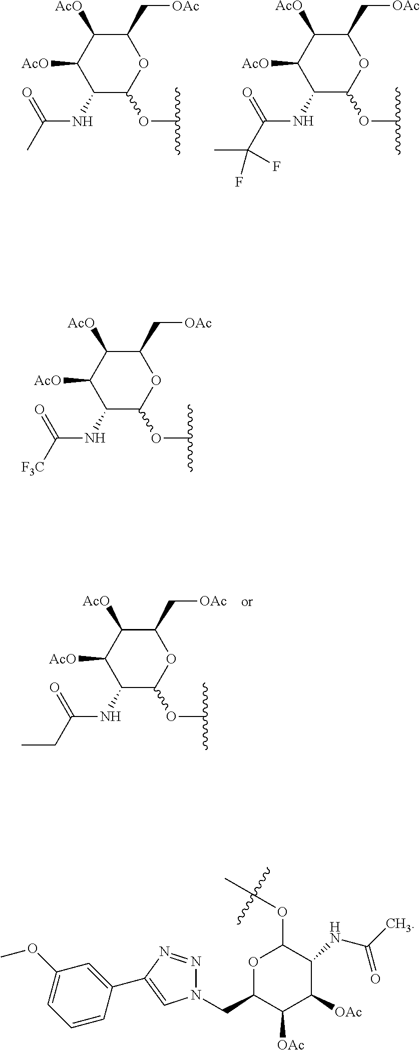 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1O[C@H](Cn2cc(-c3cccc(OC)c3)nn2)[C@H](OC(C)=O)[C@H](OC(C)=O)[C@H]1NC(C)=O]

In one embodiment R² is:

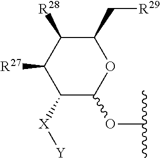

wherein:

X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent

R²⁰ is hydrogen or (C₁-C₄)alkyl;

R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₅)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;

R²⁷ is —OH, —NR²⁵R²⁶ or —F;

R²⁸ is —OH, —NR²⁵R²⁶ or —F;

R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;

each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;

each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and

each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In one embodiment R² is:

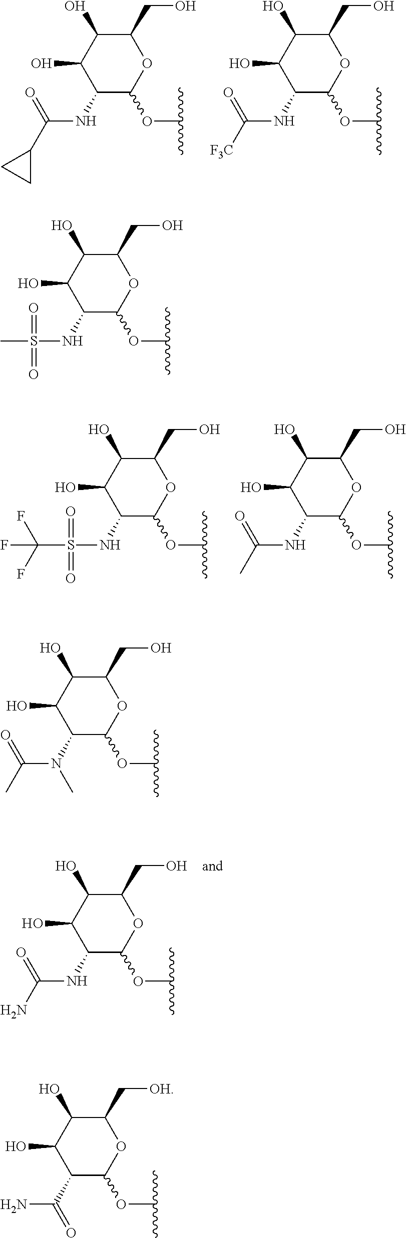

In one embodiment R² is:

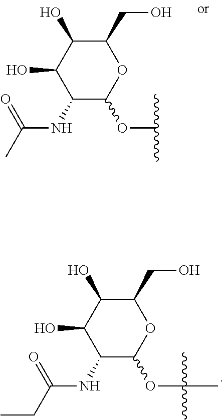 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment R² is:

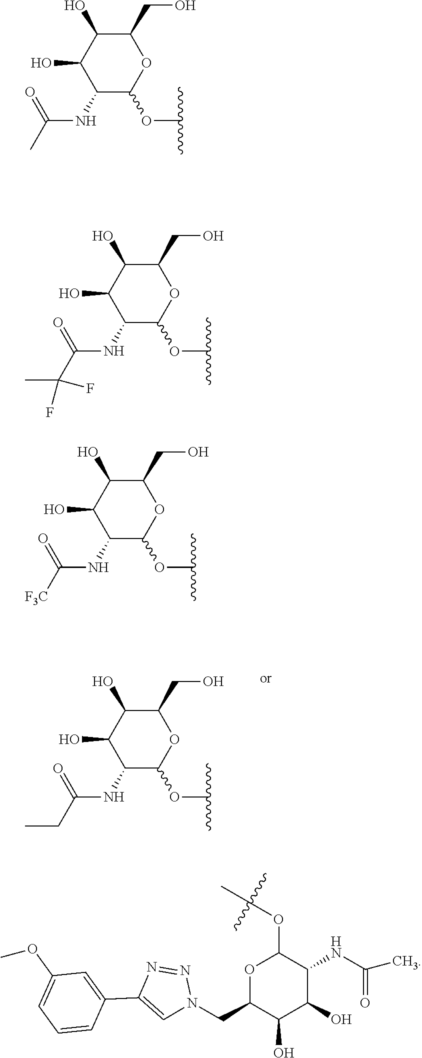 [SMILES: *OC1O[C@H](CO)[C@H](O)[C@H](O)[C@H]1NC(C)=O ; *OC1O[C@H](CO)[C@H](O)[C@H](O)[C@H]1NC(=O)C(C)(F)F ; *OC1O[C@H](CO)[C@H](O)[C@H](O)[C@H]1NC(=O)C(F)(F)F ; *OC1O[C@H](CO)[C@H](O)[C@H](O)[C@H]1NC(=O)CC ; *OC1O[C@H](Cn2cc(-c3cccc(OC)c3)nn2)[C@H](O)[C@H](O)[C@H]1NC(C)=O]

In one embodiment R² is:

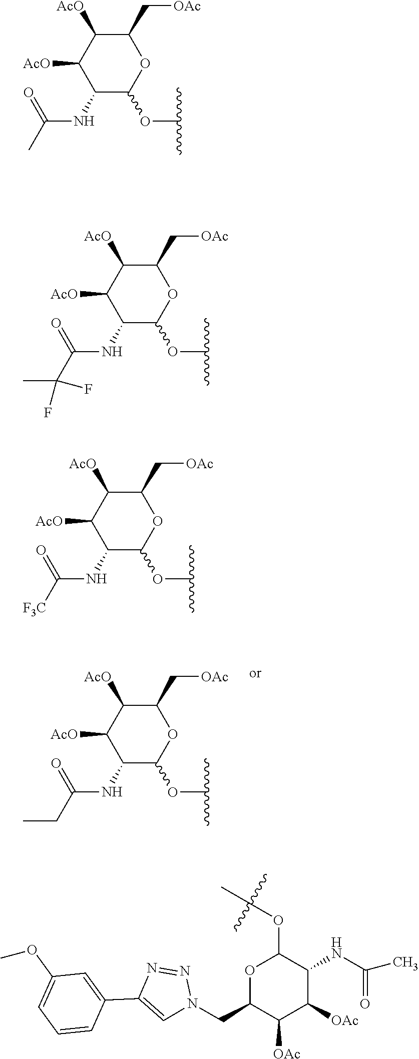 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1O[C@H](Cn2cc(-c3cccc(OC)c3)nn2)[C@H](OC(C)=O)[C@H](OC(C)=O)[C@H]1NC(C)=O]

In one embodiment L³ is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L³ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L³ is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In one embodiment L³ is:

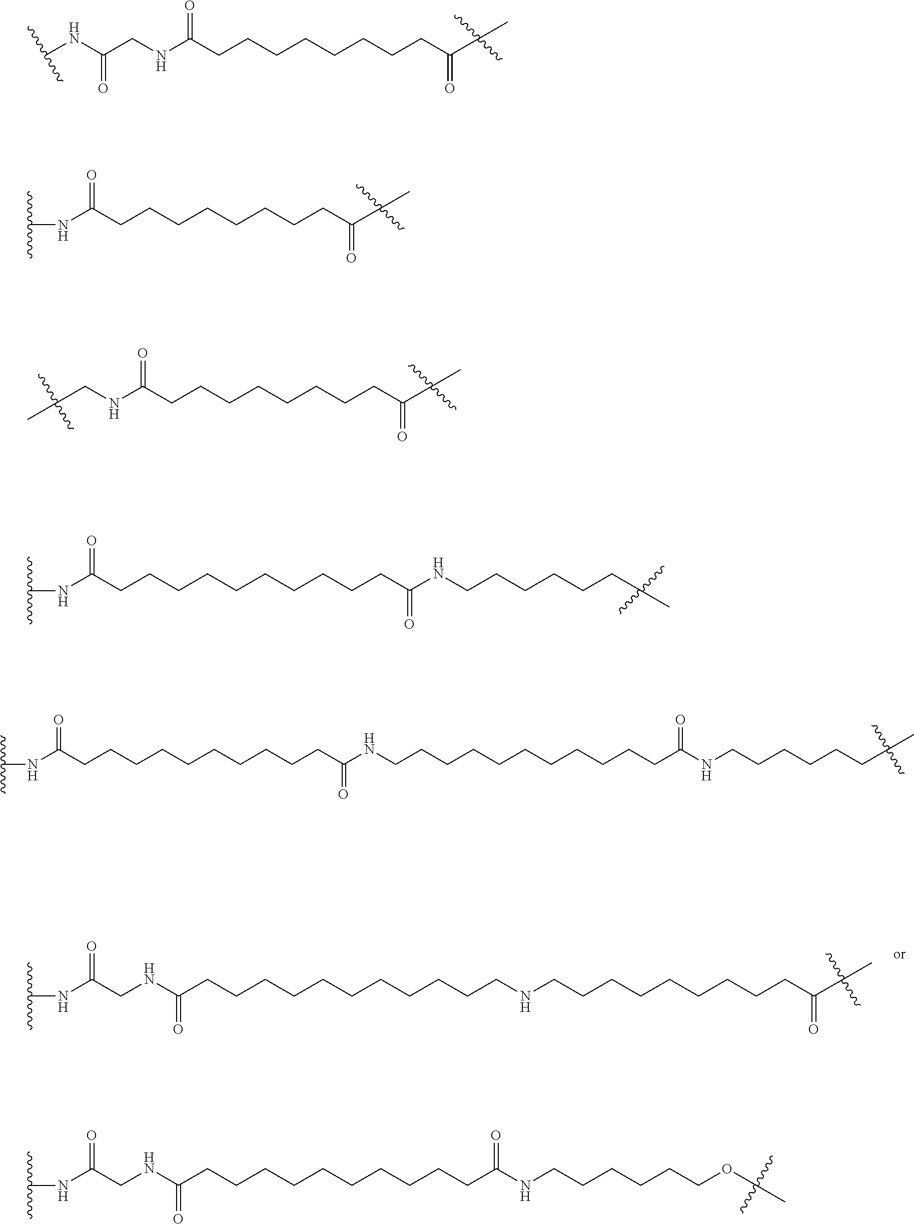

In one embodiment L³ is connected to B through —NH—, —O—, —S—, —(C═O)—, —(C═O)—NH—, —NH—(C═O)—, —(C═O)—O—, —NH—(C═O)—NH—, or —NH—(SO₂)—.

In one embodiment L⁴ is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L⁴ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L⁴ is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In one embodiment L⁴ is connected to R³ through —O—.

In one embodiment, the nucleic acid molecule R³ (e.g., siRNA) is attached to the reminder of the conjugate through the oxygen of a phosphate of the nucleic acid molecule.

In one embodiment, the nucleic acid molecule R³ (e.g., siRNA) is attached to the reminder of the conjugate through the oxygen of a phosphate at the 5′-end of the sense or the antisense strand.

In one embodiment, the nucleic acid molecule R³ (e.g., siRNA) is attached to the reminder of the conjugate through the oxygen of a phosphate at the 3′-end of the sense or the antisense strand.

In one embodiment, the nucleic acid molecule R³ (e.g., siRNA) is attached to the reminder of the conjugate through the oxygen of a phosphate at the 3′-end of the sense strand.

In one embodiment the group:

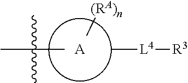

is selected from the group consisting of:

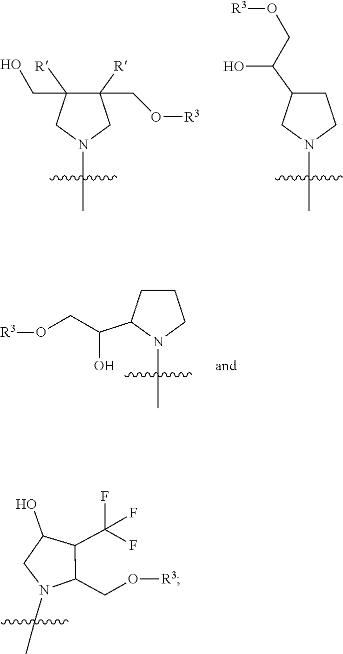

wherein

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In one embodiment the group:

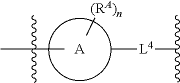

is selected from the group consisting of:

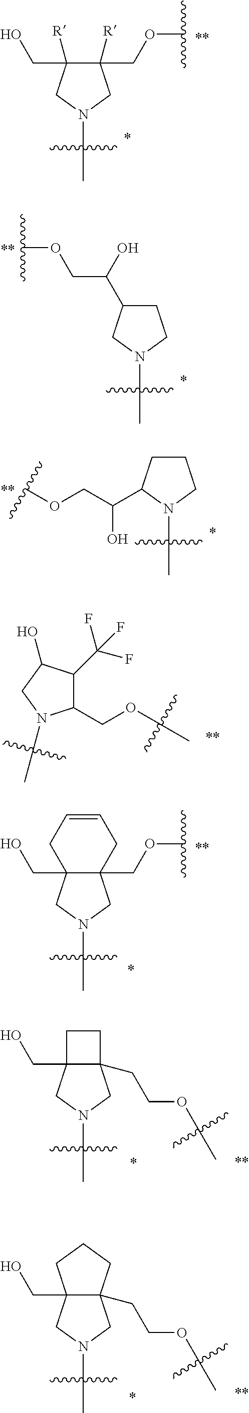

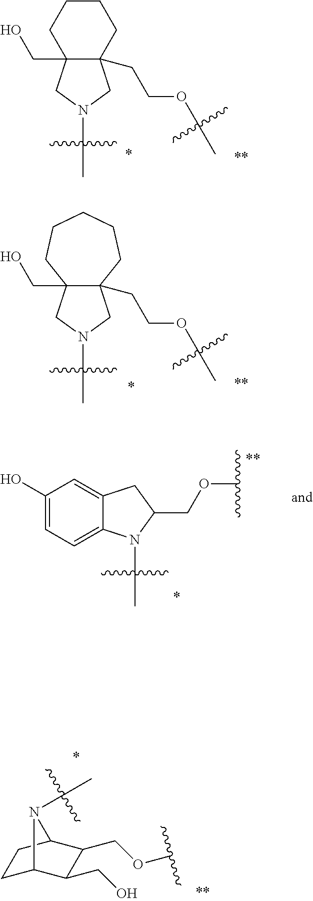

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L³; and

the valence marked with ** is attached to R³.

In one embodiment the group:

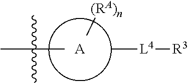

is:

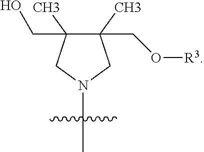

The invention also provides synthetic intermediates and methods disclosed herein that are useful to prepare conjugates of formula (I). For example, the invention includes a compound of formula (Ia):

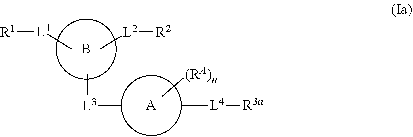

wherein:

R¹ is a saccharide;

L¹ is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo; B is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl

L² is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

R² is a saccharide;

L³ is absent or a linking group;

A is a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —OR^{a}, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

n is 0, 1, 2, 3, 4, 5, 6, 7, 8, 9, or 10;

L⁴ is absent or a linking group;

R^{3a} is H, a protecting group, a synthetic activating group, a covalent bond to a solid support, or a bond to a linking group that is bound to a solid support;

R^{a} is hydrogen, a protecting group, a covalent bond to a solid support, or a bond to a linking group L⁵ that is bound to a solid support; and

L⁵ is a linking group;

or a salt thereof.

In one embodiment R^{3a} is H.

In one embodiment R^{3a} is a protecting group. In one embodiment the protecting group is acetate, triflate, mesylate or succinate.

In one embodiment R^{3a} is a synthetic activating group. In one embodiment the synthetic activating group is derivable from DCC, HOBt, EDC, BOP, PyBOP or HBTU.

In one embodiment R^{3a} is a covalent bond to a solid support.

In one embodiment R^{3a} is a bond to a linking group that is bound to a solid support. In one embodiment the linking group that is bound to a solid support is —C(═O)CH₂CH₂C(═O)N(H)—.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

The following Schemes 1-22 illustrate the preparation of intermediate compounds that can be used to prepare conjugates of formula I. The intermediate compounds and the synthetic processes illustrated in Schemes 1-22 are embodiments of the present invention.

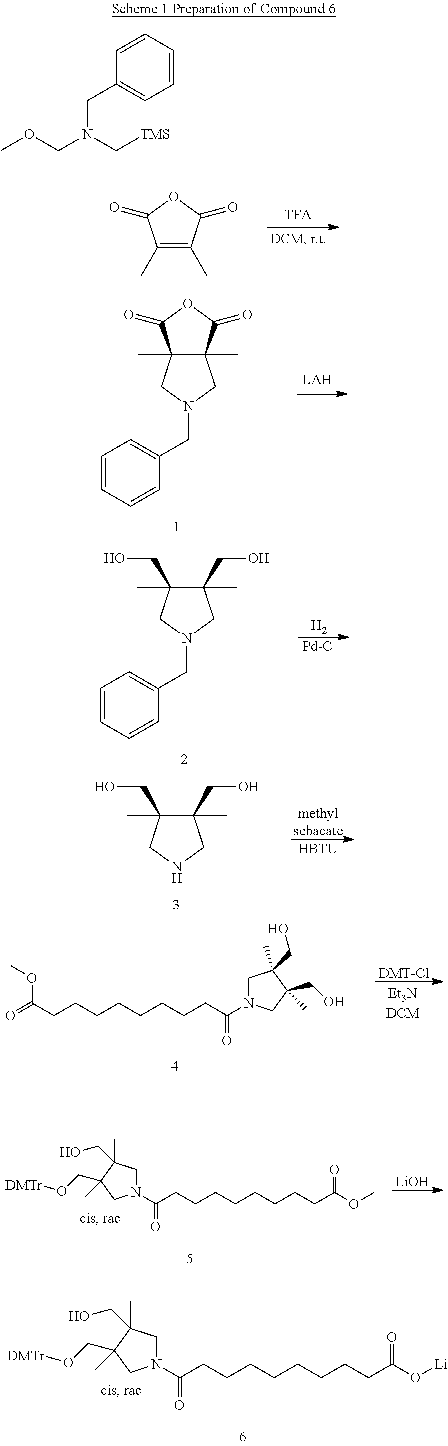

### Step 1. Preparation of (3aR,6aS)-5-Benzyl-3a,6a-dimethyltetrahydro-1H-furo[3,4-c]pyrrole-1,3(3aH)-dione 1

To a cooled solution (0° C.) of 3,4-dimethylfuran-2,5-dione (40 g, 317 mmol) and N-benzyl-1-methoxy-N-((trimethylsilyl)methyl)methanamine (94.1 g, 396.5 mmol) in DCM (600 ml) was slowly added trifluoroacetic acid (732 μl). Stir overnight allowing the solution to slowly warm to RT. The reaction mixture was concentrated to dryness, dissolved in EtOAc (500 ml), washed with saturated sodium bicarbonate (2×500 ml), dried on magnesium sulfate, filtered and concentrated to dryness. Purification by column chromatography on silica gel (gradient: 20% ethyl acetate in hexanes to 100% ethyl acetate) afforded (3aR,6aS)-5-Benzyl-3a,6a-dimethyltetrahydro-1H-furo[3,4-c]pyrrole-1,3(3aH)-dione as a yellow oil (53.7 g, 65%). Rf 0.85 40% EtOAc-Hexane

### Step 2. Preparation of ((3R,4S)-1-Benzyl-3,4-dimethylpyrrolidine-3,4-diyl)dimethanol 2

To a cooled (0° C.) solution of (3aR,6aS)-5-Benzyl-3a,6a-dimethyltetrahydro-1H-furo[3,4-c]pyrrole-1,3(3aH)-dione (53.7 g, 205.7 mmol) in anhydrous diethyl ether (750 ml) was added slowly lithium aluminum hydride pellets (17.6 g, 463 mmol) in portions over an afternoon. The solution was stirred overnight warming to room temperature as the ice water bath melted. Upon completion, the reaction was cooled to 0° C. and very slowly quenched with 25 ml of 5M NaOH followed by 12 ml of water. Stir for 30 minutes then add magnesium sulfate and filter. The filtrate was concentrated to afford ((3R,4S)-1-Benzyl-3,4-dimethylpyrrolidine-3,4-diyl)dimethanol as a colorless oil (33.6 g, 65%). Rf 0.25 10% CH₃OH—CH₂Cl₂

### Step 3. Preparation of ((3R,4S)-3,4-Dimethylpyrrolidine-3,4-diyl)dimethanol 3

To a solution of ((3R,4S)-1-Benzyl-3,4-dimethylpyrrolidine-3,4-diyl)dimethanol (40.1 g, 161 mmol) in methanol (300 ml) was added 10% palladium on activated charcoal wet (4 g). The solution was stirred vigorously under a hydrogen atmosphere for 16 hours. Upon completion the solution was filtered through Celite, and concentrated to dryness to afford ((3R,4S)-3,4-Dimethylpyrrolidine-3,4-diyl)dimethanol as a colorless solid (24 g, 94%). Rf 0.05 10% CH₃OH—CH₂Cl₂

### Step 4. Preparation of Methyl 10-((3R,4S)-3,4-bis(hydroxymethyl)-3,4-dimethylpyrrolidin-1-yl)-10-oxodecanoate 4

A solution of 3 (24 g, 151 mmol) and monomethyl sebacate (34.2 g, 159 mmol) in CH₂Cl₂ (1l) was treated with HBTU (62.9 g, 166 mmol) and Hunig's base (105 ml, 604 mmol). After stirring overnight the mixture was washed with NaHCO₃ (sat. aq.), water and brine, then dried (MgSO₄), filtered and concentrated. The crude material was subjected to chromatography (gradient: 0% CH₃OH—CH₂Cl₂ to 20%) to yield 4 (41.5 g, 77%). Rf 0.55 10% CH₃OH—CH₂Cl₂

### Step 5. Preparation of methyl 10-(3-((bis(4-methoxyphenyl)(phenyl)methoxy)methyl)-4-(hydroxymethyl)-3,4-dimethylpyrrolidin-1-yl)-10-oxodecanoate 5

A solution of 4 (41.5 g, 116 mmol) and 4,4′-Dimethoxytrityl chloride (38.8 g, 116 mmol) in pyridine (400 ml) was stirred overnight. The pyridine was then removed under reduced pressure and the crude material was subjected to chromatography (gradient: 0% CH₃OH—CH₂Cl₂ to 10%) to yield 5 (29.5 g, 39%) as a yellow oil. Rf 0.5 5% CH₃OH—CH₂Cl₂

### Step 6. Preparation of lithium 10-(3-((bis(4-methoxyphenyl)(phenyl)methoxy)methyl)-4-(hydroxymethyl)-3,4-dimethylpyrrolidin-1-yl)-10-oxodecanoate 6

To a solution of compound 5 (29.5 g, 45 mmol) in THF (250 ml) and water (250 ml) was added lithium hydroxide (1.19 g, 50 mmol). The solution was stirred for 18 hours at room temperature then concentrated to remove the THF. The remaining aqueous solution was freeze dried overnight to afford 6 as a pale purple solid (28.5 g, 98%). Rf 0.56 10% CH₃OH—CH₂Cl₂

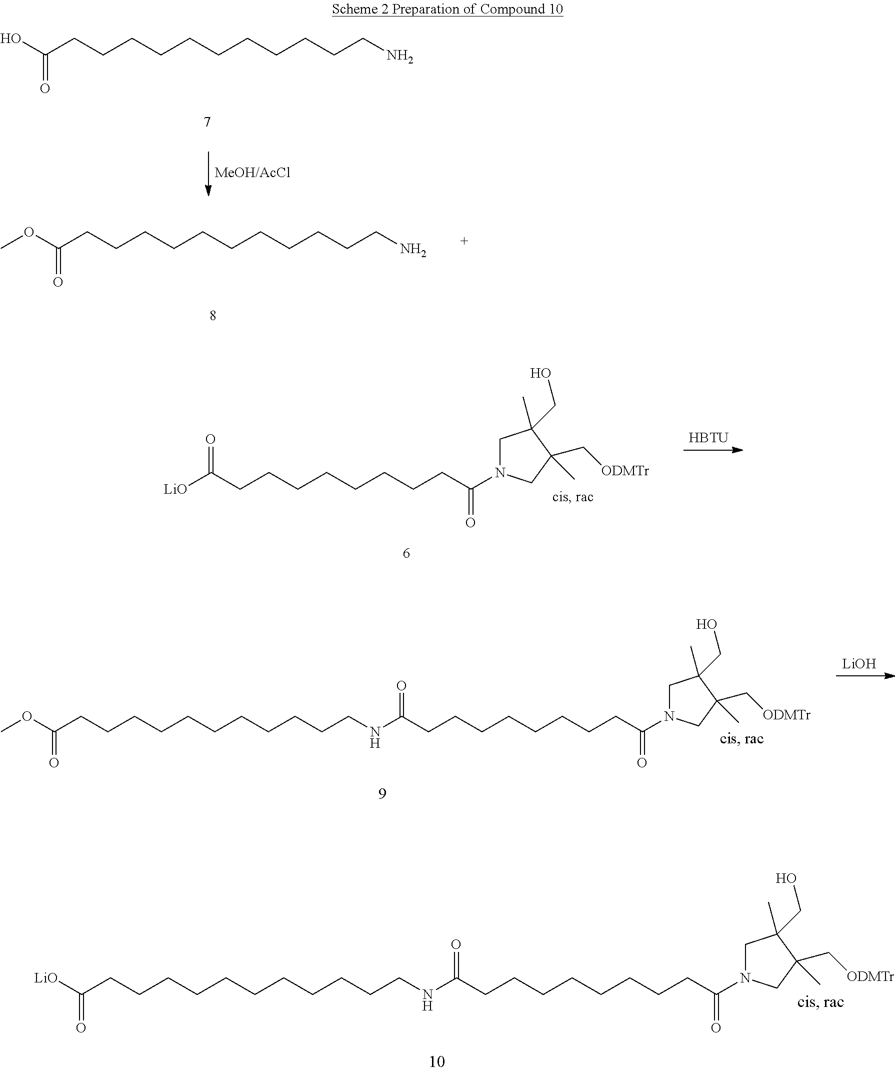

### Step. 1. Preparation of Methyl 12-Aminododecanoate 8

12-Aminoundecanoic acid 7 (10 g, 4.64 mmol) was stirred in MeOH at RT. Acetyl chloride (856 μl, 12 mmol) was added dropwise and the reaction stirred for 1.5 hr. The solvent was removed in-vacuo, the residue taken up in MTBE and chilled in the fridge overnight. The resultant precipitate was collected by filtration, washed with ice cold MTBE and dried under high vacuum to afford methyl 12-aminododecanoate 8.

### Step 2. Preparation of methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 9

Lithium 10-(3-((bis(4-methoxyphenyl)(phenyl)methoxy)methyl)-4-(hydroxymethyl)-3,4-dimethylpyrrolidin-1-yl)-10-oxodecanoate (6) (2 g, 3.1 mmol), methyl 12-aminododecanoate (8) (778 mg, 3.1 mmol), HBTU (1.2 g, 3.1 mmol) and TEA (1.4 ml, 10 mmol) were stirred in DCM at RT O/N. The precipitate was removed by filtration, the filtrate concentrated in-vacuo and the residue purified by column chromatography (5% MeOH, DCM). TLC showed two close running spots with identical mass that were assigned as geometric isomers and pooled together to methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate (9) in quantitative fashion.

### Step 3. Preparation of lithium 12-(10-(3-((bis(4-methoxyphenyl)(phenyl)methoxy)-methyl)-4-(hydroxymethyl)-3,4-dimethylpyrrolidin-1-yl)-10-oxodecanamido)-dodecanoate 10

Methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 9 (3.1 mmol) was stirred in THF:H₂O (50:50) with LiOH (88 mg, 3.7 mmol) at RT O/N. Reaction was confirmed by TLC and the THF removed in-vacuo. The aqueous solution was frozen in liquid N₂ and lyophilized for 48 hours to give lithium 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 10 quantitatively.

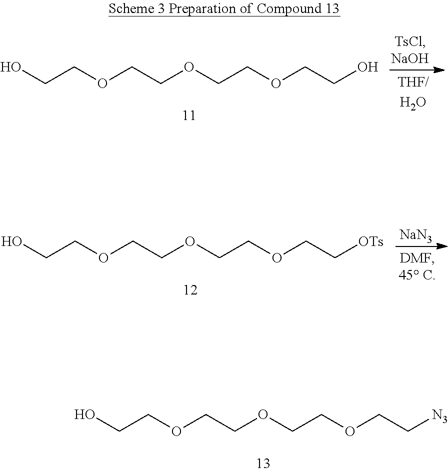 [SMILES: OCCOCCOCCOCCO ; Cc1ccc(S(=O)(=O)OCCOCCOCCOCCO)cc1 ; [N-]=[N+]=NCCOCCOCCOCCO ; "NaN3"]

### Step 1. Preparation of 2-(2-(2-(2-hydroxyethoxy)ethoxy)ethoxy)ethyl 4-methylbenzenesulfonate 12

A solution of tetraethylene glycol (11) (934 g, 4.8 mol) in THF (175 ml) and aqueous NaOH (5M, 145 ml) was cooled (0° C.) and treated with p-Toluensulfonyl chloride (91.4 g, 480 mmol) dissolved in THF (605 ml) and then stirred for two hours (0° C.). The reaction mixture was diluted with water (3 L) and extracted (3×500 ml) with CH₂Cl₂. The combined extracts were washed with water and brine then dried (MgSO₄), filtered and concentrated to afford 2-(2-(2-(2-hydroxyethoxy)ethoxy)ethoxy)ethyl 4-methylbenzenesulfonate (12) (140 g, 84%) as a pale yellow oil. Rf (0.57, 10% MeOH—CH₂Cl₂).

### Step 2. Preparation of 2-(2-(2-(2-azidoethoxy)ethoxy)ethoxy)ethan-1-ol 13

A solution of 12 (140 g, 403 mmol) in DMF (880 ml) was treated with sodium azide (131 g, 2.02 mol) and heated (45° C.) overnight. A majority of the DMF was removed under reduced pressure and the residue was dissolved in CH₂Cl₂ (500 ml) and washed (3×500 ml) with brine then dried (MgSO₄), filtered and concentrated. The residue was passed through a short bed of silica (5% MeOH—CH₂Cl₂) and concentrated to yield 2-(2-(2-(2-azidoethoxy)ethoxy)ethoxy)ethan-1-ol 13 (65 g, 74%) as a yellow oil. R_{f} (0.56, 10% MeOH—CH₂Cl₂).

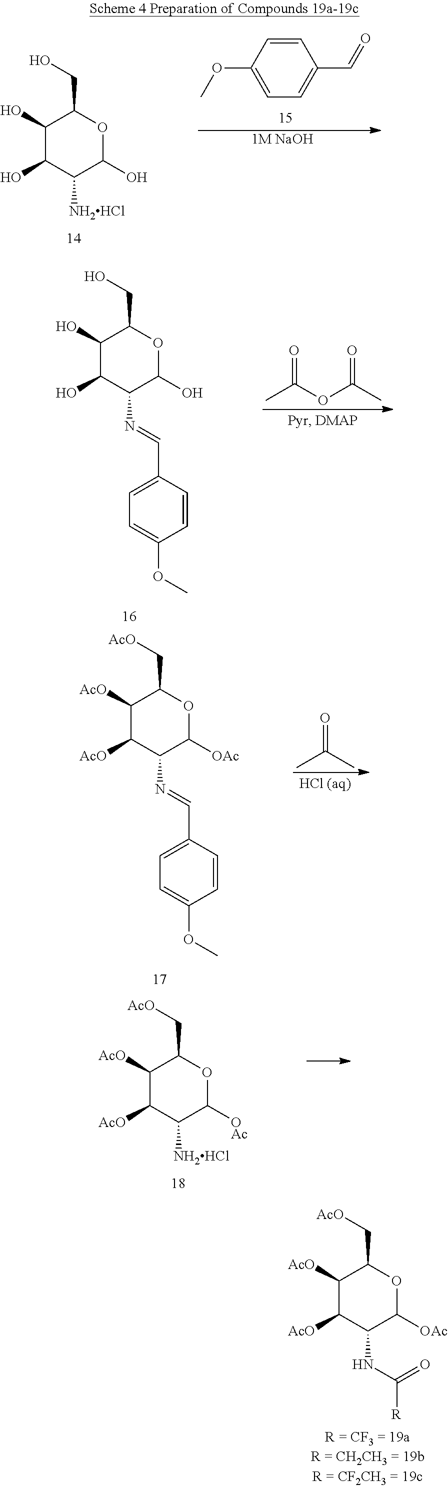

### Step 1. Preparation of (3R,4R,5R,6R)-6-(hydroxymethyl)-3-(((E)-4-methoxybenzylidene) amino)tetrahydro-2H-pyran-2,4,5-triol 16

D-Galactosamine HCl (14) (9 g, 41.7 mmol) was stirred in 1 M NaOH solution at RT. Anisaldehyde (51 ml, 420 mmol) was added and the reaction stirred vigorously until solidification. The solid reaction was kept at 4° C. for 16 h. Ice cold water (200 ml) was added and the resultant solid collected by filtration, washing with ice cold EtOH/Et₂O (1:1). The solid was dried to a constant weight to give (3R,4R,5R,6R)-6-(hydroxymethyl)-3-(((E)-4-methoxybenzylidene) amino)tetrahydro-2H-pyran-2,4,5-triol (16) (9.81 g, 78%).

### Step 2. Preparation of (3R,4R,5R,6R)-6-(acetoxymethyl)-3-(((E)-4-methoxybenzylidene)amino) tetrahydro-2H-pyran-2,4,5-triyl triacetate 17

(3R,4R,5R,6R)-6-(Hydroxymethyl)-3-(((E)-4-methoxybenzylidene)amino)tetrahydro-2H-pyran-2,4,5-triol (16) (9.81 g, 30 mmol) was stirred in pyridine at 0° C. Acetic anhydride (34 ml) followed by DMAP (100 mg, cat) was added and the reaction stirred for 16 h allowing to warm to RT slowly. The resultant solution was poured onto crushed ice and kept at 4° C. for 16 h. The reaction was extracted with EtOAc (×3) and the combined organics washed with H₂O and brine, dried (Na₂SO₄) and concentrated in-vacuo to give (3R,4R,5R,6R)-6-(acetoxymethyl)-3-(((E)-4-methoxybenzylidene)amino)tetrahydro-2H-pyran-2,4,5-triyl triacetate (17) (6.0 g, 43%).

### Step 3. Preparation of (3R,4R,5R,6R)-6-(acetoxymethyl)-3-aminotetrahydro-2H-pyran-2,4,5-triyl triacetate hydrochloride 18

(3R,4R,5R,6R)-6-(Acetoxymethyl)-3-(((E)-4-methoxybenzylidene)amino)tetrahydro-2H-pyran-2,4,5-triyl triacetate (17) (6.0 g, 43%) was heated at reflux in acetone (300 ml). HCl (aq) (5N, 3.0 ml) was added and the reaction stirred for 15 mins. After cooling, Et₂O (400 ml) was added and the reaction kept at 4° C. for 16 h. The resultant solid was collected by filtration, washing twice with ice cold Et₂O. The solid was dried to a constant weight to give (3R,4R,5R,6R)-6-(acetoxymethyl)-3-aminotetrahydro-2H-pyran-2,4,5-triyl triacetate hydrochloride (18) (4.17 g, 84.4%).

### Step 4a. Preparation of (3R,4R,5R,6R)-6-(acetoxymethyl)-3-(2,2,2-trifluoroacetamido) tetrahydro-2H-pyran-2,4,5-triyl triacetate 19a

(3R,4R,5R,6R)-6-(Acetoxymethyl)-3-aminotetrahydro-2H-pyran-2,4,5-triyl triacetate hydrochloride (18) (13.5 g, 35.2 mmol) and TEA (7.83 g, 77.4 mmol) were stirred in DCM at RT. TFAA (8.13 g, 38.7 mmol) in DCM was added dropwise and the reaction stirred for 1 h. The reaction was diluted with DCM, washed sequentially with 1M HCl, saturated NaHCO₃, water and brine, dried (Na₂SO₄) and concentrated in-vacuo. The residue was purified by automated flash chromatography (5% MeOH/DCM) to give (3R,4R,5R,6R)-6-(acetoxymethyl)-3-(2,2,2-trifluoroacetamido)tetrahydro-2H-pyran-2,4,5-triyl triacetate (19a) (9.64 g, 61.8%). Product confirmed by MS (ESI +ve).

### Step 4b. Preparation of (3R,4R,5R,6R)-6-(acetoxymethyl)-3-propionamidotetrahydro-2H-pyran-2,4,5-triyl triacetate 19b

This compound was prepared in an analogous fashion to (3R,4R,5R,6R)-6-(acetoxymethyl)-3-(2,2,2-trifluoroacetamido)tetrahydro-2H-pyran-2,4,5-triyl triacetate (19a) using propionic anhydride instead of TFAA to give (3R,4R,5R,6R)-6-(acetoxymethyl)-3-propionamidotetrahydro-2H-pyran-2,4,5-triyl triacetate (19b) (1.2 g, 85.3%). Product confirmed by MS (ESI +ve).

### Step 4c. Preparation of (3R,4R,5R,6R)-6-(acetoxymethyl)-3-(2,2-difluoropropanamido) tetrahydro-2H-pyran-2,4,5-triyl triacetate 19c

(3R,4R,5R,6R)-6-(Acetoxymethyl)-3-aminotetrahydro-2H-pyran-2,4,5-triyl triacetate hydrochloride (18) (15.34 g, 39.98 mmol), 2,2-difluoropropionic acid (4.4 g, 39.98 mmol), HATU (24.37 g, 64 mmol) and TEA (12.14 g, 120 mmol) were stirred in DMF at RT for 16 h. The reaction was partitioned between EtOAc and water. The organics were separated, washed sequentially with 1M HCl, saturated NaHCO₃, water and brine, dried (Na₂SO₄) and concentrated in-vacuo. The residue was purified by automated flash chromatography (3% MeOH/DCM) to give (3R,4R,5R,6R)-6-(acetoxymethyl)-3-(2,2-difluoropropanamido)tetrahydro-2H-pyran-2,4,5-triyl triacetate (19c) (15.8 g, 90%). Product confirmed by MS (ESI +ve).

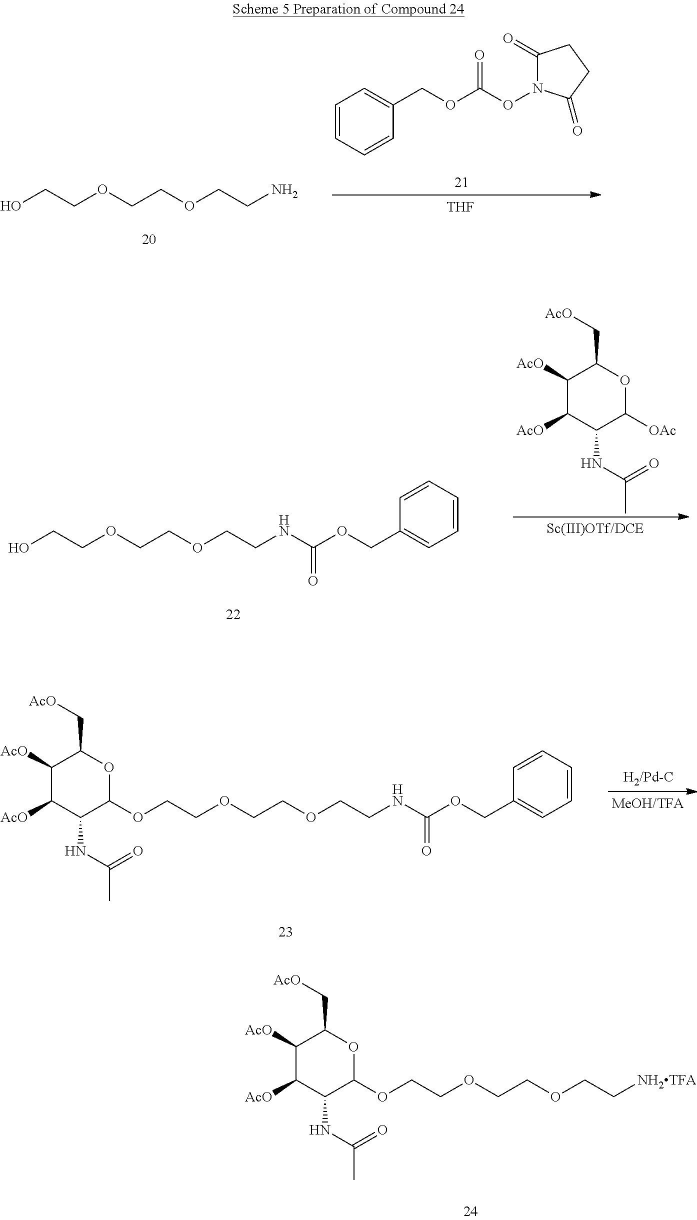

### Step 1. Preparation of benzyl (2-(2-(2-hydroxyethoxy)ethoxy)ethyl)carbamate 22

A solution of the amino alcohol (20) (313.6 g, 2.1 mol) in THF (3.5 L) was treated, portion-wise, with N-(Benzyloxycarbonyloxy)succinimide (21) (550 g, 2.21 mol). Once the reaction was complete (18 h) the THF was removed under reduced pressure and the residue dissolved in CH₂Cl₂ (2.5 L), then washed with an equal volume of HCl (1 M), NaHCO₃ (Sat. Aq.), H₂O and brine. The organic extract was dried (MgSO₄), filtered and concentrated. The crude material (600 g) was subjected to chromatography (4 kg silica; 1-12% CH₃OH—CH₂Cl₂) to yield HO-Trig-NHZ (22) (468 g, 78%) as a clear-yellow viscous oil.

### Step 2. Preparation of (2R,3R,4R,5R)-5-acetamido-2-(acetoxymethyl)-6-((3-oxo-1-phenyl-2,7,10-trioxa-4-azadodecan-12-yl)oxy)tetrahydro-2H-pyran-3,4-diyl diacetate 23

A heterogeneous mixture of galactosamine pentaacetate (715.2 g, 1.84 mol) and HO-Trig-NHZ (22) (400 g, 1.41 mol) in 1,2 dichloroethane (10 L) was treated with 5 mol % Sc(OTf)₃ (34.6 g, 70.5 mmol) and heated (85° C.). After stirring (5.5 h) the solution became clear and homogeneous, the reaction was cooled and washed with NaHCO₃ (Sat. Aq.), HCl (1M), H₂O and brine. The organic extracts were dried (MgSO₄), filtered and concentrated. The crude material (900 g) was treated with EtOAc (900 ml) which gave a milky heterogeneous mixture that was filtered through a course frit thus removing residual pentaacetate. The filtrate was concentrated, and the crude material was subjected to chromatography (5 kg silica; 0-10% CH₃OH-EtOAc) to yield the glycosylation product (23) (751 g, 87%) as a light brown foam.

### Step 3. Preparation of (2R,3R,4R,5R)-5-acetamido-2-(acetoxymethyl)-6-(2-(2-(2-((2,2,2-trifluoroacetyl)-14-azaneyl)ethoxy)ethoxy)ethoxy)tetrahydro-2H-pyran-3,4-diyl diacetate 24

A solution of Gal-trig-NHZ (23) (750 g, 1.22 mol), TFA (103.8 ml, 1.35 mol) and Pd/C (10%—wet support, 75 g) was purged with H₂. After vigorous stirring (4.5 h) the reaction mixture was purged with N₂ (30 min) then filtered through Celite and concentrated. The resultant brown foam (712 g, 99%) was used in the next step without further processing.

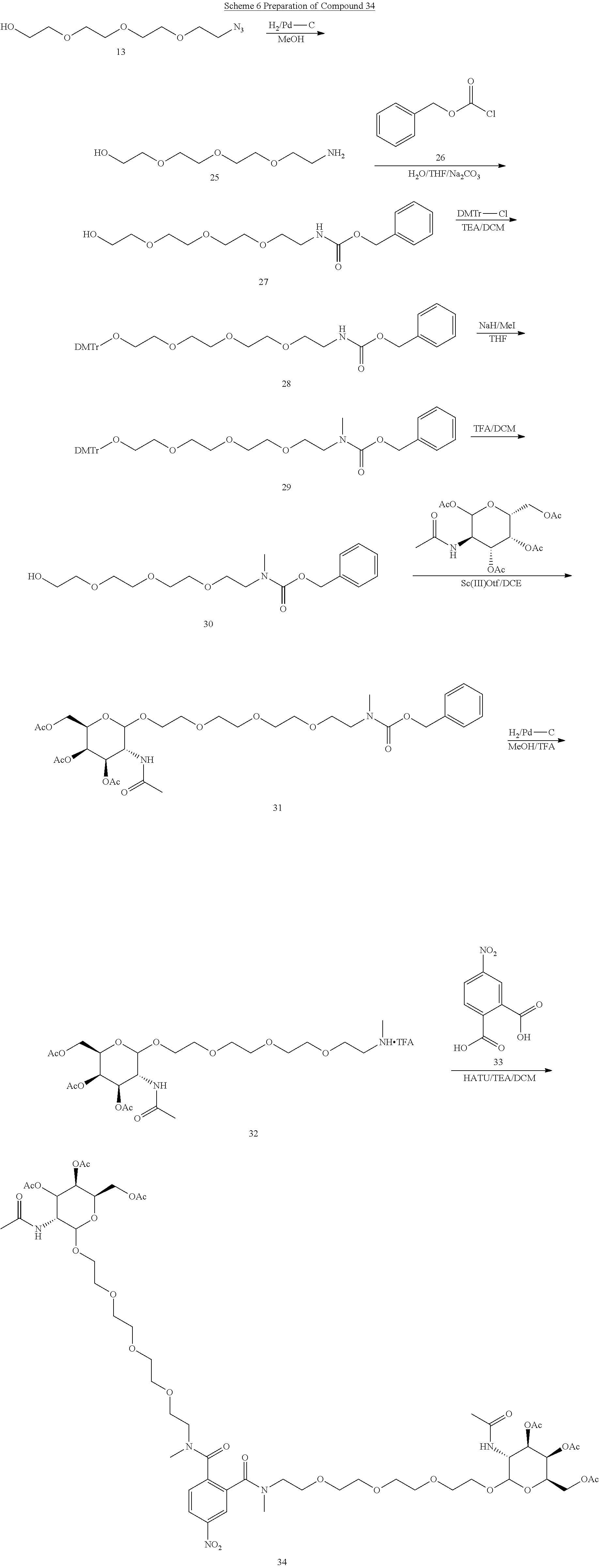

### Step 1. Preparation of 2-(2-(2-(2-aminoethoxy)ethoxy)ethoxy)ethan-1-ol 25

2-(2-(2-(2-Azidoethoxy)ethoxy)ethoxy)ethan-1-ol (13) (70.0 g, 318 mmol) was stirred in MeOH at RT. The reaction was hydrogenated over 10% PD-C (7 g) for 16 h. The reaction was filtered through celite and concentrated in-vacuo to give 2-(2-(2-(2-aminoethoxy)ethoxy)ethoxy)ethan-1-ol (25) (61.4 g, 100%) which was used without further purification. Product confirmed by MS (ESI +ve).

### Step 2. Preparation of benzyl (2-(2-(2-(2-hydroxyethoxy)ethoxy)ethoxy)ethyl)-carbamate 27

2-(2-(2-(2-Aminoethoxy)ethoxy)ethoxy)ethan-1-ol (25) (61.4 g, 318 mmol) was stirred in H₂O (500 ml) with Na₂CO₃ (50.51 g, 476 mmol) at 5° C. Benzyl chloroformate (26) (65.0 g, 381 mmol) in THF (480 ml) was added dropwise and the reaction stirred for 16 h allowing to warm to RT. THF was removed in-vacuo and the aqueous layer extracted with EtOAc (×3). The combined organics were dried (Na₂SO₄), concentrated in-vacuo and the residue purified by automated flash chromatography (5% MeOH/DCM) to give benzyl (2-(2-(2-(2-hydroxyethoxy)ethoxy)ethoxy) ethyl)carbamate (27) (23.6 g, 22.7%). Product confirmed by MS (ESI +ve).

### Step 3. Preparation of benzyl (1,1-bis(4-methoxyphenyl)-1-phenyl-2,5,8,11-tetraoxatridecan-13-yl)carbamate 28

(2-(2-(2-(2-Hydroxyethoxy)ethoxy)ethoxy)ethyl)carbamate (27) (23.6 g, 72.1 mmol) and TEA (7.7 g, 75.7 mmol) were stirred in DCM at RT. DMTr-Cl (25.65 g, 75.7 mmol) was added and the reaction stirred at RT for 2 h. The reaction was washed sequentially with saturated NaHCO₃, water and brine, dried (Na₂SO₄) and concentrated in-vacuo. The residue was purified by automated flash chromatography (50% EtOAc/Hex) to give (1,1-bis(4-methoxyphenyl)-1-phenyl-2,5,8,11-tetraoxatridecan-13-yl)carbamate (28) (25.5 g, 56.2%). Product confirmed by MS (ESI +ve).

### Step 4. Preparation of benzyl (1,1-bis(4-methoxyphenyl)-1-phenyl-2,5,8,11-tetraoxatridecan-13-yl)(methyl)carbamate 29

(1,1-Bis(4-methoxyphenyl)-1-phenyl-2,5,8,11-tetraoxatridecan-13-yl)carbamate (28) (25.5 g, 40.5 mmol) and Mel (46.0 g, 324 mmol) were stirred in dry THF at 0° C. NaH (60% dispersion in mineral oil) (2.92 g, 121.5 mmol) was added and the reaction stirred at 0° C. then at RT for 1 h. The reaction was partitioned between EtOAc and H₂O. The organics were separated, dried (Na₂SO₄) and concentrated in-vacuo. The residue was purified by automated flash chromatography (50% EtOAc/Hex) to give benzyl (1,1-bis(4-methoxyphenyl)-1-phenyl-2,5,8,11-tetraoxatridecan-13-yl)(methyl)carbamate (29) (26.06 g, 100%). Product confirmed by MS (ESI +ve).

### Step 5. Preparation of benzyl (2-(2-(2-(2-hydroxyethoxy)ethoxy)ethoxy)ethyl)(methyl) carbamate 30

Benzyl (1,1-bis(4-methoxyphenyl)-1-phenyl-2,5,8,11-tetraoxatridecan-13-yl)(methyl)carbamate (29) (26.06 g, 40.5 mmol) was stirred in DCM at RT. TFA (5.1 g, 44.5 mmol) was added and stirred for 1 h. 2 additional equivalents of TFA were added and the reaction stirred for 16 h. The reaction was concentrated in-vacuo and the residue purified by automated flash chromatography (5% MeOH/DCM) to give benzyl (2-(2-(2-(2-hydroxyethoxy)ethoxy)ethoxy)ethyl)(methyl) carbamate (30) (6.76 g, 48.9%). Product confirmed by MS (ESI +ve).

### Step 6. Preparation of (2R,3R,4R,5R)-5-acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate 31

(2-(2-(2-(2-Hydroxyethoxy)ethoxy)ethoxy)ethyl)(methyl) carbamate (30) (6.76 g, 19.8 mmol), (3R,4R,5R,6R)-3-acetamido-6-(acetoxymethyl)tetrahydro-2H-pyran-2,4,5-triyl triacetate (7.71 g, 19.8 mmol) and Sc(III)OTf (0.49 g, 1.0 mmol) were heated at reflux in DCE for 2 h. After cooling, the reaction was quenched with TEA and washed sequentially with 1M HCl, saturated NaHCO₃, water and brine, dried (Na₂SO₄) and concentrated in-vacuo. The residue was purified by automated flash chromatography to give (2R,3R,4R,5R)-5-acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate (31) (9.37 g, 70.6%). Product confirmed by MS (ESI +ve).

### Step 7. Preparation of (2R,3R,4R,5R)-5-acetamido-2-(acetoxymethyl)-6-((1,1,1-trifluoro-3-methyl-2-oxo-6,9,12-trioxa-3l4-azatetradecan-14-yl)oxy)tetrahydro-2H-pyran-3,4-diyl diacetate 32

(2R,3R,4R,5R)-5-Acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate (31) (9.37 g, 14.0 mmol) and TFA (1.76 g, 15.4 mmol) were stirred in MeOH at RT. The reaction was hydrogenated over 10% Pd—C (1 g) for approx. 2 h. The reaction was filtered through celite and concentrated in-vacuo to give (2R,3R,4R,5R)-5-acetamido-2-(acetoxymethyl)-6-((1,1,1-trifluoro-3-methyl-2-oxo-6,9,12-trioxa-3l4-azatetradecan-14-yl)oxy)tetrahydro-2H-pyran-3,4-diyl diacetate (32) (9.0 g, 98.9%). The product was used without purification. Product confirmed by MS (ESI +ve).

### Step 8. Preparation of (2R,2′R,3R,3′R,4R,4′R,5R,5′R)-(((4-nitro-1,2-phenylene)bis(2-methyl-1-oxo-5′,8′,11′-trioxa-2′-azatridecane-1,13-diyl))bis(oxy))bis(5-acetamido-2-(acetoxymethyl)tetrahydro-2H-pyran-6,3,4-triyl) tetraacetate 34

(2R,3R,4R,5R)-5-Acetamido-2-(acetoxymethyl)-6-((1,1,1-trifluoro-3-methyl-2-oxo-6,9,12-trioxa-3l4-azatetradecan-14-yl)oxy)tetrahydro-2H-pyran-3,4-diyl diacetate (32) (4.5 g, 6.93 mmol), 4-nitrophthalic acid (33) (0.73 g, 3.46 mmol), HATU (8.45 g, 22.18 mmol) and TEA (4.21 g, 41.6 mmol) were stirred in DCM at RT for 16 h. The reaction was diluted with DCM and washed sequentially with 1M HCl, saturated NaHCO₃, water and brine, dried (Na₂SO₄) and concentrated in-vacuo. The residue was purified by automated flash column chromatography (10% MeOH/DCM) to give (2R,2′R,3R,3′R,4R,4′R,5R,5′R)-(((4-nitro-1,2-phenylene)bis(2-methyl-1-oxo-5′,8′,11′-trioxa-2′-azatridecane-1,13-diyl))bis(oxy))bis(5-acetamido-2-(acetoxymethyl)tetrahydro-2H-pyran-6,3,4-triyl) tetraacetate (34) (5.0 g, 57.4%). Product confirmed by MS (ESI +ve).

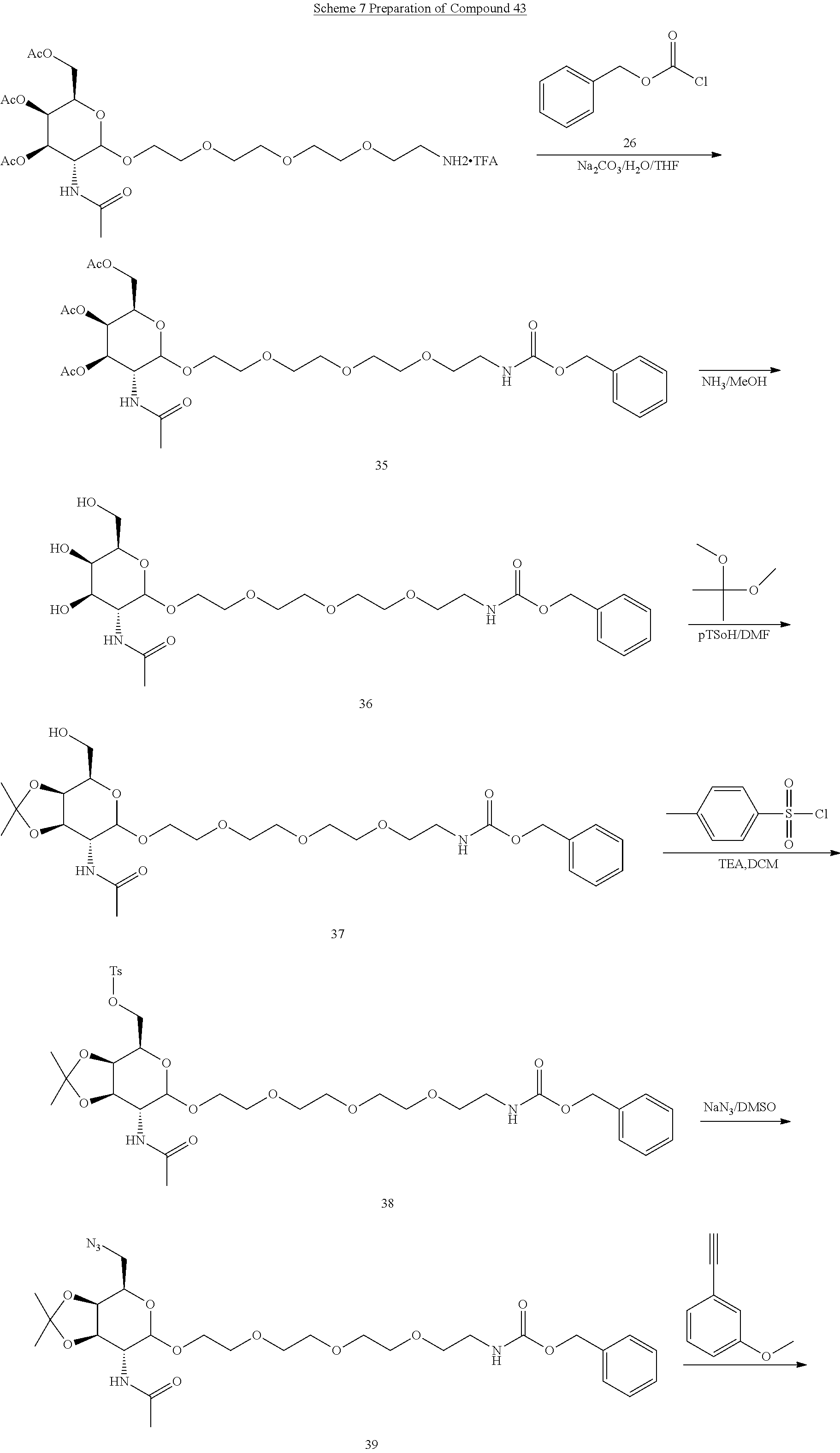

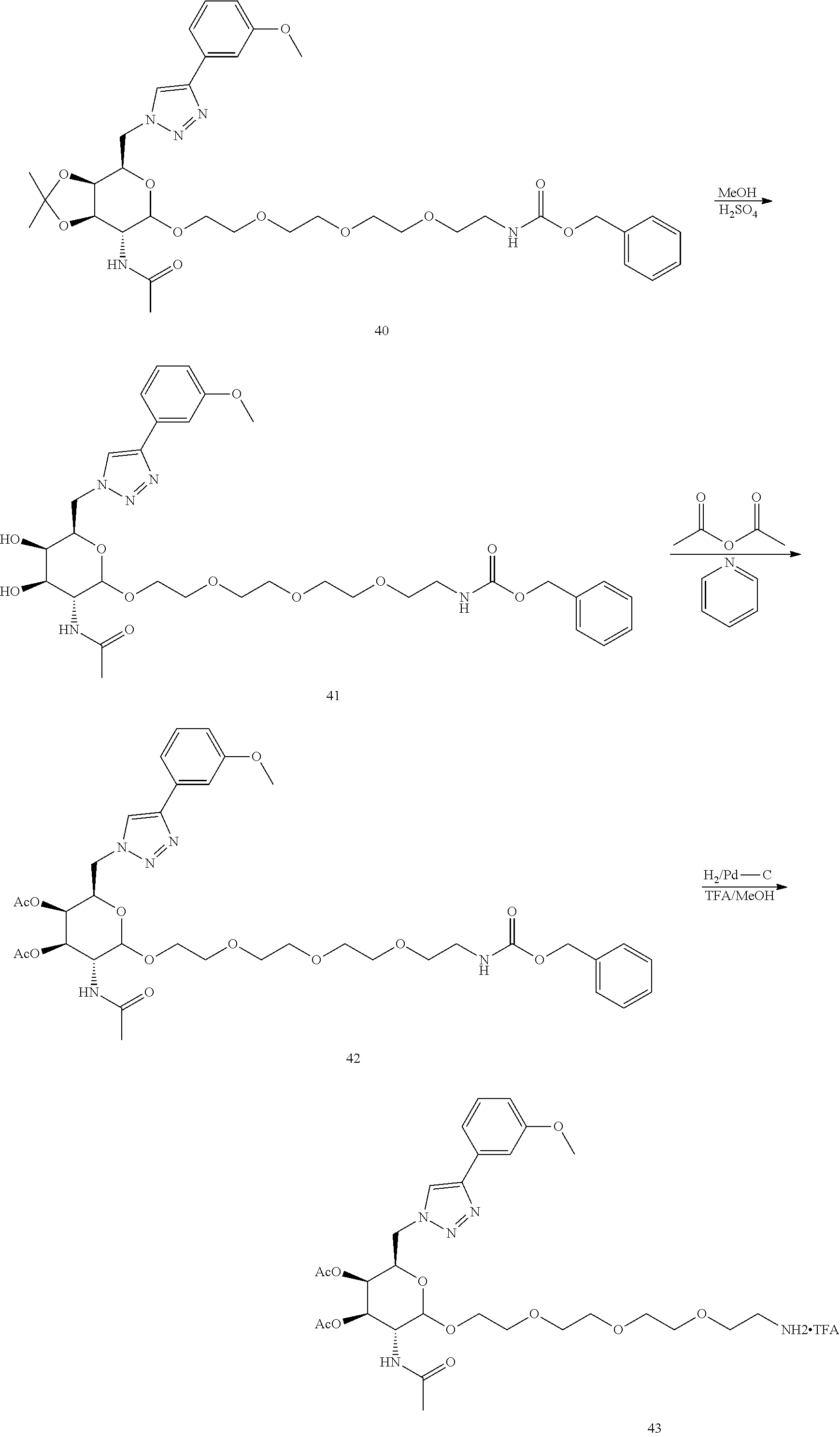

### Step 1. Preparation of (2R,3R,4R,5R)-5-acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate

(2R,3R,4R,5R)-5-Acetamido-2-(acetoxymethyl)-6-((1,1,1-trifluoro-2-oxo-6,9,12-trioxa-3l4-azatetradecan-14-yl)oxy)tetrahydro-2H-pyran-3,4-diyl diacetate (45.0 g, 70.8 mmol) and Na₂CO₃ (11.3 g, 106 mmol) were stirred in THF/H₂O (50:50) at RT. Benzyl chloroformate (26) (14.5 g, 85 mmol) was added dropwise and the reaction stirred for 16 h. THF was removed in-vacuo and the aqueous extracted with EtOAc (×3). The organics were washed sequentially with 1M HCl, saturated NaHCO₃, water and brine, dried (Na₂SO₄) and concentrated in-vacuo. The residue was purified by automated flash chromatography (5% MeOH/DCM) to give (2R,3R,4R,5R)-5-acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate (35) (25.12 g, 54%). Product confirmed by MS (ESI +ve).

### Step 2. Preparation of benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate 36

(2R,3R,4R,5R)-5-Acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate (35) (25.12 g, 38.3 mmol) was stirred in 7N ammonia solution in MeOH in an airtight sealed reaction vessel at RT for 16 h. The reaction was allowed to evaporate at 50° C. to remove ammonia and the remainder concentrated in-vacuo to give benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate (36) (20.3 g, 100%) which was used in subsequent reactions without further purification. Product confirmed by MS (ESI +ve).

### Step 3. Preparation of benzyl (2-(2-(2-(2-(((3aR,4R,7R,7aR)-7-acetamido-4-(hydroxymethyl)-2,2-dimethyltetrahydro-4H-[1,3]dioxolo[4,5-c]pyran-6-yl)oxy)-ethoxy)ethoxy)ethoxy)ethyl) carbamate 37

Benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate (36) (20.3 g, 38.3 mmol) was stirred in DMF (200 ml) at RT. 2,2-Dimethoxy propane (274 g, 1.6 mol) and pTsOH (cat) were added and the reaction heated at 65° C. for 16 h. The reaction was cooled to RT, TEA (20 ml) added and stirred for 30 min. The solvent was removed in-vacuo, the residue taken up in MeOH/H₂O (10:1) and the reaction refluxed for 1 h. The reaction was concentrated in-vacuo (azeotroping with toluene (×2) and the residue purified by automated flash chromatography (10% MeOH/DCM) to give benzyl (2-(2-(2-(2-(((3aR,4R,7R,7aR)-7-acetamido-4-(hydroxyl-methyl)-2,2-dimethyltetrahydro-4H-[1,3]dioxolo[4,5-c]pyran-6-yl)oxy)ethoxy)ethoxy)-ethoxy)ethyl)carbamate (37) (24.9 g, 100%). Product confirmed by MS (ESI +ve).

### Step 4. Preparation of ((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate 38

Benzyl (2-(2-(2-(2-(((3aR,4R,7R,7aR)-7-acetamido-4-(hydroxymethyl)-2,2-dimethyl-tetrahydro-4H-[1,3]dioxolo[4,5-c]pyran-6-yl)oxy)ethoxy)ethoxy)ethoxy)ethyl)carbamate (37) (25.5 g, 44.8 mmol) and TEA (9.97 g, 98.5 mmol) were stirred in DCM at 0° C. p-Toluene-sulfonyl chloride (18.8 g, 98.5 mmol) in DCM was added and the reaction stirred for 16 h allowing to warm to RT. The reaction was diluted with DCM, washed sequentially with 1M HCl, saturated NaHCO₃, water and brine, dried (Na₂SO₄) and concentrated in-vacuo. The residue was purified by automated flash chromatography (5% MeOH/DCM) to give ((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate (38) (25.5 g, 78.8%). Product confirmed by MS (ESI +ve).

### Step 5. Preparation of benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)-ethyl) carbamate 39

((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate (38) (25.0 g, 34.5 mmol) and NaN₃ (28.7 g, 434.6 mmol) were heated in DMSO/H₂O (200 ml/20 ml) at 100° C. for 12 h. The reaction was cooled and partitioned between EtOAc and saturated NaHCO₃. The aqueous was further extracted another two times and the combined organics washed with saturated NaHCO₃, water and brine, dried (Na₂SO₄) and concentrated in-vacuo. The residue was purified by automated flash chromatography (5% MeOH/DCM) to give benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)ethyl) carbamate (39) (16.1 g, 78.2%). Product confirmed by MS (ESI +ve).

### Step 6. Preparation of benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy)ethoxy)ethyl)carbamate 40

Benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)ethyl)carbamate (39) (16.1 g, 27.0 mmol) was stirred in MeOH (200 ml) at RT. 1-Ethynyl-3-methoxybenzene (4.28 g, 32.4 mmol), tris(benzyltriazolylmethyl)amine (0.72 g, 1.35 mmol), CuSO₄ (0.07 g, 0.27 mmol in 1 ml H₂O) and sodium ascorbate (0.53 g, 2.7 mmol in 5 ml H₂O) were added sequentially and the reaction stirred at RT for 16 h. The solvent was removed in-vacuo, the residue taken up in DCM (200 ml) and washed with water. The aqueous layer was back extracted with DCM and the combined organics washed with brine and dried (Na₂SO₄). The reaction was concentrated in-vacuo and the residue purified by automated flash chromatography (10% MeOH/EtOAc) to give benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy)ethoxy)ethyl) carbamate (40) (15.0 g, 76.4%). Product confirmed by MS (ESI +ve).

### Step 7. Preparation of benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy) ethoxy)ethoxy)ethyl)carbamate 41

Benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy) ethoxy)ethyl)carbamate (40) (15.0 g, 20.6 mmol) was stirred in MeCN (200 ml) and 1.84% H₂SO₄ (180 ml) at RT for 96 h. The reaction was extracted with EtOAc (3×250 ml), washed with saturated NaHCO₃, water and brine, dried (Na₂SO₄) and concentrated in-vacuo to give benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy)ethoxy)ethoxy)ethyl)-carbamate (41) (11.0 g, 16.0 mmol). the product was used in crude in subsequent reactions. Product confirmed by MS (ESI +ve).

### Step 8. Preparation of (2R,3S,4R,5R)-5-acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate 42

Benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy)ethoxy)ethoxy)-ethyl)carbamate (41) (11.0 g, 16.0 mmol) was stirred in pyridine (200 ml) at RT. Acetic anhydride (16.3 g, 160 mmol) was added and the reaction stirred for 16 h at RT followed by 50° C. for 3 h. The reaction was poured over water and extracted three times with DCM (250 ml). The combined organics were washed with saturated NaHCO₃ (×2), 1N HCl (×2), water and brine, dried (Na₂SO₄) and concentrated in-vacuo. The residue was purified by automated flash chromatography (5% MeOH/DCM) to give (2R,3S,4R,5R)-5-acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate (42) (10.7 g, 86.7%). Product confirmed by MS (ESI +ve).

### Step 9. Preparation of (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-3l4-azatetradecan-14-yl)oxy)tetrahydro-2H-pyran-3,4-diyl diacetate 43

(2R,3 S,4R,5R)-5-Acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate (42) (9.06 g, 11.74 mmol) and TFA (1.47 g, 12.91 mmol) were stirred in MeOH at RT. The reaction was hydrogenated over 10% Pd—C for 1 h. The reaction was filtered through celite and concentrated in-vacuo to give (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-3l4-azatetradecan-14-yl)oxy)tetrahydro-2H-pyran-3,4-diyl diacetate (43) (8.8 g, 99.7%) which was used in subsequent reactions without purification. Product confirmed by MS (ESI +ve).

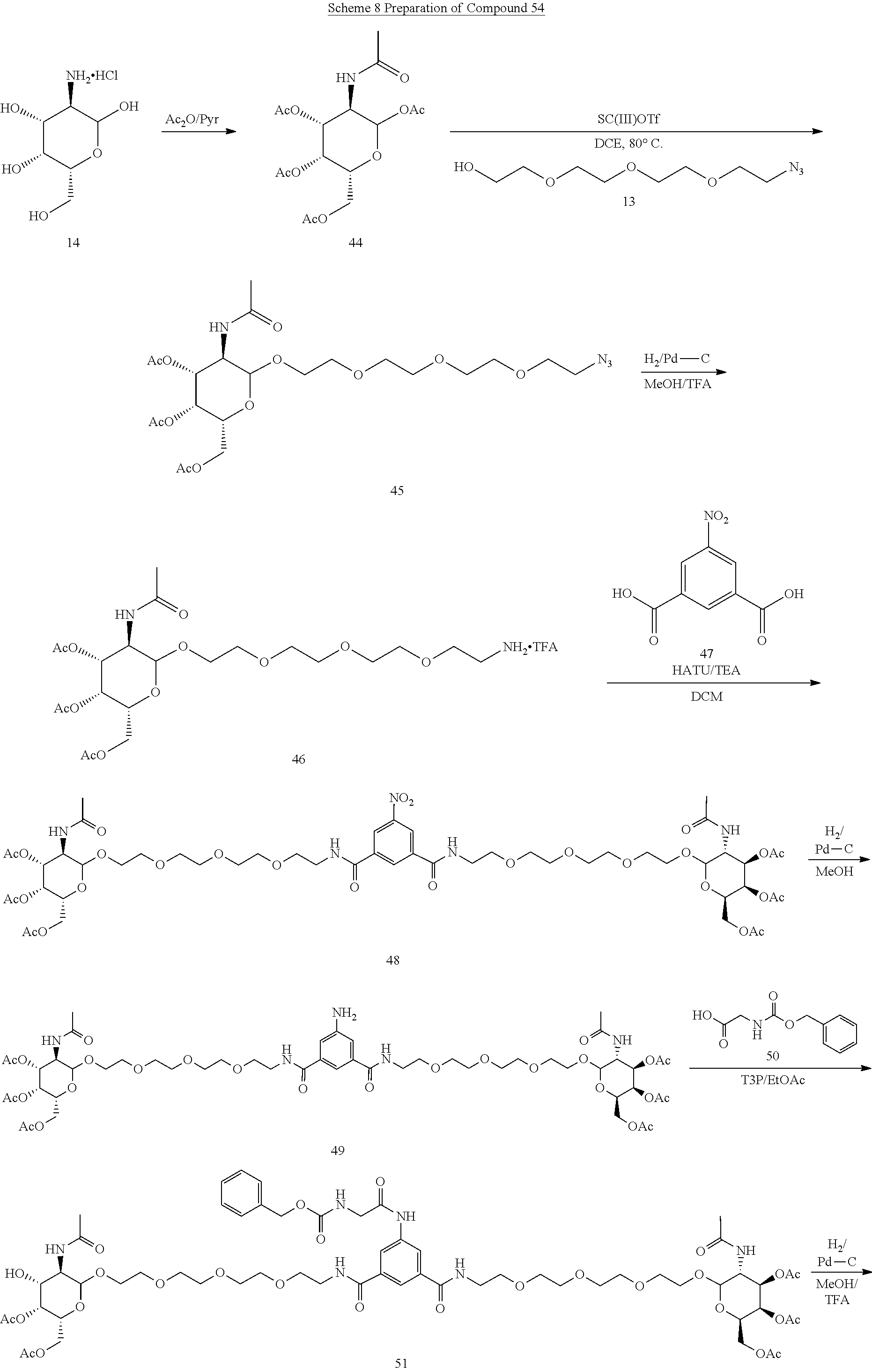

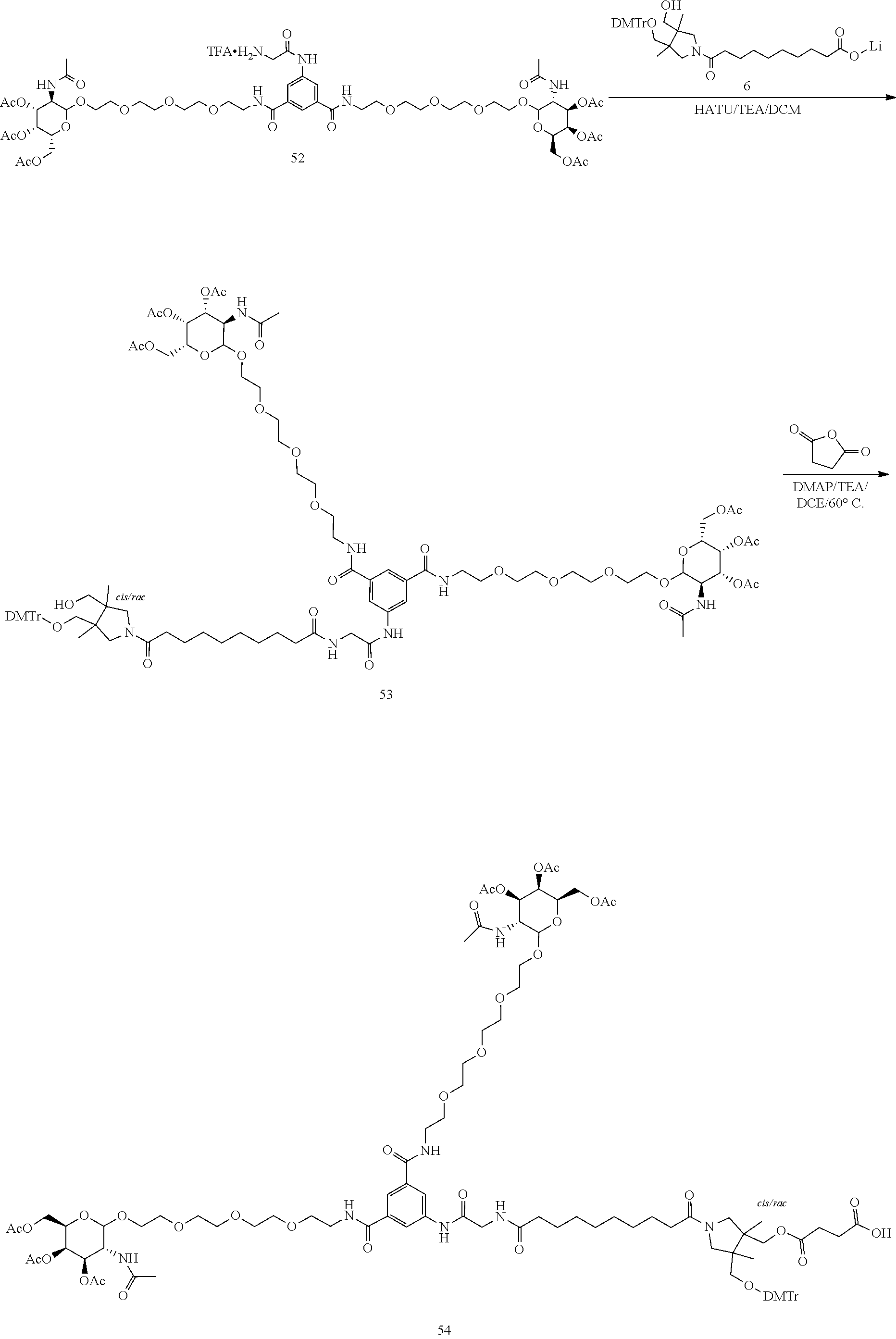

### Step 1. Preparation of Peracetylated Galactosamine 44

D-Galactosamine hydrochloride (14) (250 g, 1.16 mol) in pyridine (1.5 L) was treated with acetic anhydride (1.25 L, 13.2 mol) over 45 minutes. After stirring overnight the reaction mixture was divided into three 1 L portions. Each 1 L portion was poured into 3 L of ice water and mixed for one hour. After mixing the solids were filtered off, combined, frozen over liquid nitrogen and then lyophilized for five days to yield peracetylated galactosamine (44) (369.4 g, 82%) as a white solid. Rf (0.58, 10% MeOH—CH₂Cl₂).

### Step 2. Preparation of (2R,3R,4R,5R,6R)-5-acetamido-2-(acetoxymethyl)-6-(2-(2-(2-(2-azidoethoxy)ethoxy)ethoxy)ethoxy)tetrahydro-2H-pyran-3,4-diyl diacetate 45

Peracetylated galactosamine (44) (25 g, 64.21 mmol) was heated with scandium triflate (1.58 g, 3.21 mmol) in dry DCE at 90° C. for 3 hours. The reaction was cooled to RT, quenched with 5 ml TEA and concentrated in-vacuo. The residue was purified by automated column chromatography (2-10% MeOH/DCM) to give (2R,3R,4R,5R)-5-acetamido-2-(acetoxymethyl)-6-(2-(2-(2-(2-azidoethoxy)ethoxy)ethoxy)ethoxy)tetrahydro-2H-pyran-3,4-diyl diacetate (45) (27 g, 76.5%). Product confirmed by MS.

### Step 3. Preparation of 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy)ethoxy)ethan-1-aminium 2,2,2-trifluoroacetate 46

A solution of the azide 45 (7.12 g, 13 mmol) in EtOAc (150 ml) and trifluoroacetic acid (2 ml) was treated with palladium on charcoal (1.5 g, 10% w/w wet basis). The reaction mixture was then purged with hydrogen and stirred vigorously overnight. After purging with nitrogen, the mixture was filtered through Celite, rinsing with MeOH. The filtrate was concentrated and purified via chromatography (5%→10%→20% MeOH—CH₂Cl₂) to afford 46 (5.8 g, 72%) as a brown oil. Rf (0.34, 15% MeOH—CH₂Cl₂).

### Step 4. Preparation of (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate 48

(2R,3R,4R,5R)-5-Acetamido-2-(acetoxymethyl)-6-((1,1,1-trifluoro-2-oxo-6,9,12-trioxa-3l4-azatetradecan-14-yl)oxy)tetrahydro-2H-pyran-3,4-diyl diacetate (46) (13.25 g, 20.84 mmol), 5-nitroisophthalic acid (47) (2.0 g, 9.5 mmol), HATU (12.3 g, 32.21 mmol) and TEA (5.75 g, 59.0 mmol) were stirred in DCM at RT for 16 h. The reaction was diluted with DCM, washed sequentially with 1M HCl, saturated NaHCO₃, water and brine, dried over Na₂SO₄ and concentrated in-vacuo. The residue was purified by automated flash chromatography (5% MeOH/DCM) to give (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (48) (4.43 g, 38.3%). Product confirmed by MS (ESI +ve).

### Step 5. Preparation of (2R,2′R,3R,3′R,4R,4′R,5R,5′R)-(((5-amino-1,3-phenylene)bis(1-oxo-5,8,11-trioxa-2-azatridecane-1,13-diyl))bis(oxy))bis(5-acetamido-2-(acetoxymethyl)tetra hydro-2H-pyran-6,3,4-triyl) tetraacetate 49

(2R,2′R,3R,3′R,4R,4′R,5R,5′R)-(((5-Nitro-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (48) (26.1 g, 23.05 mmol) was stirred in MeOH at RT. The reaction was hydrogenated over 10% Pd—C (2.6 g) at RT for 2 hours. The reaction was filtered through celite and concentrated in-vacuo to give (2R,2′R,3R,3′R,4R,4′R,5R,5′R)-(((5-amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (49) (28.0 g, 99.9%) which was used in subsequent reactions without further purification. Product confirmed by MS (ESI +ve).

### Step 6. Preparation of (2R,3R,4R,5R)-5-acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy) ethoxy)ethoxy)ethyl)carbamoyl)-5-(2-(((benzyloxy)carbonyl)amino) acetamido)phenyl)-1-oxo-5,8,11-trioxa-2-azatridecan-13-yl)oxy)-2-(acetoxymethyl)tetrahydro-2H-pyran-3,4-diyl diacetate 51

(2R,2′R,3R,3′R,4R,4′R,5R,5′R)-(((5-Amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (49) (0.5 g, 0.45 mmol) and CBZ-gly (50) (0.09 g, 0.45 mmol) were stirred in EtOAc at RT. T3P (50% solution in EtOAc) (0.29 g, 0.91 mmol) was added and the reaction stirred at RT O/N. Additional T3P (0.3 eq) added and the reaction stirred for a further 1 h. The reaction was washed with saturated NaHCO₃ and brine, dried (Na₂SO₄), concentrated in-vacuo and the residue purified by automated flash chromatography (10% MeOH/DCM) to give (2R,3R,4R,5R)-5-acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy)ethoxy)-ethoxy)ethyl)carbamoyl)-5-(2-(((benzyloxy)carbonyl)amino)acetamido)phenyl)-1-oxo-5,8,11-trioxa-2-azatridecan-13-yl)oxy)-2-(acetoxymethyl)tetrahydro-2H-pyran-3,4-diyl diacetate (51) (0.33 g, 56.8%). Product confirmed by MS (ESI +ve).

### Step 7. Preparation of (2R,2′R,3R,3′R,4R,4′R,5R,5′R)-(((5-(2-((2,2,2-trifluoroacetyl)-14-azaneyl)acetamido)-1,3-phenylene)bis(1-oxo-5,8,11-trioxa-2-azatridecane-1,13-diyl))bis (oxy))bis(5-acetamido-2-(acetoxymethyl)tetrahydro-2H-pyran-6,3,4-triyl) tetraacetate 52

(2R,3R,4R,5R)-5-Acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy)ethoxy)ethoxy)ethyl) carbamoyl)-5-(2-(((benzyloxy)carbonyl)amino)acetamido)phenyl)-1-oxo-5,8,11-trioxa-2-azatridecan-13-yl)oxy)-2-(acetoxymethyl)tetrahydro-2H-pyran-3,4-diyl diacetate (51) (3.3 g, 2.39 mmol) and TFA (0.29 g, 2.51 mmol) were stirred in MeOH at RT. The reaction was hydrogenated over 10% Pd—C (400 mg) for two h., filtered through celite and concentrated in-vacuo to give (2R,2′R,3R,3′R,4R,4′R,5R,5′R)-(((5-(2-((2,2,2-trifluoroacetyl)-14-azaneyl)-acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (52) (3.21 g, 98.7%) which was used in subsequent reactions without further purification. Product confirmed by MS (ESI +ve).

### Step 8. Preparation of (2R,2′R,3R,3′R,4R,4′R,5R,5′R)-(((5-(2-(10-(3-((bis(4-methoxyphenyl) (phenyl)methoxy)methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 53

(2R,2′R,3R,3′R,4R,4′R,5R,5′R)-(((5-(2-((2,2,2-Trifluoroacetyl)-14-azaneyl)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (52)(1.0 g, 0.73 mmol), lithium 10-(3-((bis(4-methoxyphenyl)(phenyl)methoxy)methyl)-4-(hydroxymethyl)-3,4-dimethyl-pyrrolidin-1-yl)-10-oxodecanoate (6) (0.45 g, 0.73 mmol), HATU (0.47 g, 1.25 mmol) and TEA (0.22 g, 2.2 mmol) were stirred in DCM at RT for 4 h. The reaction was diluted with DCM and washed sequentially with saturated NaHCO₃, water and brine, dried (Na₂SO₄) and concentrated in-vacuo. The residue was purified by automated flash chromatography (5% MeOH/DCM) to give (2R,2′R,3R,3′R,4R,4′R,5R,5′R)-(((5-(2-(10-(3-((bis(4-methoxy-phenyl)(phenyl)methoxy)methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (53) (1.02 g, 75.2%). Product confirmed by MS (ESI +ve).

### Step 9. Preparation of 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 54

(2R,2′R,3R,3′R,4R,4′R,5R,5′R)-(((5-(2-(10-(3-((Bis(4-methoxyphenyl)(phenyl)methoxy)-methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (54) (1.05 g, 0.57 mmol), succinic anhydride (0.28 g, 2.84 mmol), DMAP (0.35 g, 2.84 mmol) and TEA (0.58 g, 5.68 mmol) were heated in dry DCE at 60° C. for 2 hours. MeOH (5 ml) was added and the reaction stirred for a further 30 mins then cooled and concentrated in-vacuo. The residue was taken up in DCM and washed sequentially with saturated NaHCO₃ (×4), water and brine. The organics were dried (Na₂SO₄), and concentrated in-vacuo to give 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (54) (1.1 g, 99.4%) which was used as a crude product in subsequent reactions. Product confirmed by MS (ESI +ve).

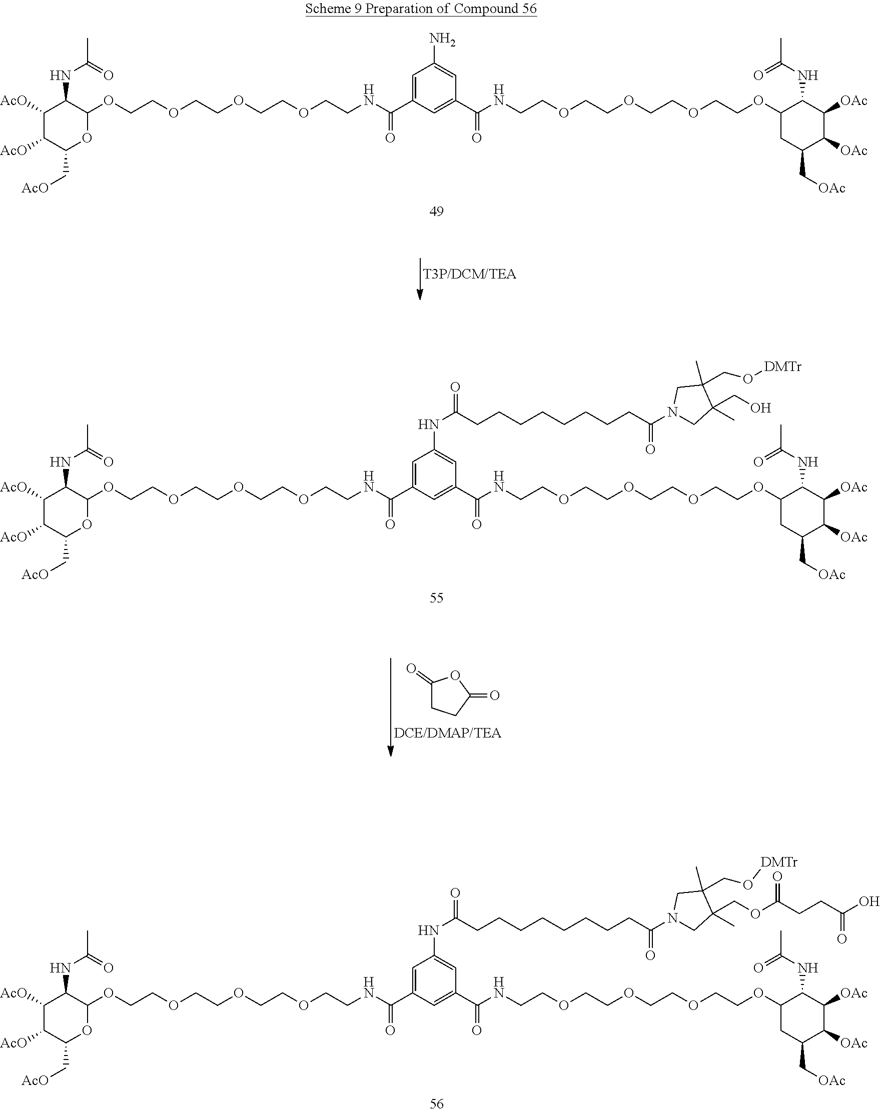

### Step 1. Preparation of (2R,2′R,3R,3′R,4R,4′R,5R,5′R)-(((5-(10-(3-((bis(4-methoxy-phenyl)(phenyl)methoxy)methyl)-4-(hydroxymethyl)-3,4-dimethylpyrrolidin-1-yl)-10-oxodecanamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 55

(2R,2′R,3R,3′R,4R,4′R,5R,5′R)-(((5-Amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (49) (4 g, 3.36 mmol), lithium 10-(3-((bis(4-methoxyphenyl)-(phenyl)methoxy) methyl)-4-(hydroxymethyl)-3,4-dimethylpyrrolidin-1-yl)-10-oxodecanoate (6) (2.13 g, 3.36 mmol), TEA (1 ml, 6.7 mmol) and T3P (50% W/W solution in EtOAc) (4.3 g, 6.72 mmol) were stirred in DCM at RT for 16 h. The reaction was washed sequentially with saturated NaHCO₃, water and brine, dried (Na₂SO₄) and concentrated in-vacuo. The residue was purified by automated flash chromatography (10% MeOH/DCM) to give 2R,2′R,3R,3′R, 4R,4′R,5R,5′R)-(((5-(10-(3-((bis(4-methoxyphenyl)(phenyl)methoxy)methyl)-4-(hydroxyl-methyl)-3,4-dimethylpyrrolidin-1-yl)-10-oxodecanamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (55) (1.37 g, 22.5%). Product confirmed by MS (ESI +ve).

### Step 2. Preparation of 4-((1-(10-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl) carbamoyl)phenyl)amino)-10-oxodecanoyl)-4-((bis(4-methoxyphenyl)(phenyl)methoxy) methyl)-3,4-dimethylpyrrolidin-3-yl)methoxy)-4-oxobutanoic acid 56

This compound was prepared in an analogous manner to 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (54)

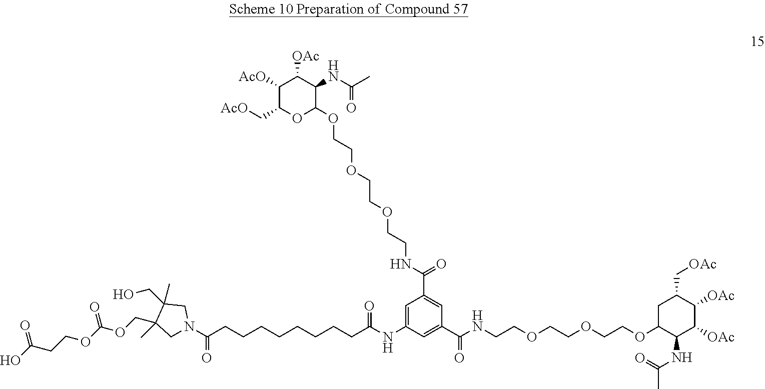 [SMILES: CC(=O)N[C@H]1C(OCCOCCOCCNC(=O)c2cc(NC(=O)CCCCCCCCC(=O)N3CC(C)(CO)C(C)(COC(=O)OCCC(=O)O)C3)cc(C(=O)NCCOCCOCCOC3C[C@H](COC(C)=O)[C@H](OC(C)=O)[C@H](OC(C)=O)[C@H]3NC(C)=O)c2)O[C@H](COC(C)=O)[C@H](OC(C)=O)[C@@H]1OC(C)=O]

### Synthesis of 3-((((1-(10-((3,5-bis((2-(2-(2-(((3R,4R,5R,6R)-3-acetamido-4,5-didicetoxy-6-(acetoxymethyl)tetrahydro-2H-pyran-2-yl)oxy)ethoxy)ethoxy)ethyl)carbamoyl) phenyl)amino)-10-oxodecanoyl)-4-(hydroxymethyl)-3,4-dimethylpyrrolidin-3-yl)methoxy)carbonyl)oxy)propanoic acid 57

This compound was prepared in an analogous manner to 4-((1-(10-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (54)

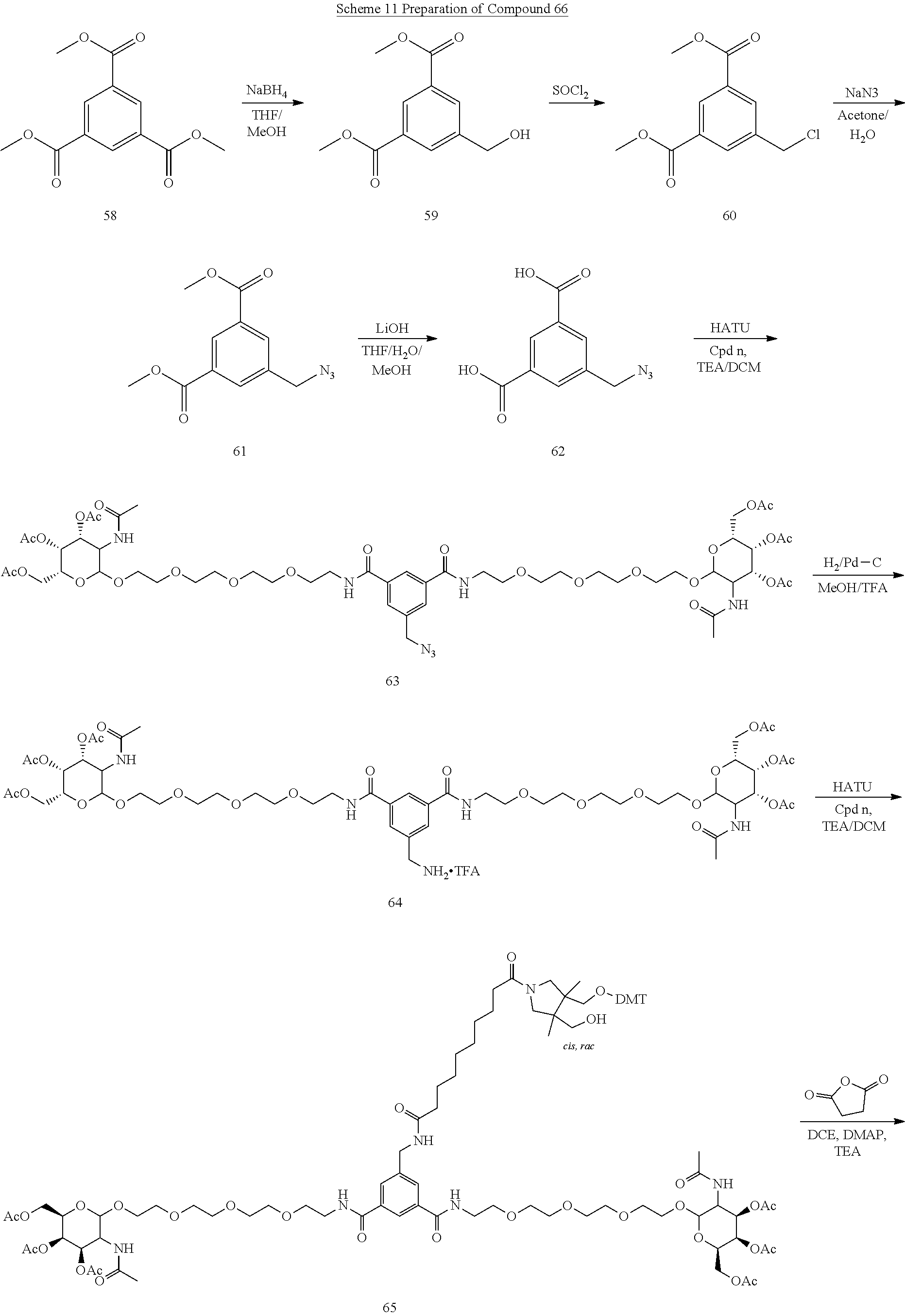

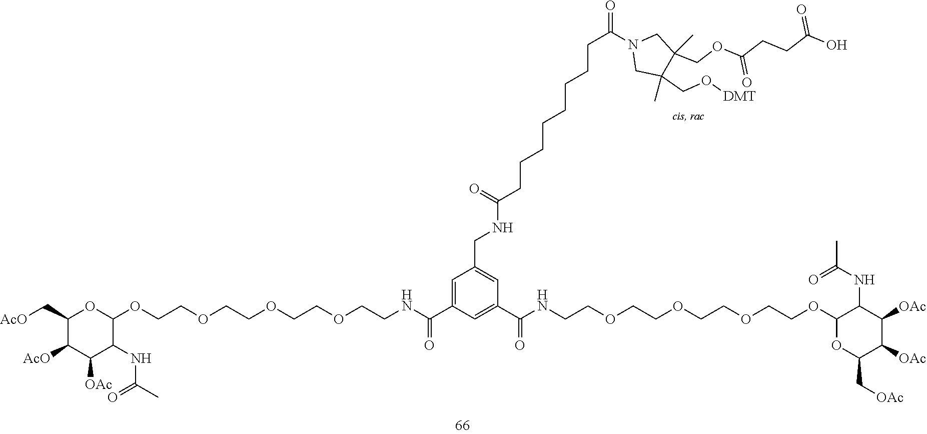

### Step 1. Preparation of dimethyl 5-(hydroxymethyl)isophthalate 59

Trimethyl benzene-1,3,5-tricarboxylate (58) (40 g, 159 mmol) and NaBH₄ were stirred in THF at RT. MeOH (30 ml) in THF (120 ml) was added dropwise slowly. After complete addition the reaction was refluxed for 30 mins. After cooling the reaction was quenched with 1M HCl and extracted into EtOAc. The organics were washed sequentially with 1M HCl, NaHCO₃, water and brine, dried (Na₂SO₄) and concentrated in-vacuo. The residue was purified by automated flash chromatography (50/50 EtOAc/hex) to give dimethyl 5-(hydroxymethyl)isophthalate (59) (20.5 g, 53.2%). ¹H NMR (400 MHz, CDCl₃) δ 8.59 (s, 1H), 8.23 (s, 2H), 4.81 (s, 2H), 3.95 (s, 6H). Product confirmed by MS (ESI +ve).

### Step 2. Preparation of dimethyl 5-(chloromethyl)isophthalate 60

Dimethyl 5-(hydroxymethyl)isophthalate (59) (20.5 g, 80.5%) was refluxed in SOCl₂ (11.1 g, 94 mmol) for 1.5 h. The reaction was cooled, diluted with DCM and washed sequentially with 0.1 M NaOH (×2), water and brine, dried (Na₂SO₄) and concentrated in-vacuo. The residue was purified by automated flash chromatography (20% EtOAc/Hex) to give dimethyl 5-(chloromethyl)isophthalate (60) (10.84 g, 53%). ¹H NMR (400 MHz, CDCl₃) δ 8.65 (s, 1H), 8.27 (s, 2H), 4.66 (s, 2H), 3.97 (s, 6H). Product confirmed by MS (ESI +ve).

### Step 3. Preparation of dimethyl 5-(azidomethyl)isophthalate 61

Dimethyl 5-(chloromethyl)isophthalate (60) (10.84 g, 45 mmol) and NaN₃ (18 g, 270 mmol) were refluxed in acetone/water (3/1) for 16 h. The reaction was cooled, concentrated in-vacuo and the residue taken up in DCM. The organics were washed with water and brine, dried (Na₂SO₄) and concentrated in-vacuo. The residue was purified by flash chromatography (15% EtOAc/Hex) to give dimethyl 5-(azidomethyl)isophthalate (61) (9.84 g, 88%). ¹H NMR (400 MHz, CDCl₃) δ 8.66 (s, 2H), 8.2 (s, 2H), 4.49 (s, 2H), 3.97 (s, 2H). Product confirmed by MS (ESI +ve).

### Step 4. Preparation of 5-(azidomethyl)isophthalic acid 62

Dimethyl 5-(azidomethyl)isophthalate (61) (9.84 g, 39.5 mmol) and LiOH (2.1 g, 87 mmol) were stirred in THF/H₂O/MeOH at RT for 48 h. The organic solvent was removed in-vacuo and the residue acidified with 1M HCl. The aqueous was extracted with EtOAc (×3) and the combined organics dried (Na₂SO₄) and concentrated in-vacuo to give 5-(azidomethyl)isophthalic acid (62) (8.0 g, 91.6%) which was used in subsequent reactions without further purification

### Step 5. Preparation of (2R,2′R,3R,3′R,4R,4′R)-(((5-(azidomethyl)-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate 63

5-(Azidomethyl)isophthalic acid (62) (4.42 g, 20 mmol), 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy) ethoxy)ethan-1-aminium 2,2,2-trifluoroacetate (46) (25 g, 40 mmol), HATU (24.4 g, 64 mmol) and TEA (17 ml, 120 mmol) were stirred in DCM at RT for 16 h. The reaction was washed sequentially with 1M HCl, saturated NaHCO₃, water and brine, dried (Na₂SO₄) and concentrated in-vacuo. The residue was purified by automated flash chromatography (7% MeOH/DCM) to give (2R,2′R,3R,3′R,4R,4′R)-(((5-(azidomethyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (63) (10.9 g, 44.5%). Product confirmed by MS (ESI +ve).

### Step 6. Preparation of (2R,2′R,3R,3′R,4R,4′R)-(((5-(((2,2,2-trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 64

(2R,2′R,3R,3′R,4R,4′R)-(((5-(Azidomethyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (63) (10.9 g, 8.9 mmol) and TFA (0.68 ml, 8.9 mmol) were stirred in MeOH at RT. The reaction was hydrogenated over 10% Pd—C for 1 h. The reaction was filtered through celite, concentrated in-vacuo and the residue purified by automated flash chromatography (15% MeOH/DCM) to give (2R,2′R,3R,3′R,4R,4′R)-(((5-(((2,2,2-trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (64) (6.41 g, 54.7%). Product confirmed by MS (ESI +ve).

### Step 7. Preparation of (2R,2′R,3R,3′R,4R,4′R)-(((5-((10-(3-((bis(4-methoxyphenyl)(phenyl) methoxy)methyl)-4-(hydroxymethyl)-3,4-dimethylpyrrolidin-1-yl)-10-oxodecanamido) methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 65

(2R,2′R,3R,3′R,4R,4′R)-(((5-(((2,2,2-Trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate (3.0 g, 2.3 mmol), lithium 10-(3-((bis(4-methoxyphenyl)(phenyl)methoxy)methyl)-4-(hydroxymethyl)-3,4-dimethylpyrrolidin-1-yl)-10-oxodecanoate (65) (1.5 g, 2.3 mmol), HATU (1.4 g, 3.7 mmol) and TEA (1 ml, 7.0 mmol) were stirred at RT O/N. The reaction was diluted with DCM washed with saturated NaHCO₃, water and brine, dried (Na₂SO₄) and concentrated in-vacuo. The residue was purified by automated flash chromatography (5% MeOH/DCM) to give (2R,2′R,3R,3′R,4R,4′R)-(((5-((10-(3-((bis(4-methoxyphenyl)(phenyl)methoxy)methyl)-4-(hydroxymethyl)-3,4-dimethylpyrrolidin-1-yl)-10-oxodecanamido)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (65) (1.8 g, 43.0%). Product confirmed by MS (ESI +ve).

### Step 8. Preparation of 4-((1-(10-((3,5-bis((2-(2-(2-(2-(((4R,5R,6R)-3-acetamido-4,5-diacetoxy-6-(acetoxy methyl)tetrahydro-2H-pyran-2-yl)oxy)ethoxy)ethoxy)ethoxy)ethyl)carbamoyl)benzyl) amino)-10-oxodecanoyl)-4-((bis(4-methoxyphenyl)(phenyl)methoxy)methyl)-3,4-dimethylpyrrolidin-3-yl)methoxy)-4-oxobutanoic acid 66

This compound was prepared in an analogous manner to 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (54). Product confirmed by MS (ESI +ve).

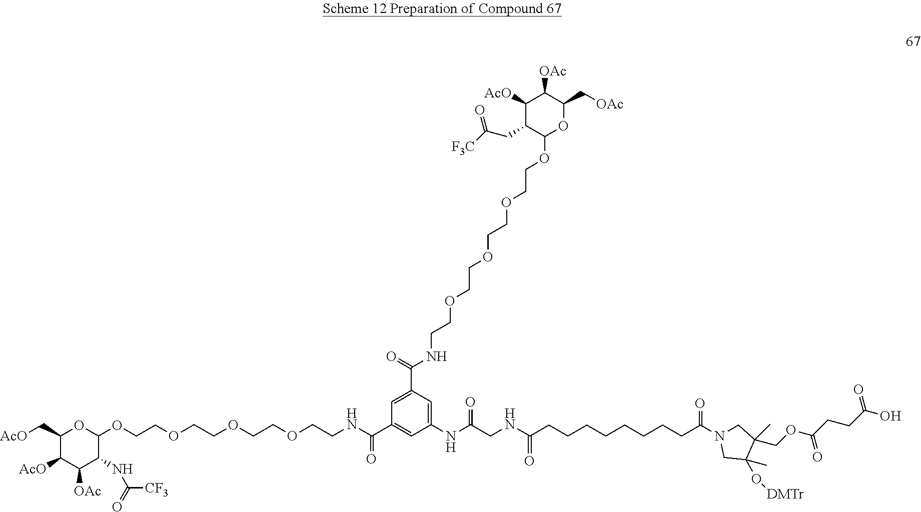

### Synthesis of 4-((1-(10-((2-((3,5-bis((2-(2-(2-(2-(((3R,4R,5R,6R)-4,5-diacetoxy-6-(acetoxy methyl)-3-(2,2,2-trifluoroacetamido)tetrahydro-2H-pyran-2-yl)oxy)ethoxy)ethoxy)ethoxy) ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 67

This compound was prepared in an analogous fashion to 54 (scheme 8), using (3R,4R,5R,6R)-6-(acetoxymethyl)-3-(2,2,2-trifluoroacetamido)tetrahydro-2H-pyran-2,4,5-triyl triacetate instead of peracetylated galactosamine (6). Product confirmed by MS (ESI +ve).

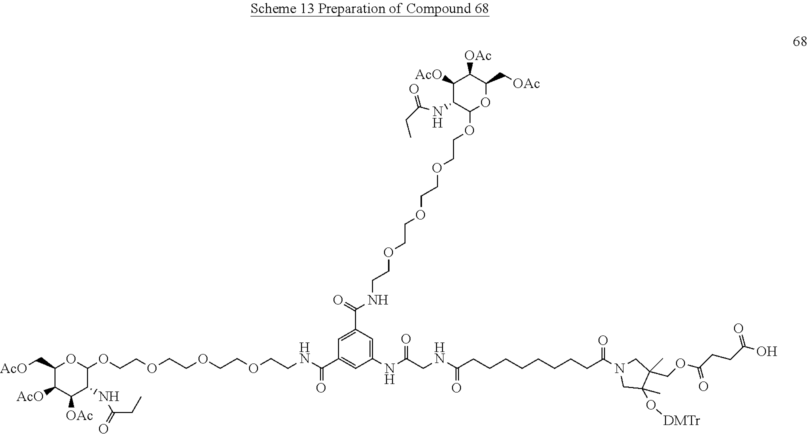

### Synthesis of 4-((1-(10-((2-((3,5-bis((2-(2-(2-(2-(((3R,4R,5R,6R)-4,5-diacetoxy-6-(acetoxy methyl)-3-propionamidotetrahydro-2H-pyran-2-yl)oxy)ethoxy)ethoxy)ethoxy)ethyl) carbamoyl)phenyl)amino)-2-oxoethyl)amino)-10-oxodecanoyl)-4-((bis(4-methoxyphenyl) (phenyl)methoxy)methyl)-3,4-dimethylpyrrolidin-3-yl)methoxy)-4-oxobutanoic acid 68

This compound was prepared in an analogous fashion to 54 (scheme 8), using (3R,4R,5R,6R)-6-(acetoxymethyl)-3-propionamidotetrahydro-2H-pyran-2,4,5-triyl triacetate (19b) instead of peracetylated galactosamine (44). Product confirmed by MS (ESI +ve).

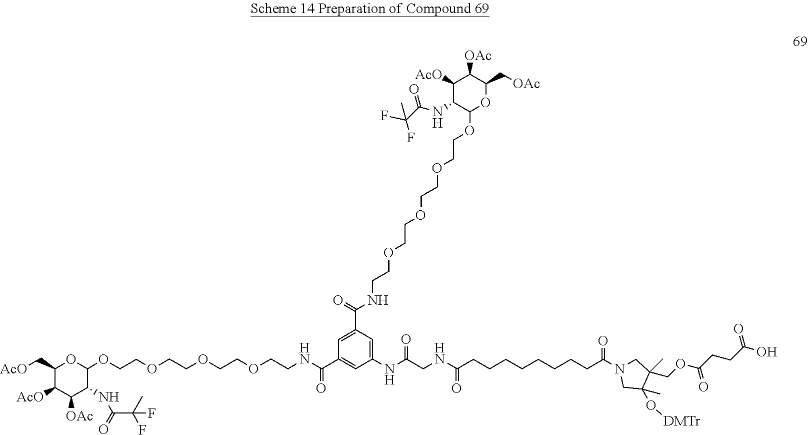

### Synthesis of 4-((1-(10-((2-((3,5-bis((2-(2-(2-(2-(((3R,4R,5R,6R)-4,5-diacetoxy-6-(acetoxy methyl)-3-(2,2-difluoropropanamido)tetrahydro-2H-pyran-2-yl)oxy)ethoxy)ethoxy)-ethoxy) ethyl)carbamoyl)phenyl)amino)-2-oxoethyl)amino)-10-oxodecanoyl)-4-((bis(4-methoxy phenyl)(phenyl)methoxy)methyl)-3,4-dimethylpyrrolidin-3-yl)methoxy)-4-oxobutanoic acid 69

This compound was prepared in an analogous fashion to 54 (scheme 8), using (3R,4R,5R,6R)-6-(acetoxymethyl)-3-(2,2-difluoropropanamido)tetrahydro-2H-pyran-2,4,5-triyl triacetate (19c) instead of peracetylated galactosamine (44). Product confirmed by MS (ESI +ve).

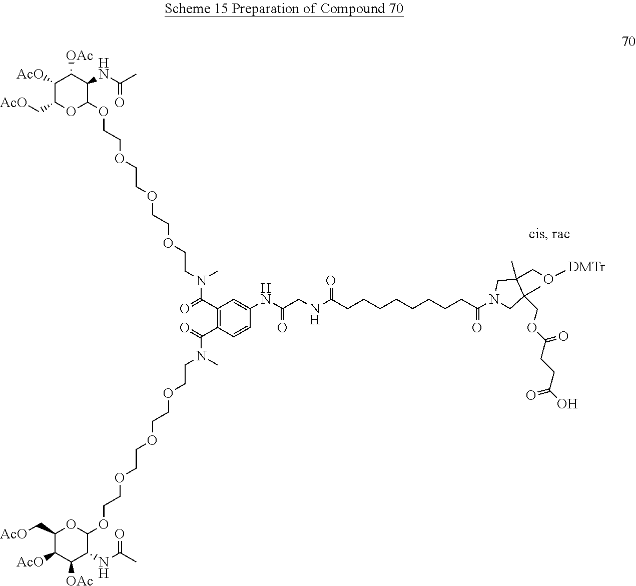

### Synthesis of 4-((1-(10-((2-((3,4-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)(methyl) carbamoyl)phenyl)amino)-2-oxoethyl)amino)-10-oxodecanoyl)-4-((bis(4-methoxyphenyl) (phenyl)methoxy)methyl)-3,4-dimethylpyrrolidin-3-yl)methoxy)-4-oxobutanoic acid 70

This compound was prepared in an analogous manner to compound 54 (scheme 8) using (2R,2′R,3R,3′R,4R,4′R,5R,5′R)-(((4-nitro-1,2-phenylene)bis(2-methyl-1-oxo-5′, 8′, 11′-trioxa-2′-azatridecane-1,13-diyl))bis(oxy))bis(5-acetamido-2-(acetoxymethyl)tetrahydro-2H-pyran-6,3,4-triyl) tetraacetate (34) in place of (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate (48).

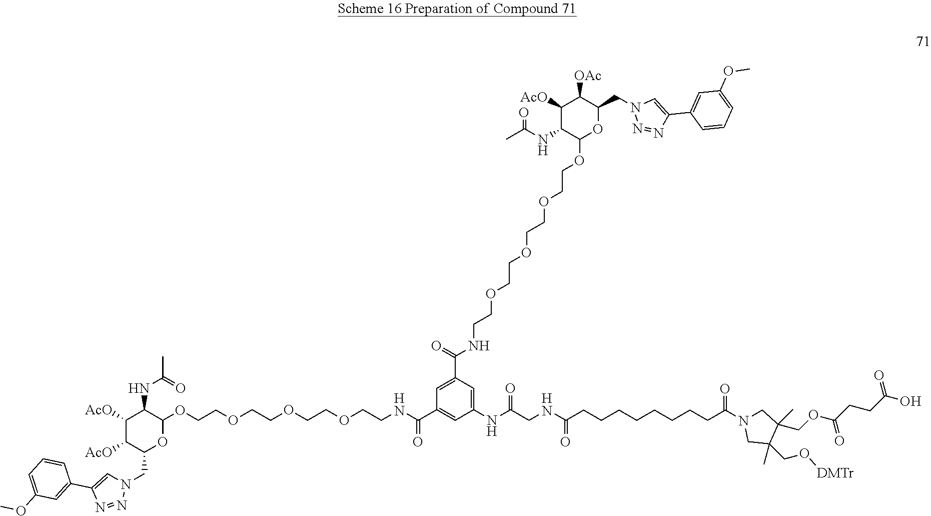

### Synthesis of 4-((1-(10-((2-((3,5-bis((2-(2-(2-(2-(((3R,4R,5S,6R)-3-acetamido-4,5-diacetoxy-6-((4-(3-methoxyphenyl)-1H-1,2,3-triazol-1-yl)methyl)tetrahydro-2H-pyran-2-yl)oxy)ethoxy) ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 71

This compound was prepared in an analogous manner to compound 54 (scheme 8) using (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-3l4-azatetradecan-14-yl)oxy)tetrahydro-2H-pyran-3,4-diyl diacetate (43) in place of 2-(2-(2-(2-(((2R,3R,4R,5R,6R)-3-acetamido-4,5-diacetoxy-6-(acetoxymethyl) tetrahydro-2H-pyran-2-yl)oxy)ethoxy)ethoxy)ethoxy)ethan-1-aminium 2,2,2-trifluoroacetate (46).

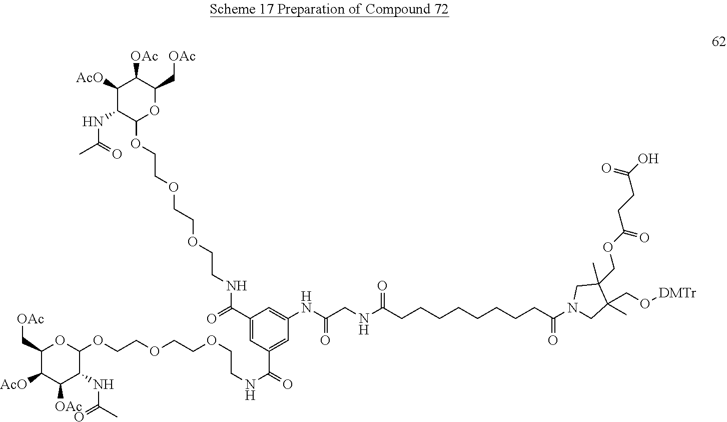

### Synthesis of 4-((4-(10-((2-((3,5-bis((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)phenyl) amino)-2-oxoethyl)amino)-10-oxodecanoyl)-2-((bis(4-methoxyphenyl)(phenyl)methoxy) methyl)-1,2-dimethylcyclopentyl)methoxy)-4-oxobutanoic acid 72

This compound was prepared in an analogous manner to compound 54 (scheme 8) using (2R,3R,4R,5R)-5-acetamido-2-(acetoxymethyl)-6-(2-(2-(2-((2,2,2-trifluoroacetyl)-14-azaneyl)ethoxy) ethoxy)ethoxy)tetrahydro-2H-pyran-3,4-diyl diacetate (24) in place of 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy) ethoxy)ethan-1-aminium 2,2,2-trifluoroacetate (46).

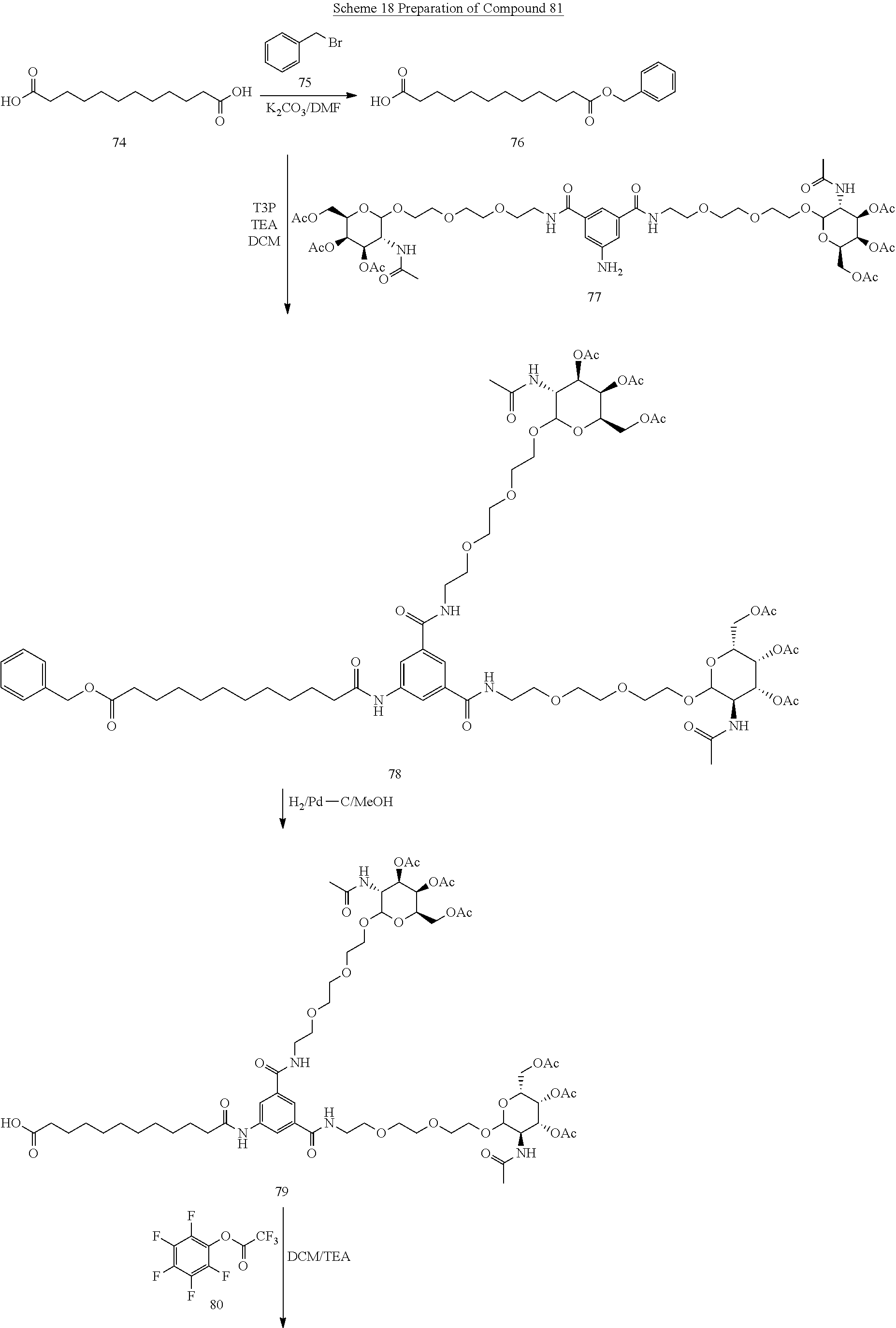

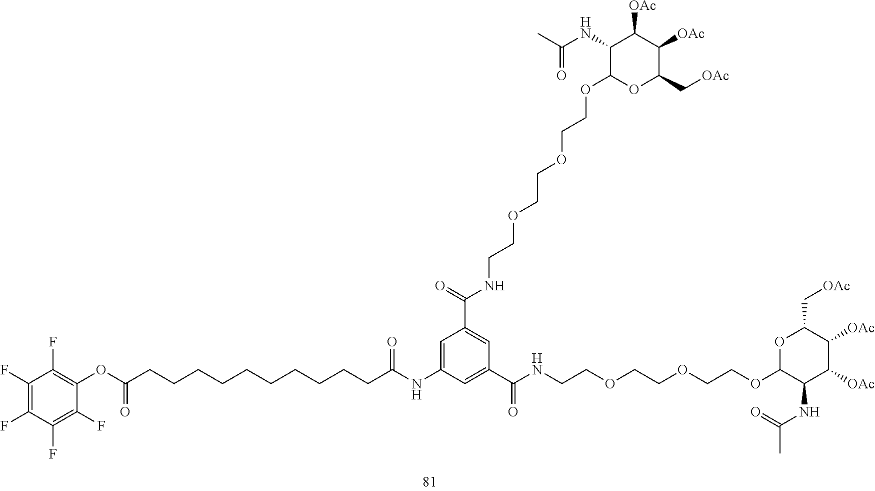 [SMILES: CC(=O)N[C@H]1C(OCCOCCOCCNC(=O)c2cc(NC(=O)CCCCCCCCCCC(=O)Oc3c(F)c(F)c(F)c(F)c3F)cc(C(=O)NCCOCCOCCOC3O[C@H](COC(C)=O)[C@H](OC(C)=O)[C@H](OC(C)=O)[C@H]3NC(C)=O)c2)O[C@H](COC(C)=O)[C@H](OC(C)=O)[C@@H]1OC(C)=O]

### Step 1. Preparation of 12-(benzyloxy)-12-oxododecanoic acid 76

To a solution of dodecanedioic acid (74) (21.0 g, 91.3 mmol) in DMF (200 ml) was added potassium carbonate (10 g, 72.4 mmol) and benzyl bromide (75) (10 ml, 84.2 mmol). The solution was stirred at 80° C. for 4 hours, cooled to 0° C. then carefully acidified with 6M HCl. Dilute with water (250 ml) and extract with ethyl acetate (500 ml). The ethyl acetate extract was washed with brine (3×250 ml), dried on magnesium sulfate, filtered and concentrated to dryness. The solid was suspended in dichloromethane (200 ml) and filtered. The filtrate, which was now enriched in the product, was concentrated then purified by column chromatography on silica gel 60 (Gradient: 0 to 10% methanol in DCM) to afford 12-(benzyloxy)-12-oxododecanoic acid (76) as a colorless solid (13 g, 45%). Structure confirmed by mass spectroscopy

### Step 2. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-(12-(benzyloxy)-12-oxododecanamido)benzamido)ethoxy)ethoxy) ethoxy)-2-(acetoxymethyl)tetrahydro-2H-pyran-3,4-diyl diacetate 78

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy)ethyl) carbamoyl)-5-aminobenzamido)ethoxy)ethoxy)ethoxy)-2-(acetoxymethyl)-tetrahydro-2H-pyran-3,4-diyl diacetate (77) (4.0 g, 3.6 mmol), 12-(benzyloxy)-12-oxododecanoic acid (76) (1.3 g, 4.1 mmol) and triethylamine (1.5 ml, 10.8 mmol) in dichloromethane (75 ml) was added dropwise T3P (4.5 g, ˜9 ml, 50% solution in ethyl acetate). The solution was stirred overnight at room temperature. Upon completion, the reaction mixture was diluted with dichloromethane and carefully quenched with a saturated solution of sodium bicarbonate (200 ml). The biphasic solution was stirred vigorously for 30 minutes. The DCM layer was separated and the aqueous phase was extracted with dichloromethane (1×100 ml). The combined extracts were dried on magnesium sulfate, filtered and concentrated in vacuo to dryness. The residue was purified by column chromatography on silica gel 60 (Gradient: 0-10% MeOH in DCM) to afford the title compound as a colorless solid (1.5 g, 30%).

### Step 3. Preparation of 12-((3-((2-(2-(2-(((3R,4R,5R,6R)-3-acetamido-4,5-diacetoxy-6-(acetoxy methyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-12-oxododecanoic acid 79

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)-ethyl) carbamoyl)-5-(12-(benzyloxy)-12-oxododecanamido)benzamido)ethoxy)ethoxy)-ethoxy)-2-(acetoxymethyl)tetrahydro-2H-pyran-3,4-diyl diacetate (78) (1.5 g, 1.1 mmol) in methanol (25 ml) was added 10% palladium on carbon (wet basis, 150 mg, 10% wt/wt). The solution was sparged with hydrogen gas slowly over 1 hour. Upon completion, the solution was sparged with nitrogen, filtered through celite, and concentrated in vacuo to dryness to afford a colorless solid (1.1 g, 79%).

### Step 4. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(12-oxo-12-(perfluorophenoxy)dodecanamido)benzamido)ethoxy) ethoxy)ethoxy)-2-(acetoxymethyl)tetrahydro-2H-pyran-3,4-diyl diacetate 81

To a solution of 12-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl) carbamoyl)phenyl)amino)-12-oxododecanoic acid (79) (0.6 g, 0.46 mmol) and triethylamine (125 μL, 0.92 mmol) in dichloromethane (50 ml) was added pentafluorophenyl trifluoroacetate (80) (150 mg, 1.1 mmol). The solution was stirred for 30 minutes at room temperature then concentrated in vacuo to dryness. The residue was purified by column chromatography on silica gel 60 (gradient: 0 to 10% methanol in dichloromethane) to afford the title compound as a colorless solid (475 mg, 70%). Mass (ESI+) m/z 741.0 (M+2H). 1H NMR (400 MHz, DMSO-d6) δ 10.12 (s, 1H), 8.52 (t, J=5.6 Hz, 2H), 8.14 (d, J=1.4 Hz, 2H), 7.91 (t, J=1.6 Hz, 1H), 7.80 (d, J=9.2 Hz, 2H), 5.21 (d, J=3.4 Hz, 2H), 4.97 (dd, J=11.2, 3.4 Hz, 2H), 4.54 (d, J=8.5 Hz, 2H), 4.06-3.99 (m, 7H), 3.88 (dt, J=11.2, 8.8 Hz, 2H), 3.77 (ddd, J=11.1, 5.6, 3.9 Hz, 2H), 3.62-3.46 (m, 22H), 3.46-3.38 (m, 5H), 2.77 (t, J=7.2 Hz, 2H), 2.31 (t, J=7.4 Hz, 2H), 2.10 (s, 7H), 1.99 (s, 7H), 1.89 (s, 7H), 1.77 (s, 7H), 1.69-1.54 (m, 4H), 1.40-1.20 (m, 14H). Mass (ESI+) m/z 741.0 (M+2H).

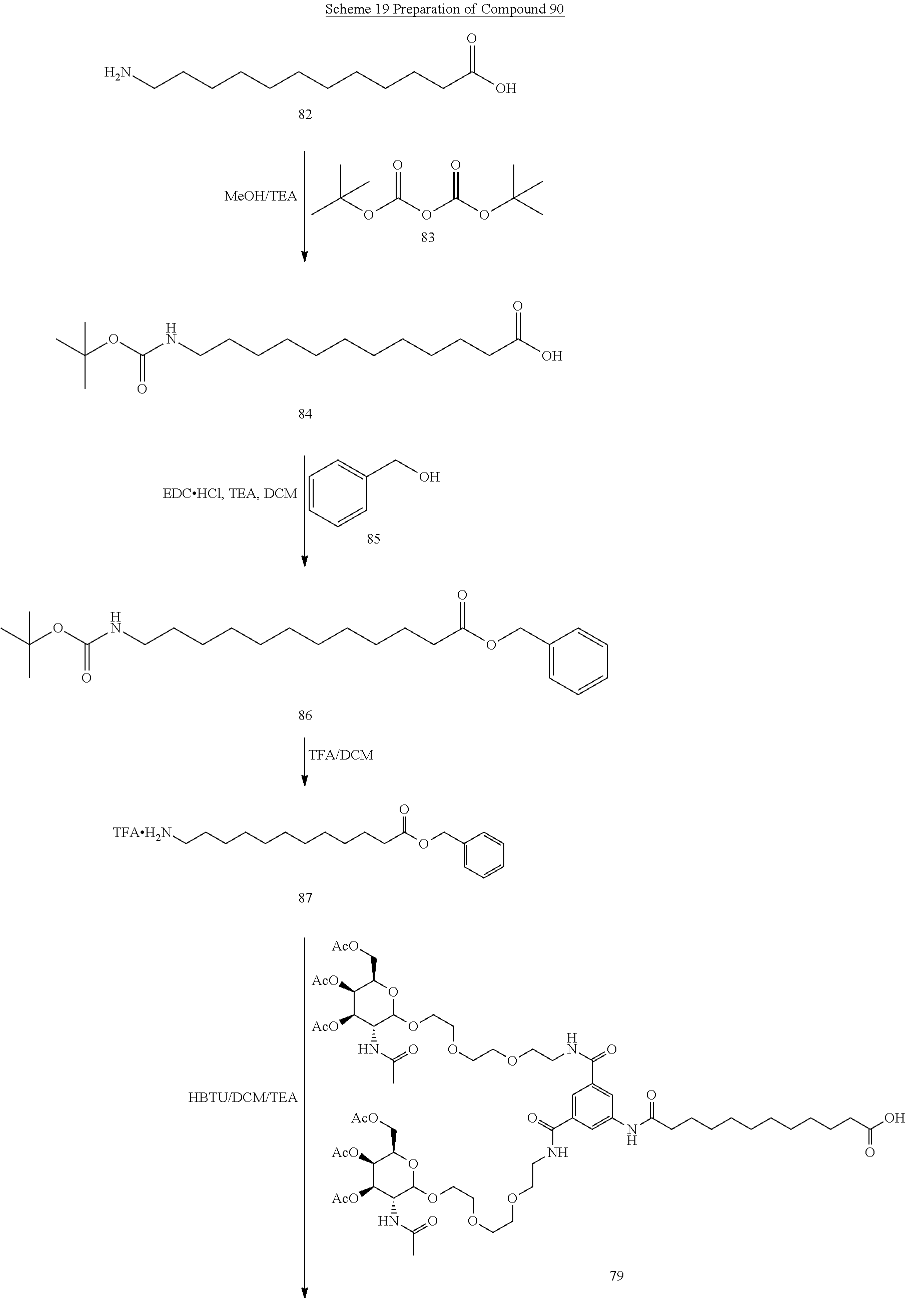

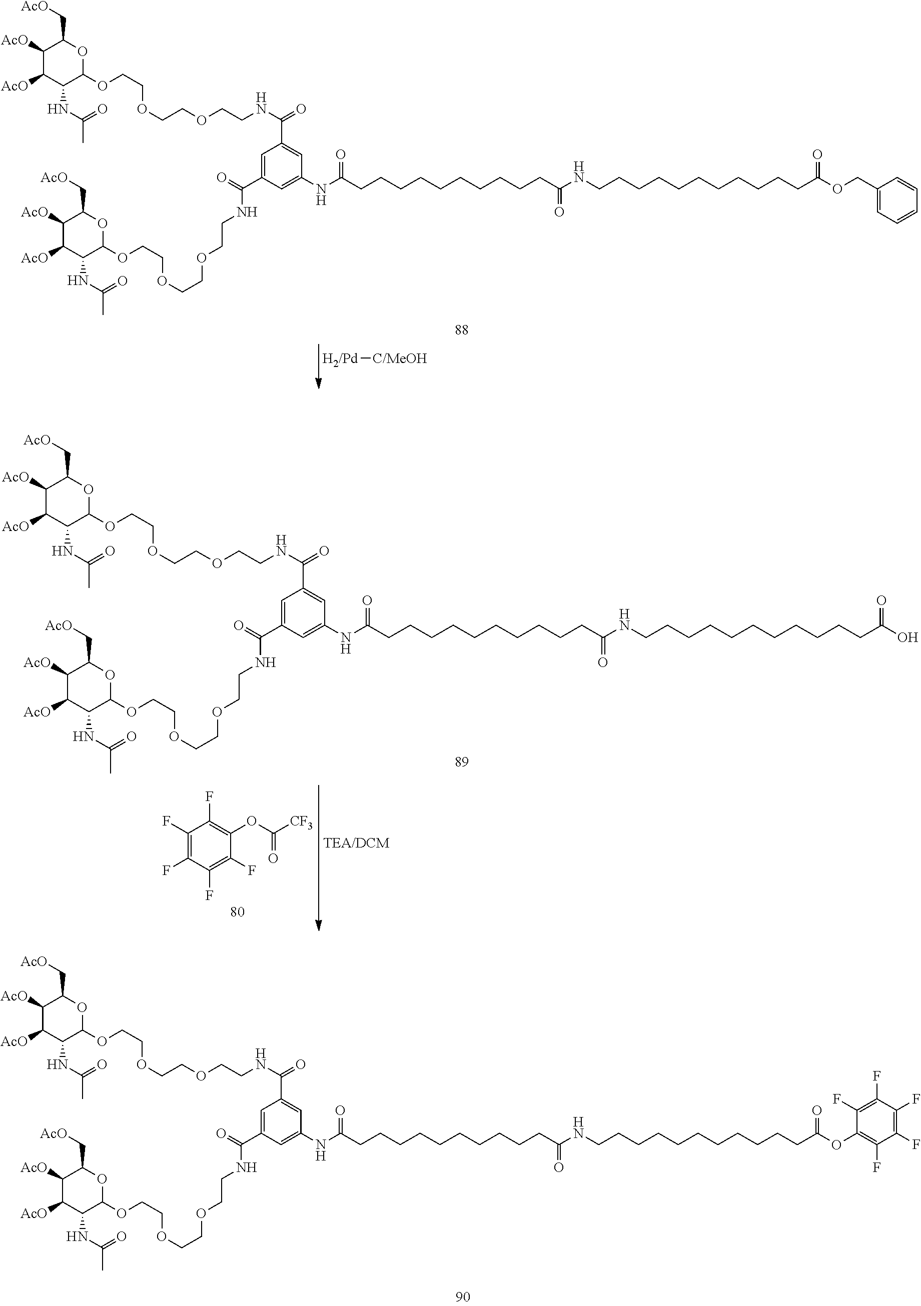

### Step 1. Preparation of 12-((tert-butoxycarbonyl)amino)dodecanoic acid 84

A solution of 12-aminododecanoic acid (82) (5.0 g, 23.3 mmol), di-tert-butyl decarbonate (83) (6.1 g, 27.9 mmol) and triethylamine (6.3 ml, 46.6 mmol) in methanol (75 ml) was heated to 60° C. for 3 h then at room temperature overnight. Upon completion, the solution was concentrated in vacuo to dryness and used in the next step without further purification.

### Step 2. Preparation of benzyl 12-((tert-butoxycarbonyl)amino)dodecanoate 85

A solution of crude 12-((tert-butoxycarbonyl)amino)dodecanoic acid (84) (9.0 g, 30.0 mmol), benzyl alcohol (85) (3.1 g, 30.0 mmol), EDC hydrochloride (6.9 g, 36.0 mmol) and triethylamine (12 ml, 90.0 mmol) in dichloromethane (100 ml) was stirred at room temperature overnight. Upon completion, the solution was washed with saturated sodium bicarbonate solution (100 ml) and brine (100 ml). The dichloromethane solution was dried on magnesium sulfate, filtered and concentrated to dryness. Purification by column chromatography on silica gel 60 (Gradient: 0 to 50% ethyl acetate in hexanes) afforded the title compound as a colorless solid (2.0 g, 21% over two steps).

### Step 3. Preparation of 12-(benzyloxy)-12-oxododecan-1-aminium trifluoroacetate 87

A solution of benzyl 12-((tert-butoxycarbonyl)amino)dodecanoate (86) (2.0 g, 4.9 mmol), dichloromethane (15 ml) and TFA (5 ml) was stirred overnight at room temperature. The reaction mixture was concentrated to dryness to afford the product as a viscous oil (2.1 g. quantitative).

### Step 4. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(12-((12-(benzyloxy)-12-oxododecyl)amino)-12-oxododecanamido) benzamido)ethoxy)ethoxy)ethoxy)-2-(acetoxymethyl)tetrahydro-2H-pyran-3,4-diyl diacetate 88

A solution of 12-((3-((2-(2-(2-(((3R,4R,5R,6R)-3-acetamido-4,5-diacetoxy-6-(acetoxymethyl) tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)-ethyl) carbamoyl)phenyl)amino)-12-oxododecanoic acid (88) (750 mg, 0.54 mmol), 12-(benzyloxy)-12-oxododecan-1-aminium trifluoroacetate (87) (225 mg, 0.54 mmol), HBTU (210 mg, 0.54 mmol) and diisopropylethylamine (0.3 ml, 1.62 mmol) in dichloromethane (30 ml) was stirred overnight at room temperature. The solution was diluted with dichloromethane (50 ml) and washed with saturated bicarbonate solution (100 ml). The dichloromethane was dried on magnesium sulfate, filtered and concentrated in vacuo to dryness. The residue was purified by column chromatography on silica gel 60 (gradient: 0 to 10% methanol in dichloromethane) to afford the title compound (88) as a colorless solid (605 mg, 70%).

### Step 5. Preparation of 12-(12-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-12-oxododecanamido)dodecanoic acid 89

Hydrogenation was conducted as previously described to give (89) (350 mg, 55%)

### Step 6. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)-ethoxy)ethoxy) ethyl)carbamoyl)-5-(12-oxo-12-((12-oxo-12-(perfluorophenoxy)-dodecyl)amino) dodecanamido)benzamido)ethoxy)ethoxy)ethoxy)-2-(acetoxymethyl)-tetrahydro-2H-pyran-3,4-diyl diacetate 90

PFP ester formation was conducted as described previously to give the required product (90) (112 mg, 23%). ¹H NMR (400 MHz, DMSO-d₆) δ 10.12 (s, 1H), 8.91 (s, 1H), 8.65 (t, J=5.5 Hz, 1H), 8.52 (t, J=5.6 Hz, 1H), 8.23 (d, J=1.5 Hz, 1H), 8.14 (t, J=1.4 Hz, 2H), 7.91 (d, J=1.6 Hz, 1H), 7.80 (d, J=9.2 Hz, 2H), 7.68 (t, J=5.6 Hz, 1H), 5.21 (d, J=3.4 Hz, 2H), 4.97 (dd, J=11.2, 3.4 Hz, 2H), 4.54 (d, J=8.5 Hz, 2H), 4.07-3.96 (m, 6H), 3.88 (dt, J=11.2, 8.9 Hz, 2H), 3.81-3.74 (m, 2H), 3.64-3.36 (m, 24H), 3.15-3.03 (m, 6H), 2.99 (q, J=6.5 Hz, 2H), 2.76 (t, J=7.2 Hz, 1H), 2.31 (t, J=7.4 Hz, 1H), 2.10 (s, 6H), 1.99 (s, 7H), 1.89 (s, 7H), 1.76 (s, 6H), 1.70-1.53 (m, 3H), 1.47 (q, J=7.1 Hz, 2H), 1.40-1.10 (m, 29H). Mass (ESI+) m/z 839.7 (M+2H).

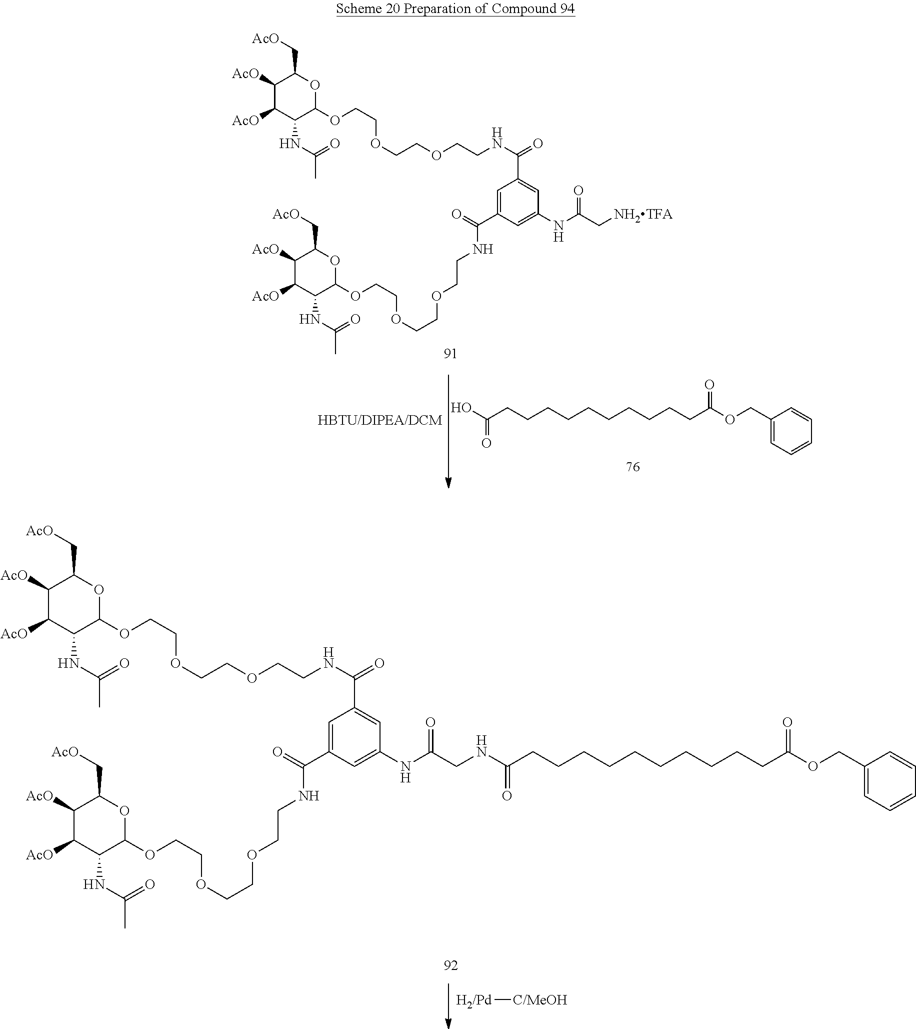

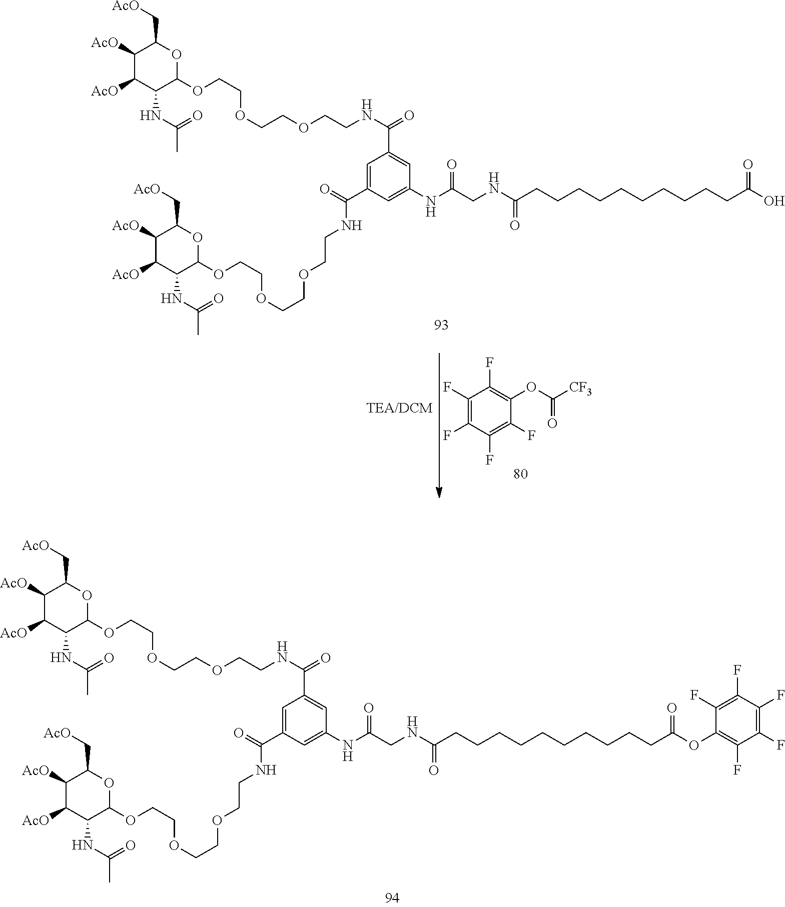 [SMILES: CC(=O)N[C@H]1C(OCCOCCOCCNC(=O)c2cc(NC(=O)CNC(=O)CCCCCCCCCCC(=O)O)cc(C(=O)NCCOCCOCCOC3O[C@H](COC(C)=O)[C@H](OC(C)=O)[C@H](OC(C)=O)[C@H]3NC(C)=O)c2)O[C@H](COC(C)=O)[C@H](OC(C)=O)[C@@H]1OC(C)=O ; O=C(Oc1c(F)c(F)c(F)c(F)c1F)C(F)(F)F ; CC(=O)N[C@H]1C(OCCOCCOCCNC(=O)c2cc(NC(=O)CNC(=O)CCCCCCCCCCC(=O)Oc3c(F)c(F)c(F)c(F)c3F)cc(C(=O)NCCOCCOCCOC3O[C@H](COC(C)=O)[C@H](OC(C)=O)[C@H](OC(C)=O)[C@H]3NC(C)=O)c2)O[C@H](COC(C)=O)[C@H](OC(C)=O)[C@@H]1OC(C)=O]

### Step 1. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(2-(12-(benzyloxy)-12-oxododecanamido)acetamido)benzamido)ethoxy) ethoxy)ethoxy)-2-(acetoxymethyl)tetrahydro-2H-pyran-3,4-diyl diacetate 92

A solution of 2-((3-((2-(2-(2-(((3R,4R,5R,6R)-3-acetamido-4,5-diacetoxy-6-(acetoxymethyl) tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)-ethoxy)ethyl) carbamoyl)phenyl)amino)-2-oxoethan-1-aminium trifluoroacetate (91) (1.0 g, 0.8 mmol), 12-(benzyloxy)-12-oxododecanoic acid (76) (256 mg, 0.8 mmol), HBTU (341 mg, 0.9 mmol) and diisopropylethylamine (0.4 ml, 2.4 mmol) in dichloromethane (20 ml) was stirred overnight at room temperature. Upon completion, the reaction mixture was diluted with dichloromethane (80 ml) and washed with saturated sodium bicarbonate (100 ml). The solution was dried on magnesium sulfate, filtered and concentrated in vacuo to dryness. The residue was purified by column chromatography on silica gel 60 (gradient: 0 to 10% methanol in dichloromethane) to afford the title compound as a colorless solid (0.8 g, 68%).

### Step 2. Preparation of 12-((2-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-2-oxoethyl)amino)-12-oxododecanoic acid 93

Compound 93 was prepared using conditions similar to those described herein for a similar conversion (450 mg, 60%).

### Step 3. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy)ethyl)carbamoyl)-5-(2-(12-oxo-12-(perfluorophenoxy)dodecanamido)acetamido) benzamido)ethoxy)ethoxy)ethoxy)-2-(acetoxymethyl)tetrahydro-2H-pyran-3,4-diyl diacetate 94

Compound 94 was prepared using conditions similar to those described herein for a similar conversion (460 mg, 91%). Mass (ESI+) m/z 1537.8 (M+H).

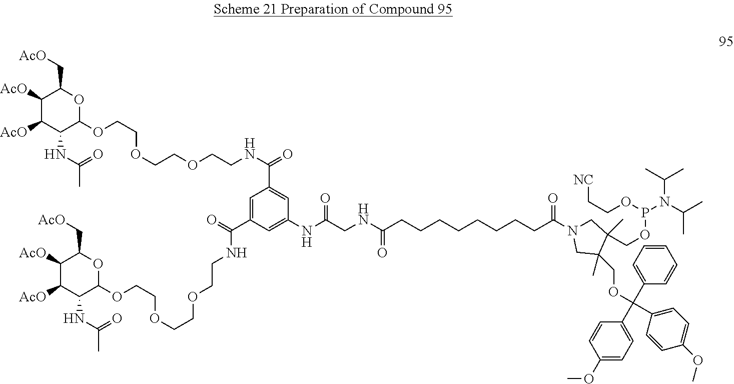 [SMILES: COc1ccc(C(OCC2(C)CN(C(=O)CCCCCCCCC(=O)NCC(=O)Nc3cc(C(=O)NCCOCCOCCOC4O[C@H](COC(C)=O)[C@H](OC(C)=O)[C@H](OC(C)=O)[C@H]4NC(C)=O)cc(C(=O)NCCOCCOCCOC4O[C@H](COC(C)=O)[C@H](OC(C)=O)[C@H](OC(C)=O)[C@H]4NC(C)=O)c3)CC2(C)COP(OCCC#N)N(C(C)C)C(C)C)(c2ccccc2)c2ccc(OC)cc2)cc1]

### Synthesis of (2R,2′R,3R,3′R,4R,4′R,5R,5′R)-((((((((5-(2-(10-(3-((bis(4-methoxyphenyl) (phenyl)methoxy)methyl)-4-((((2-cyanoethoxy)(diisopropylamino)phosphaneyl)-oxy)methyl)-3,4-dimethylpyrrolidin-1-yl)-10-oxodecanamido)acetamido)-isophthaloyl)bis(azanediyl))bis (ethane-2,1-diyl))bis(oxy))bis(ethane-2,1-diyl))bis(oxy))-bis(ethane-2,1-diyl))bis(oxy))bis(5-acetamido-2-(acetoxymethyl)tetrahydro-2H-pyran-6,3,4-triyl) tetraacetate 95

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)-ethyl) carbamoyl)-5-(2-(10-(3-((bis(4-methoxyphenyl)(phenyl)methoxy)methyl)-4-(hydroxymethyl)-3,4-dimethylpyrrolidin-1-yl)-10-oxodecanamido)acetamido)benzamido)-ethoxy)ethoxy)ethoxy)-2-(acetoxymethyl)tetrahydro-2H-pyran-3,4-diyl diacetate (72) (1.6 g, 0.9 mmol) and diisopropylethylamine (0.4 ml, 1.8 mmol) in anhydrous dichloromethane (25 ml) was added 2-Cyanoethyl N,N-diisopropylchlorophosphoramidite (0.3 ml, 1.35 mmol). The solution was stirred for 75 minutes at room temperature then concentrated to dryness. The residue was purified by column chromatography (gradient: 0 to 10% MeOH in DCM (0.1% TEA)) to afford the product as a colorless solid (1.1 g, 62%). 31P NMR (400 MHz, DMSO-d6): δ 146.76 (s), 146.42 (s, 2 overlapping signals), 146.34 (s). 1H NMR (400 MHz, DMSO-d6) δ 10.20 (s, 1H), 8.54 (t, J=5.6 Hz, 2H), 8.17-8.09 (m, 3H), 7.94 (s, 1H), 7.80 (d, J=9.2 Hz, 2H), 7.39-7.26 (m, 4H), 7.26-7.17 (m, 6H), 6.91-6.83 (m, 4H), 5.21 (d, J=3.4 Hz, 2H), 4.97 (dd, J=11.2, 3.4 Hz, 2H), 4.54 (d, J=8.5 Hz, 2H), 4.02 (s, 6H), 3.93-3.82 (m, 4H), 3.73 (s, 10H), 3.66-3.36 (m, 35H), 3.28-3.06 (m, 6H), 3.06-2.87 (m, 3H), 2.72-2.63 (m, J=11.5, 5.8 Hz, 2H), 2.10 (m, 12H), 1.99 (s, 6H), 1.89 (s, 6H), 1.77 (s, 6H), 1.47 (d, J=7.2 Hz, 4H), 1.23 (dq, J=13.9, 6.4 Hz, 18H), 1.17-1.04 (m, 10H), 0.98 (dt, J=13.4, 5.9 Hz, 10H).

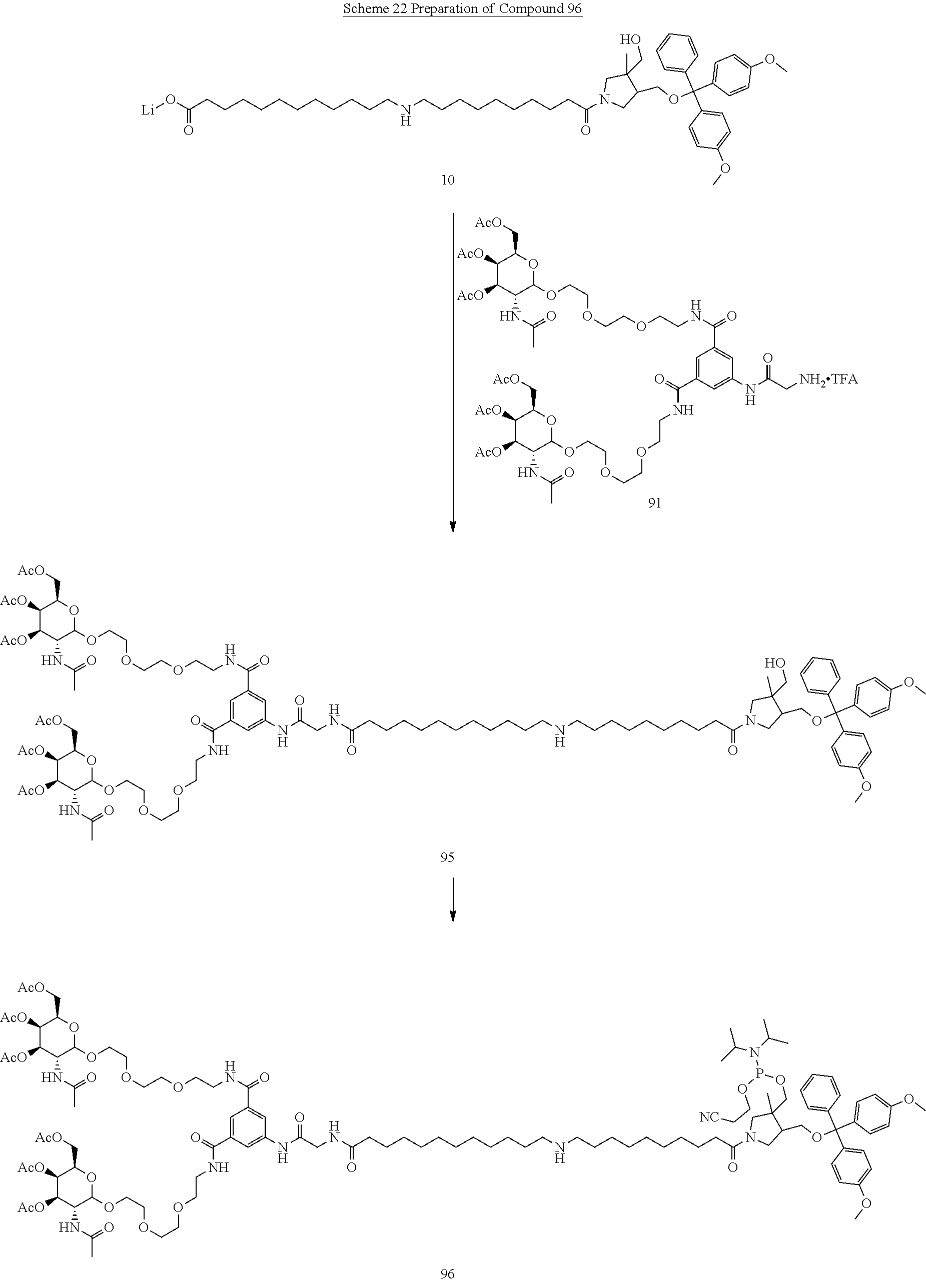

### Step 1. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(2-(12-((10-(3-((bis(4-methoxyphenyl)(phenyl)methoxy)methyl)-4-(hydroxymethyl)-3,4-dimethylpyrrolidin-1-yl)-10-oxodecyl)amino)dodecanamido) acetamido)benzamido)ethoxy)ethoxy)ethoxy)-2-(acetoxymethyl)tetrahydro-2H-pyran-3,4-diyl diacetate 95

Compound 95 was prepared using conditions similar to those described herein for a similar conversion (1.9 g, 61%).

### Step 2: Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy) ethyl)carbamoyl)-5-(2-(12-((10-(3-((bis(4-methoxyphenyl)(phenyl)methoxy)-methyl)-4-((((2-cyanoethoxy)(diisopropylamino)phosphaneyl)oxy)methyl)-3,4-dimethylpyrrolidin-1-yl)-10-oxodecyl)amino)dodecanamido)acetamido)benzamido)-ethoxy)ethoxy)ethoxy)-2-(acetoxymethyl)tetrahydro-2H-pyran-3,4-diyl diacetate 96

Compound 96 was prepared using conditions similar to those described herein for a similar conversion (1.35 g, 65%). ³¹P NMR (400 MHz, DMSO-d₆): δ 146.79 (s), 146.76 (s), 146.42 (s), 146.36 (s). ¹H NMR (400 MHz, DMSO-d₆) δ 10.19 (s, 1H), 8.54 (t, J=5.6 Hz, 2H), 8.13 (dd, J=6.1, 3.5 Hz, 3H), 7.94 (s, 1H), 7.80 (d, J=9.2 Hz, 2H), 7.71-7.65 (m, 1H), 7.39-7.25 (m, 4H), 7.25-7.17 (m, 4H), 6.92-6.83 (m, 4H), 5.21 (d, J=3.4 Hz, 2H), 4.97 (dd, J=11.2, 3.4 Hz, 2H), 4.54 (d, J=8.5 Hz, 2H), 4.07-3.97 (m, 6H), 3.94-3.82 (m, 4H), 3.82-3.74 (m, 2H), 3.73 (s, 6H), 3.62-3.45 (m, 23H), 3.42 (m, 6H), 3.27-2.92 (m, 14H), 2.73-2.62 (m, 2H), 2.10 (s, 8H), 1.99 (s, 9H), 1.89 (s, 6H), 1.77 (s, 6H), 1.52-1.42 (m, 6H), 1.22 (d, J=8.0 Hz, 24H), 1.17 (t, J=7.3 Hz, 11H), 1.09 (dt, J=6.7, 3.3 Hz, 9H), 1.03-0.92 (m, 9H).

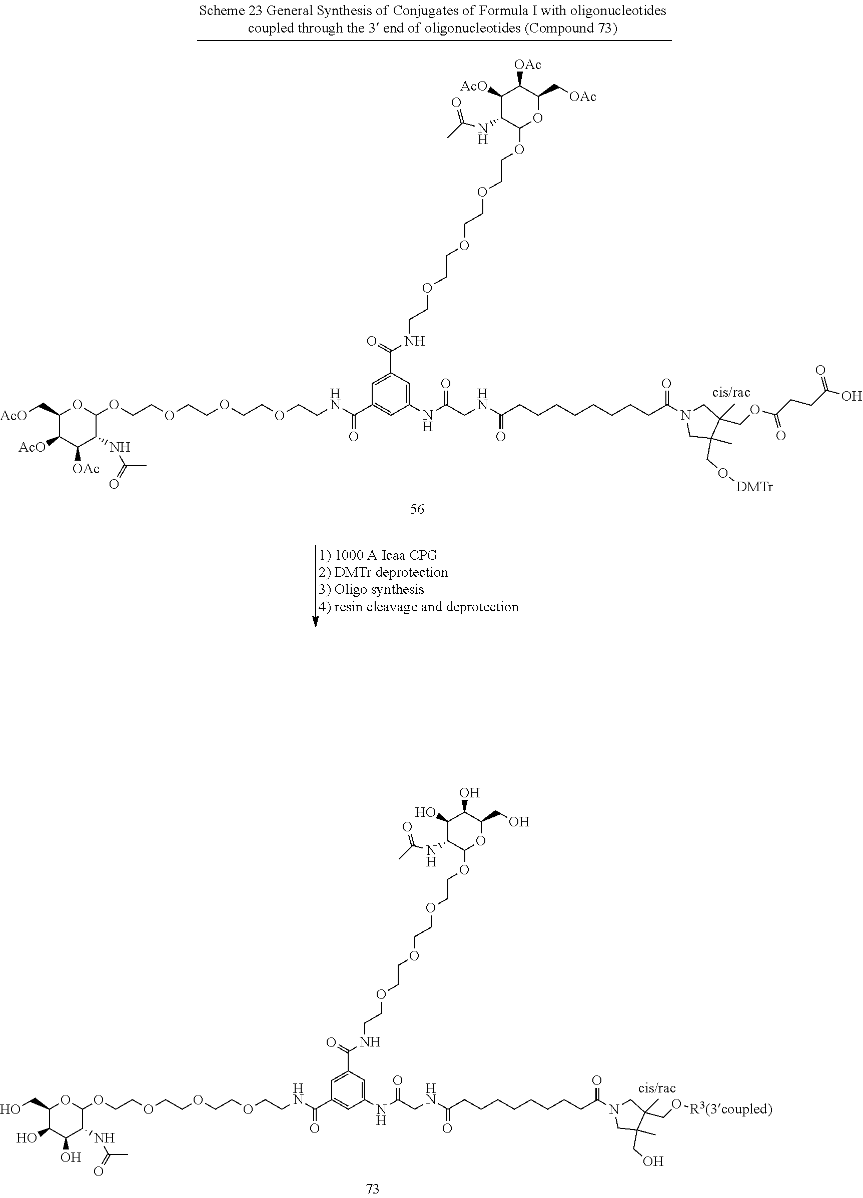

### General Method for Synthesizing Bidentate ASGPr Targeting Ligands from Succinate Ligands Exemplified for 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)-ethyl) carbamoyl)phenyl)amino)-2-oxoethyl)amino)-10-oxodecanoyl)-4-((bis(4-methoxyphenyl) (phenyl)methoxy)methyl)-3,4-dimethylpyrrolidin-3-yl)methoxy)-4-oxobutanoic acid 97

The succinate was loaded onto 1000A LCAA (long chain aminoalkyl) CPG (control pore glass) using standard amide coupling chemistry. LCAA CPG (2.0 g) was suspended in DCM (5 ml) and MeCN (7.6 ml). Diisopropylcarbodiimide (100 μl), N-hydroxy succinimide (110 μl, 30 μM/g), pyridine (110 μL) and 56 (200 mg, 0.1 mmol) were added and the suspension was gently mixed for 16 h at RT. The CPG was recovered by filtration, washed with DCM (×3) and MeCN (×3) and dried under high vacuum. A solution of 5% acetic anhydride/5% N-methylimidazole/5% pyridine in THF was added and the suspension agitated at RT for 2 h. The CPG was recovered by filtration, washed with DCM (×3) and MeCN (×3) and dried under high vacuum. Loading was determined to be 31.3 μmol/g (DMTr assay by UV/Vis 504 nm). The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 97.

### Schemes 24 and 25 General Synthesis of Conjugates of Formula I with Oligonucleotides Coupled Through the 5′ End of Oligonucleotides (Compound 98)

Pentafluorophenyl esters were coupled to a C₆ 5′-amino modifier with phosphate/phosphorothioate linkage on the sense strand oligonucleotide using standard coupling conditions. Standard cleavage and deprotection afforded the desired sense strand conjugate. For example the pentafluorophenyl ester 81 was used to afford the conjugate 98 below.

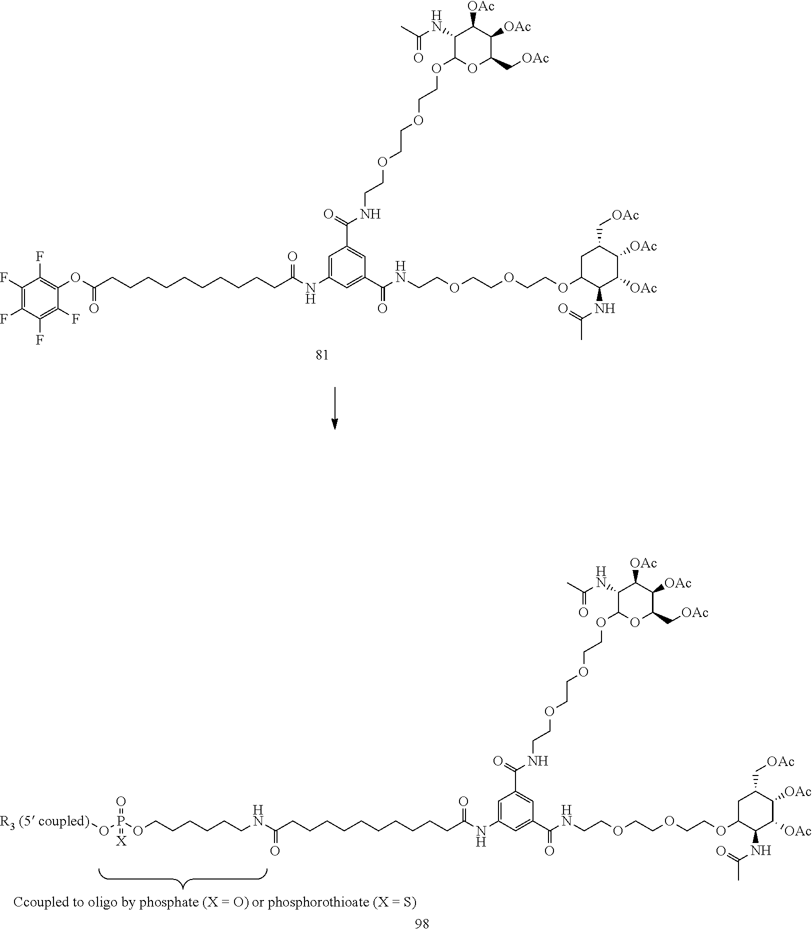

Phosphoramidites were coupled to the 5′ hydroxyl of the sense strand terminal nucleotide using standard phosphoramidite coupling chemistry. Standard cleavage and deprotection afforded the desired sense strand conjugate. For example phosphoramidite 95 was used to afford the conjugate 99 below.

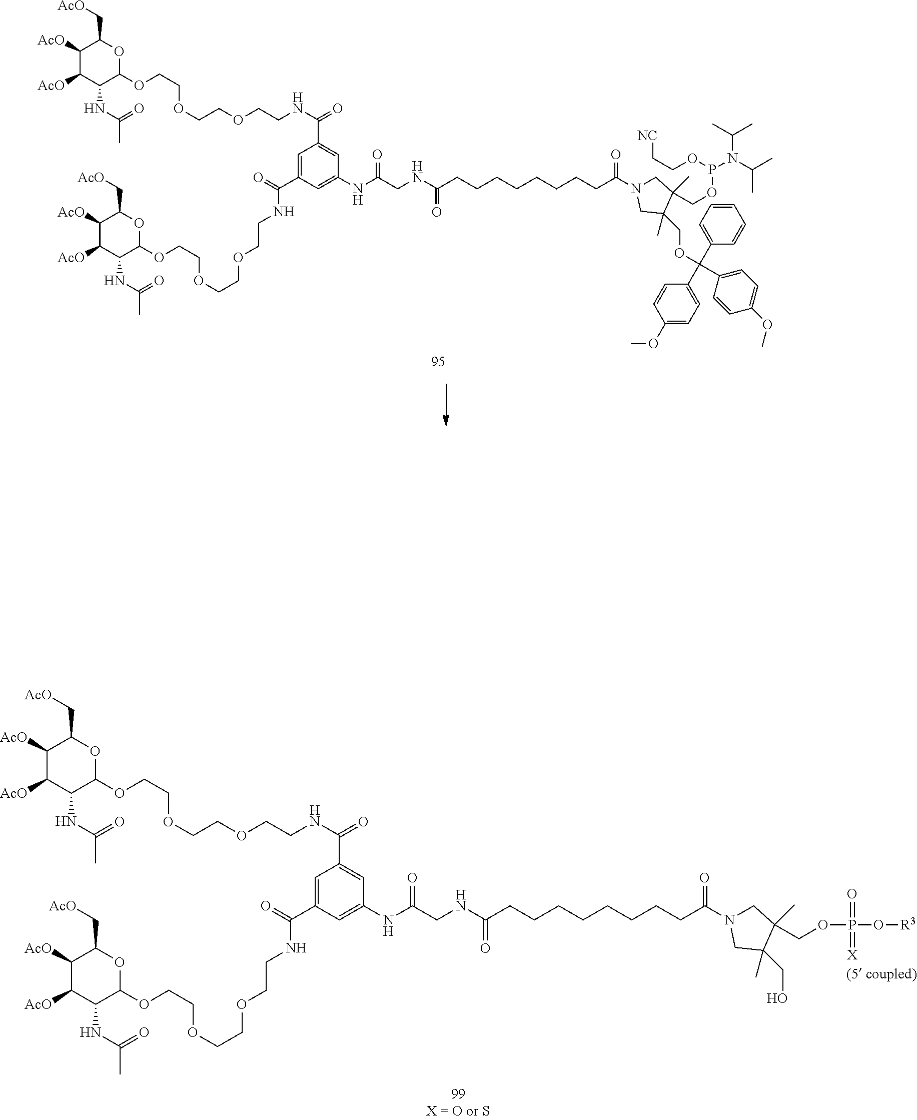

### Examples 1-9

Using the general procedure illustrated in Scheme 23, the following conjugates of the invention were prepared, wherein R^{3b} is the modified TTR siRNA described in Table A below. Masses are for single strand products—not the annealed duplex.

### Example 1

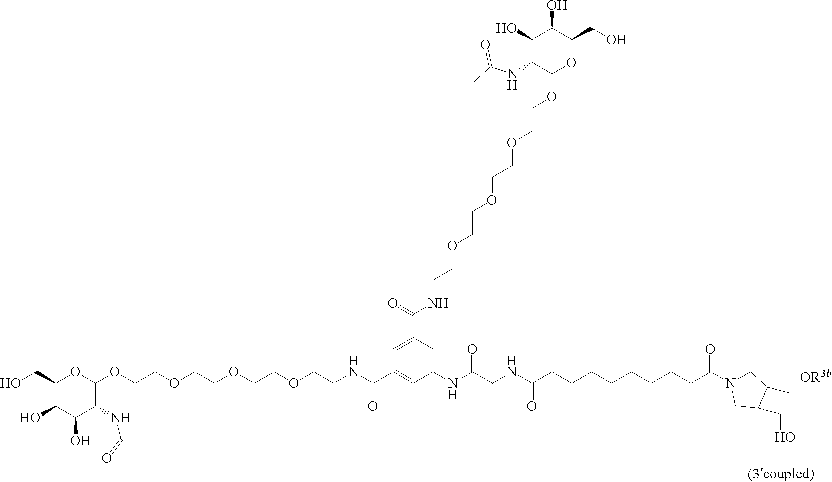

MS (+VE) calculated: 8184.7; measured: 8184.2

### Example 2

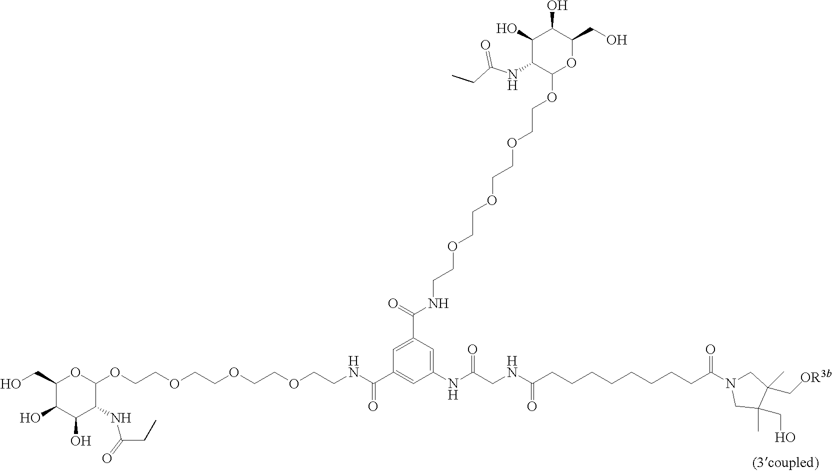

MS (+VE) calculated: 8212.7; measured: 8211.9

### Example 3

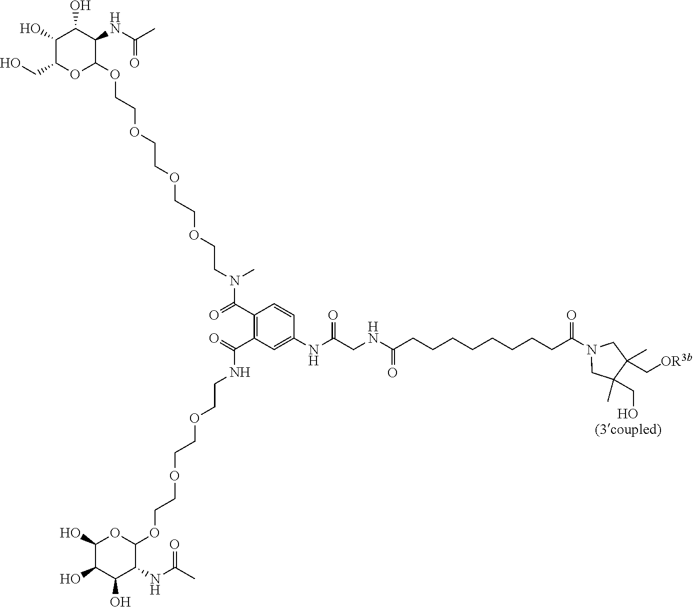

MS (+VE) calculated: 8212.7; measured: 8212.8

### Example 4

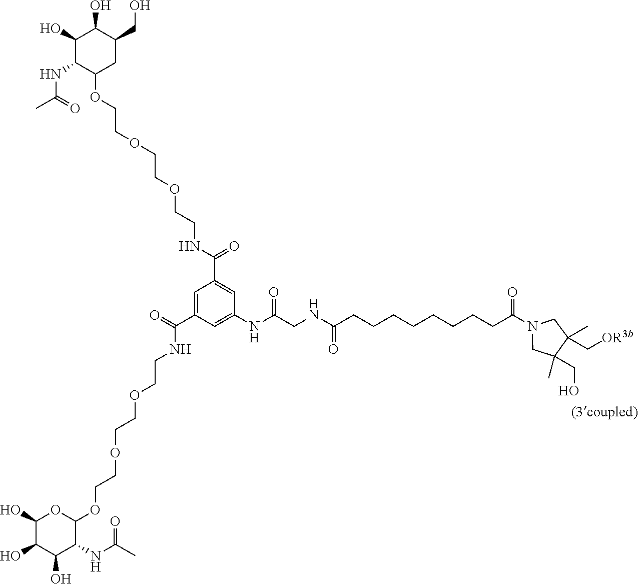

MS (+VE) calculated: 8096.6; measured: 8097.0

### Example 5

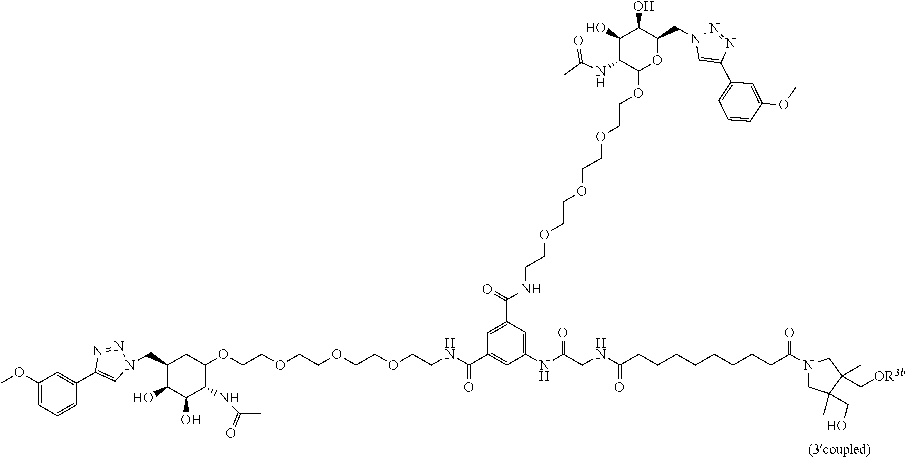

MS (+VE) calculated: 8499.0; measured: 8498.7

### Example 6

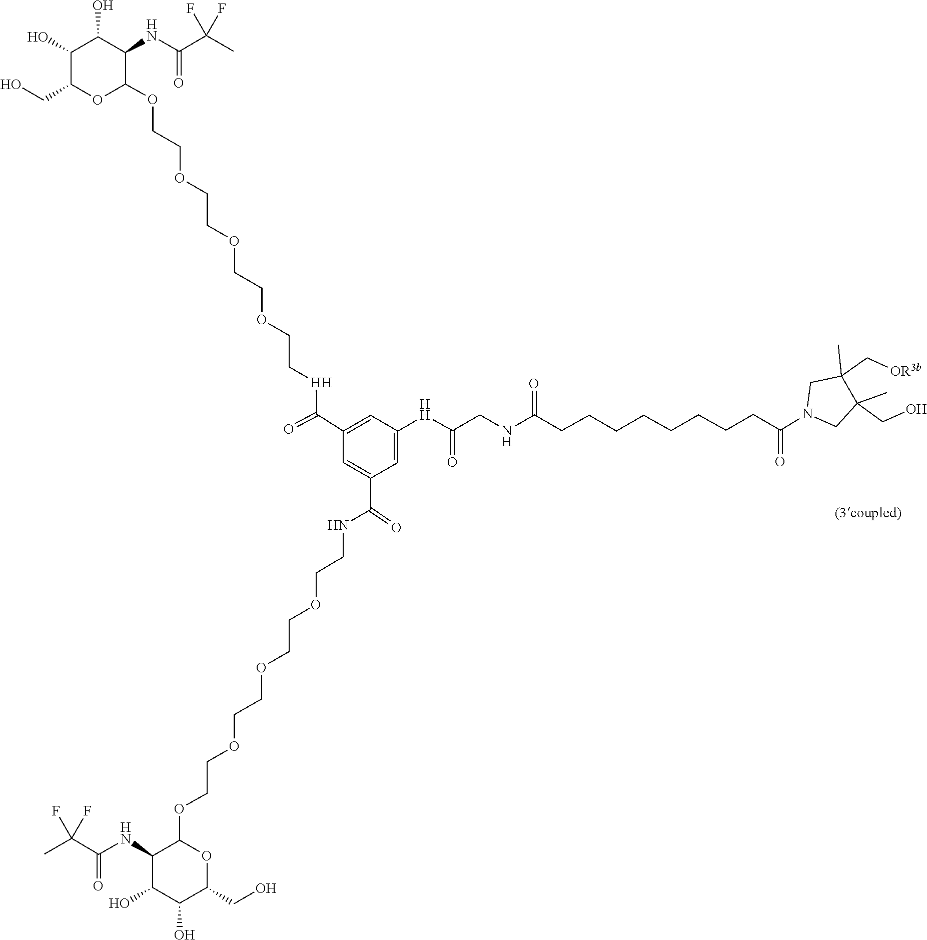

MS (+VE) calculated: 8284.7; measured: 8283.8

### Example 7

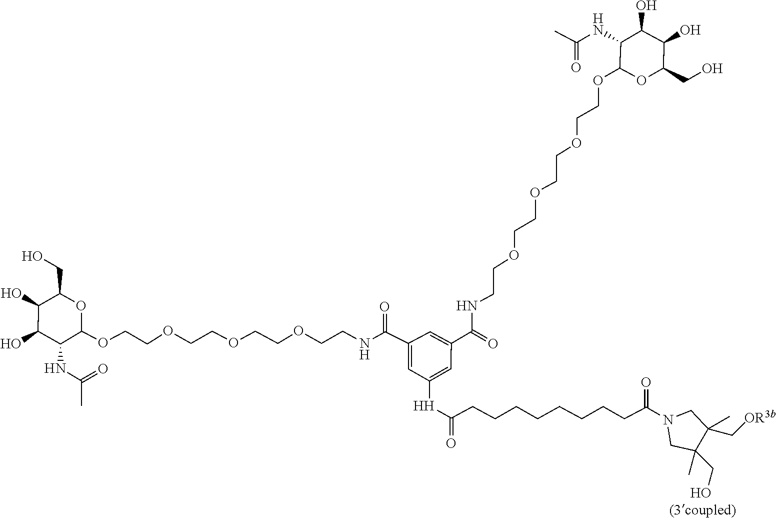

MS (+VE) calculated: 7596.0; measured: 7596.8

### Example 8

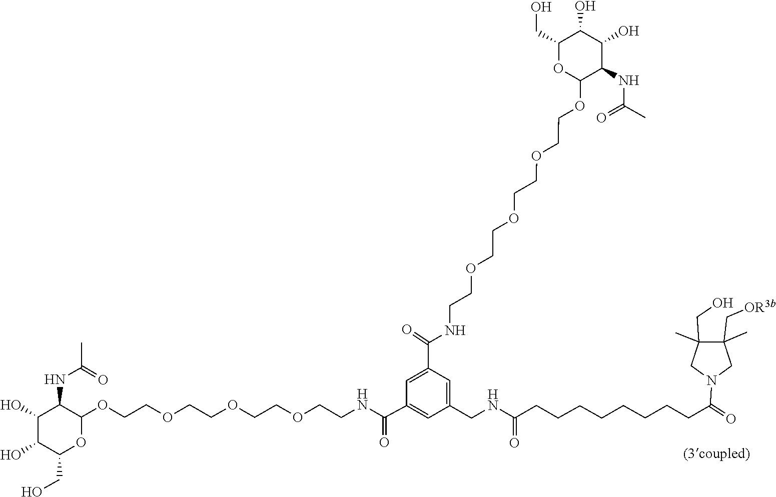

MS (+VE) calculated: 8140.6; measured: 8139.6

### Example 9

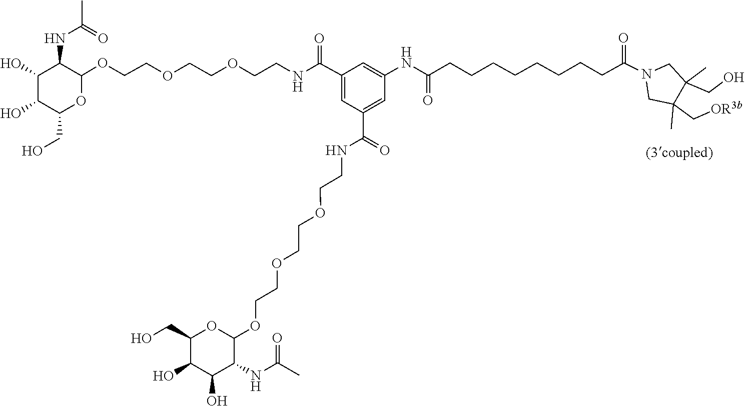

MS (+VE) calculated: 8038.5; measured: 8037.5

### Examples 10-11

Using the general procedure illustrated in Scheme 24, the following conjugates of the invention were prepared, wherein R^{3b} is the modified TTR siRNA described in Table A below. Masses are for single strand products—not the annealed duplex.

### Example 10

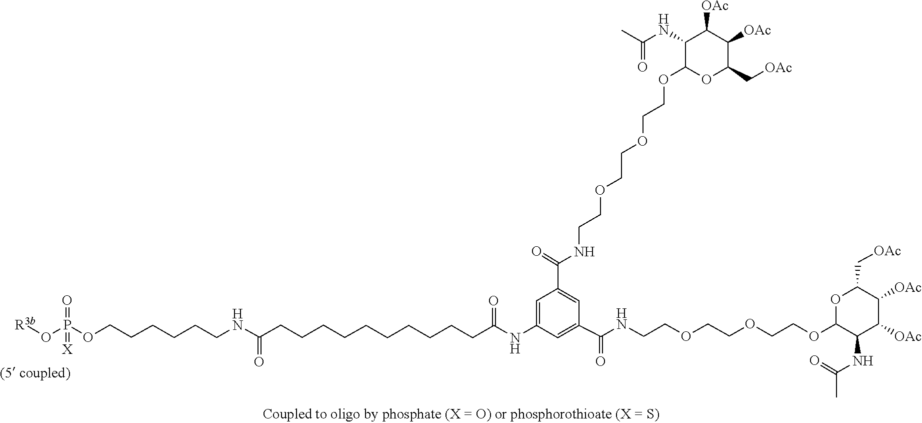

MS (+VE) calculated: 8056.7; measured: 8056.1

### Example 11

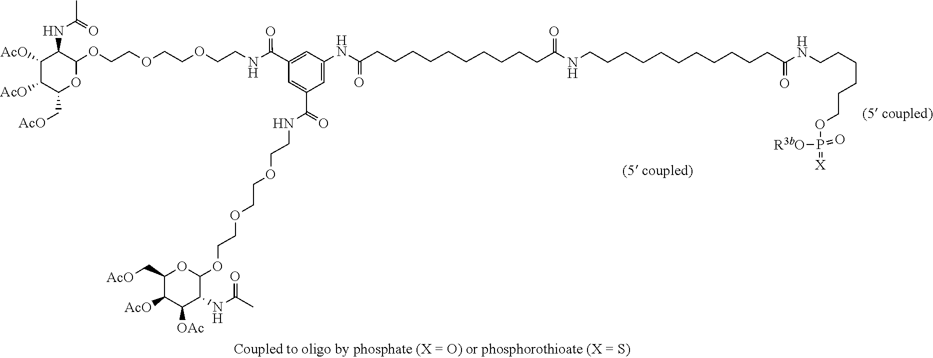

MS (+VE) calculated: 8254.0; measured: 8253.5

### Example 12: In Vivo Testing of TTR siRNA Bidentate Conjugates

Bidentate conjugates (Examples 1-8), wherein the oligonucleotide is the modified TTR siRNA described in Table A were tested for in vivo activity in a wild-type mouse model of TTR knock down. The TTR conjugates are a possible treatment for the orphan disease of TTR (Transthyretin) amyloidosis. In those afflicted with this disease the misfolding and aggregation of the Transthyretin protein is known to be associated with disease progression. By using this siRNA conjugate the amount of misfolded/aggregated protein in the patient can be reduced with a possible result of halting the progression of the disease.

TABLE A
Chemically Modified TTR siRNA duplexes

| | Sense | | Antisense | |
|---|---|---|---|---|
| siRNA | strand | Sense strand | strand | Antisense strand |
| Number | SEQ ID NO | 5′-3′ | SEQ ID NO | 5′-3′ |
| 1 | SEQ ID | AsasCaGuGuUCUuGcUcUaUaA | SEQ ID NO: | usUsaUaGaGcAagaAcAcUgUususu |
| | NO: 1 | | 2 | |
| 2 | SEQ ID | sAsaCaGuGuUCUuGcUcUaUsa | SEQ ID NO: | usUsaUaGaGcAagaAcAcUgUususu |
| | NO: 3 | sA | 4 | |

2′-O-Methyl nucleotides = lower case;
2′-Fluoro nucleotides = UPPER CASE;
Phosphorothioate linker = s;
Unmodified = UPPER CASE

Both the TTR siRNA sequence & animal model were described by Nair et al. J. Am. Chem. Soc., 2014, 136 (49), 16958-16961. All animal-related procedures were conducted according to written operating procedures, in accordance with Canadian Council on Animal Care (CCAC) Guidelines on Good Animal Practices, and approved by the local Institutional Animal Care and Use Committee (IACUC).

siRNA treatment: Female C57BL/6 mice (n=4) were administered a single 2 mg/kg dose of TTR siRNA conjugates once on Day 0 (1 dose per animal) via subcutaneous injection in the scapular region. One group of animals administered vehicle only (PBS) served as controls.

Collections: All animals were test bled at defined time points after test article administration (days 2, 4, 5, 7, 8, 9, 14 and 21) to determine maximum reductions in plasma TTR levels and the duration of pharmacologic activity.

Analysis: TTR protein levels in plasma samples were determined using the Abnova Prealbumin (Mouse) ELISA kit (Cedar Lane, catalogue number KA2070) as per the manufacturer's instructions. TTR plasma protein values were calculated for the individual plasma samples and the average of each group was determined. From these averages, the TTR protein levels relative to control (% relative to PBS treated animals) were determined.

Results: Results from testing are presented in Table B. Values represent % TTR protein levels (relative to PBS Control) on Days 2, 4, 5, 7, 8, 9, 14 & 21 post treatment.

Conclusion: Animals treated with TTR bidentate conjugates exhibited a marked knockdown of target mRNA and protein with maximal knock down of TTR protein occurring between days 4 & 9 post subcutaneous injection.

TABLE B
Plasma TTR protein levels in mice after single IV administration (2 mg/kg) of
GalNAc bidentate conjugated siRNA from Table A.
TTR protein data expressed as percent of PBS treated mouse values

| | siRNA | Day | Day | Day | Day | Day | Day | Day | Day |
|---|---|---|---|---|---|---|---|---|---|
| Example | Number | 2 | 4 | 5 | 7 | 8 | 9 | 14 | 21 |
| 1 | 1 | 27.1 | 11.2 | — | 10.8 | — | 11.9 | 22.2 | 50.9 |
| 2 | 1 | 31.9 | 14.2 | — | 12.7 | — | 14.6 | 35.0 | 53.9 |
| 3 | 1 | 31.0 | 15.0 | — | 17.1 | — | 26.8 | 34.0 | 57.2 |
| 4 | 1 | 31.0 | 12.4 | — | 15.5 | — | 17.0 | 35.2 | 49.3 |
| 5 | 1 | 28.5 | 13.3 | — | 13.1 | — | 20.1 | 31.3 | 49.7 |
| 6 | 1 | 26.9 | 8.3 | — | 11.6 | — | — | 20.3 | 49.8 |
| 7 | 1 | 33.2 | 11.3 | — | 15.1 | — | — | 25.9 | 63.0 |
| 8 | 1 | 20.3 | — | 7.3 | — | 8.4 | — | 14.4 | 37.2 |
| 9 | 1 | 29.0 | — | 9.7 | — | 9.4 | — | 19.1 | 40.8 |
| 10 | 2 | 16.9 | — | 6.7 | — | 7.1 | — | 8.7 | 19.9 |
| 11 | 2 | 21.6 | — | 7.2 | — | 9.6 | — | 11.7 | 23.4 |

### Example 13: In Vivo Testing of TTR siRNA Bidentate Conjugates

Monovalent, bidentate, trivalent & tetravalent conjugates (Compounds A-D), wherein R^{3b} is the modified TTR siRNA described in Table A above, were tested for in vivo activity in a wild-type mouse model of TTR knock down.

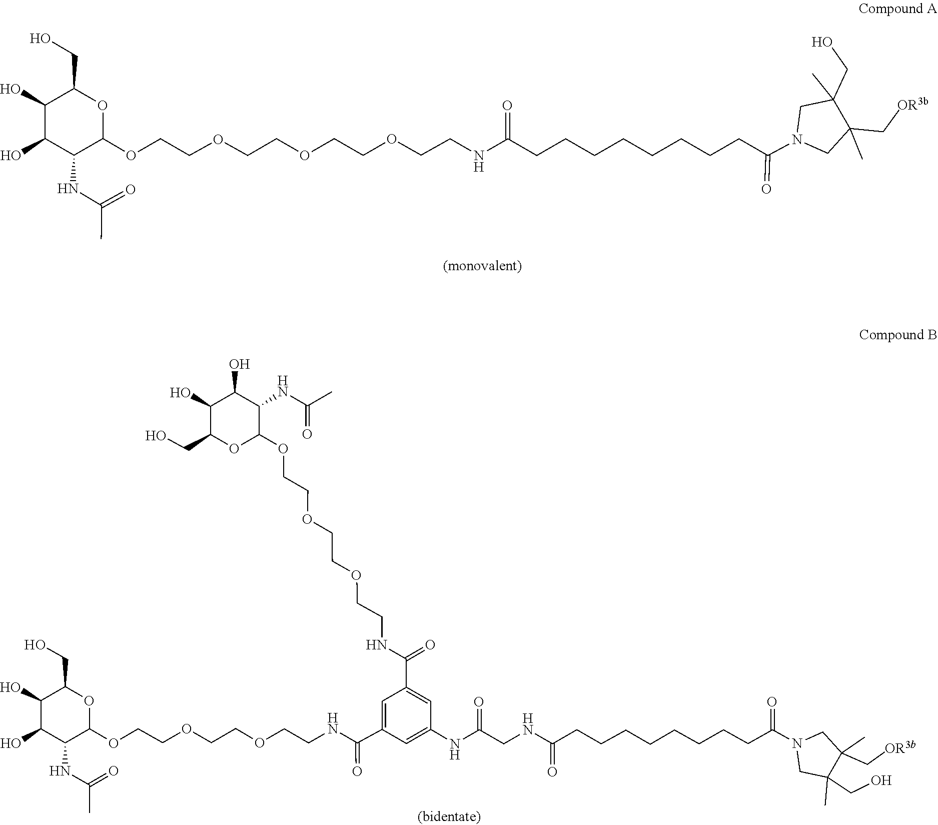

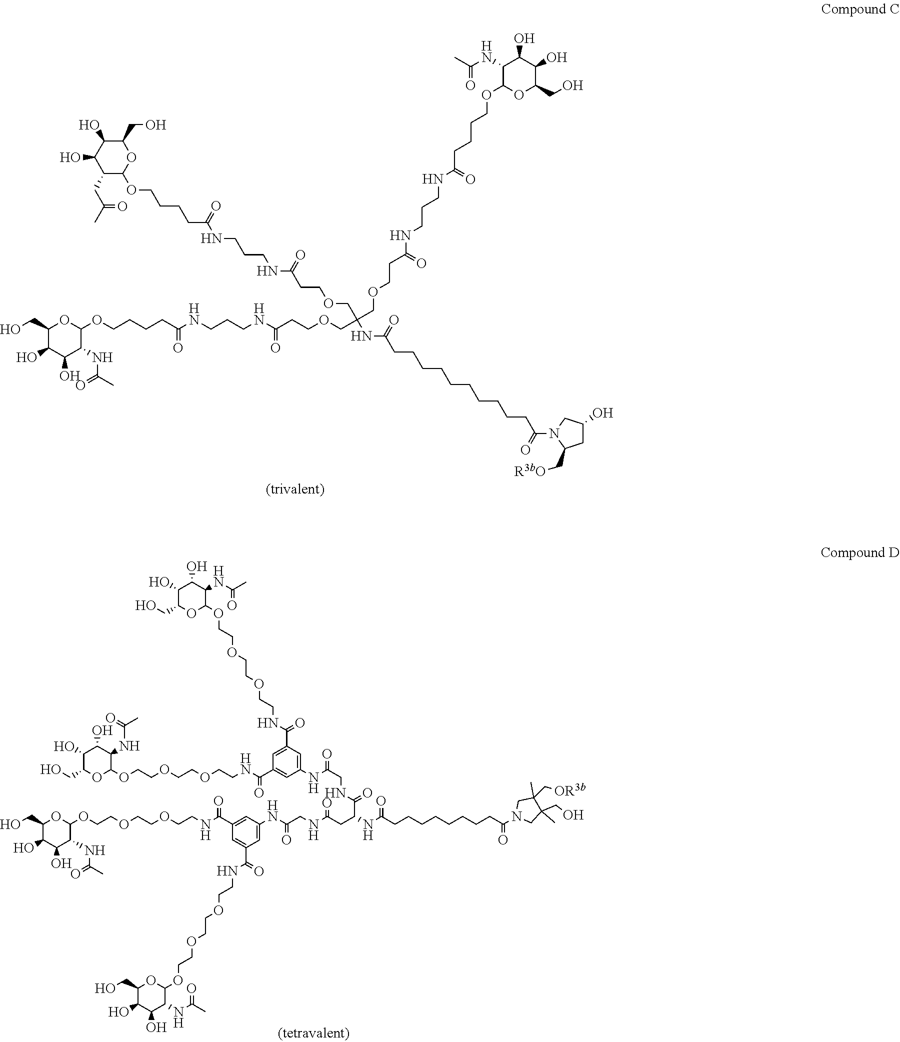

The TTR conjugates are a possible treatment for the orphan disease of TTR (Transthyretin) amyloidosis. In those afflicted with this disease the misfolding and aggregation of the Transthyretin protein is known to be associated with disease progression. By using this siRNA conjugate the amount of misfolded/aggregated protein in the patient can be reduced with a possible result of halting the progression of the disease.

Both the TTR siRNA sequence & animal model were described by Nair et al. J. Am. Chem. Soc., 2014, 136 (49), 16958-16961. All animal-related procedures were conducted according to written operating procedures, in accordance with Canadian Council on Animal Care (CCAC) Guidelines on Good Animal Practices, and approved by the local Institutional Animal Care and Use Committee (IACUC).

siRNA treatment: Female C57BL/6 mice (n=4) were administered a single 2 mg/kg dose of TTR siRNA conjugates once on Day 0 (1 dose per animal) via subcutaneous injection in the scapular region. One group of animals administered vehicle only (PBS) served as controls.

Collections: All animals were test bled at defined time points after test article administration (days 2, 5, 7, 14 and 21) to determine maximum reductions in plasma TTR levels and the duration of pharmacologic activity.

Analysis: TTR protein levels in plasma samples were determined using the Abnova Prealbumin (Mouse) ELISA kit (Cedar Lane, catalogue number KA2070) as per the manufacturer's instructions. TTR plasma protein values were calculated for the individual plasma samples and the average of each group was determined. From these averages, the TTR protein levels relative to control (% relative to PBS treated animals) were determined.

Results: Results from testing are presented in Table C. Values represent % TTR protein levels (relative to PBS Control) on Days 2, 5, 7, 14 & 21 post treatment.

Conclusion: Animals treated with TTR bidentate, trivalent & tetravalent conjugates exhibited similar levels of knockdown of target mRNA and protein with maximal knock down of TTR protein occurring between days 2 & 7 post subcutaneous injection. The TTR monovalent conjugate showed very little, if any, knockdown of target mRNA and protein.

TABLE C
Plasma TTR protein levels in mice after single IV administration (2
mg/kg) of GalNAc conjugated siRNA Compounds A-D.
TTR protein data expressed as percent of PBS treated mouse values

| | siRNA | Day | Day | Day | Day | Day |
|---|---|---|---|---|---|---|
| Compound | Number | 2 | 5 | 7 | 14 | 21 |
| A | 1 | 89.4 | 79.6 | 82.7 | 71.0 | 107.7 |
| B | 1 | 26.9 | 10.1 | 11.5 | 25.9 | 57.5 |
| C | 1 | 20.6 | 11.6 | 12.7 | 26.9 | 48.6 |
| D | 1 | 29.3 | 10.1 | 11.6 | 23.2 | 47.9 |

1. A conjugate of formula (I): 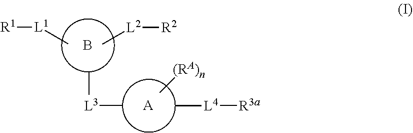
wherein:
R¹ is a saccharide;
L¹ is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
B is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)allyl;
L² is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
R² is a saccharide;
L³ is absent or a linking group;
A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
n is 0, 1, 2, 3, 4, 5, 6, 7, 8, 9, or 10;
L⁴ is absent or a linking group;
R³ is a nucleic acid;
R^{a} is hydrogen, a protecting group, a covalent bond to a solid support, or a bond to a linking group L⁵ that is bound to a solid support; and
L⁵ is a linking group;
or a salt thereof.

2. The conjugate or salt of claim 1 wherein A is absent.

3. The conjugate or salt of claim 1 wherein A is a 3-20 membered cycloalkyl, a 5-20 membered aryl, a 5-20 membered heteroaryl, or a 3-20 membered heterocycloalkyl.

4. The conjugate or salt of claim 1, wherein B is a 5-10 membered aryl.

5. The conjugate or salt of claim 1, wherein B is naphthyl or phenyl.

6. The conjugate or salt of claim 1, wherein B is phenyl.

7. The conjugate or salt of claim 1, wherein the group: 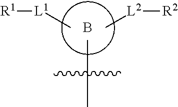
is:

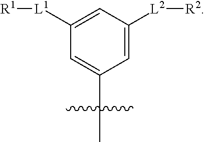

8. The conjugate or salt of claim 1, wherein B is a 5-10 membered heteroaryl.

9. The conjugate or salt of claim 1, wherein B is pyridyl, pyrimidyl, quinolyl, isoquinolyl, imidazoyl, thiazolyl, dioxazolyl or oxazolyl.

10. The conjugate or salt of claim 1, wherein the group: 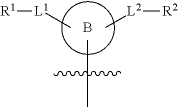
is:

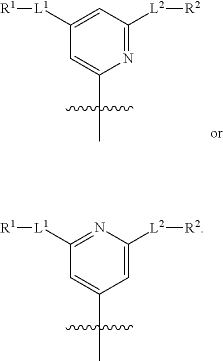

11. The conjugate or salt of claim 1, wherein the group: 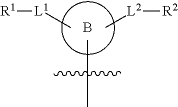
is:

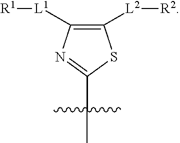

12. The conjugate or salt of claim 1, wherein L¹ is a divalent, unbranched, saturated hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

13. The conjugate or salt of claim 1, wherein L¹ is a divalent, unbranched, saturated hydrocarbon chain, having from 0 to 12 carbon atoms, wherein one or more of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

14. The conjugate or salt of claim 1, wherein L¹ is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

15. The conjugate or salt of claim 1, wherein L² is a divalent, unbranched, saturated hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

16. The conjugate or salt of claim 1, wherein L² is a divalent, unbranched, saturated hydrocarbon chain, having from 0 to 12 carbon atoms, wherein one or more of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

17. The conjugate or salt of claim 1, wherein L² is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

18. The conjugate or salt of claim 1, wherein R¹ is: 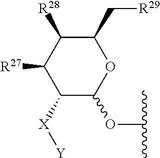
wherein:
X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;
each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

19. The conjugate or salt of claim 1, wherein R¹ is:

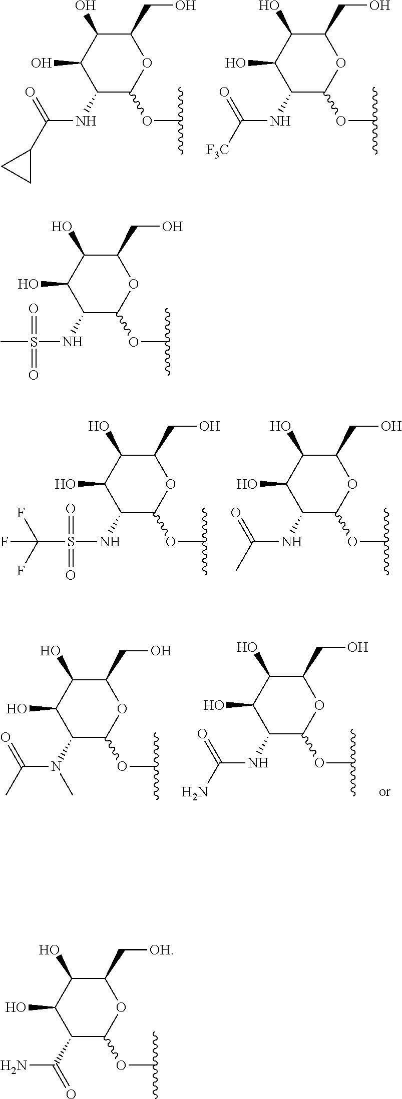

20. The conjugate or salt of claim 1, wherein R¹ is:

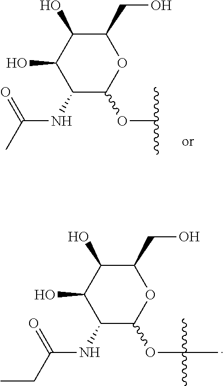 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

21. The conjugate or salt of claim 1, wherein R¹ is:

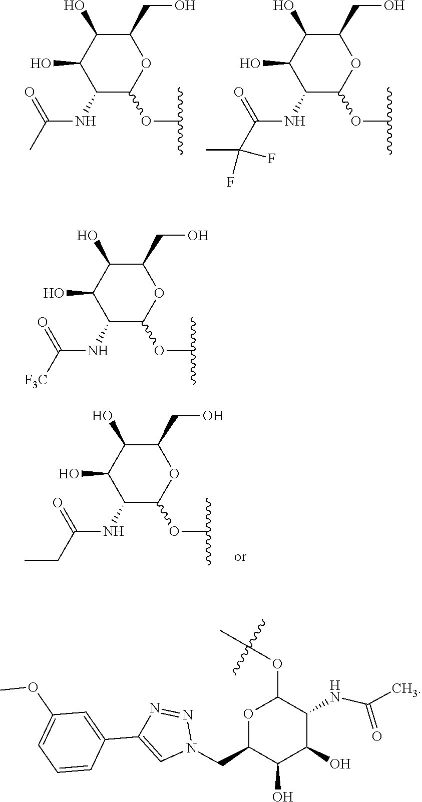

22. The conjugate or salt of claim 1, wherein R¹ is:

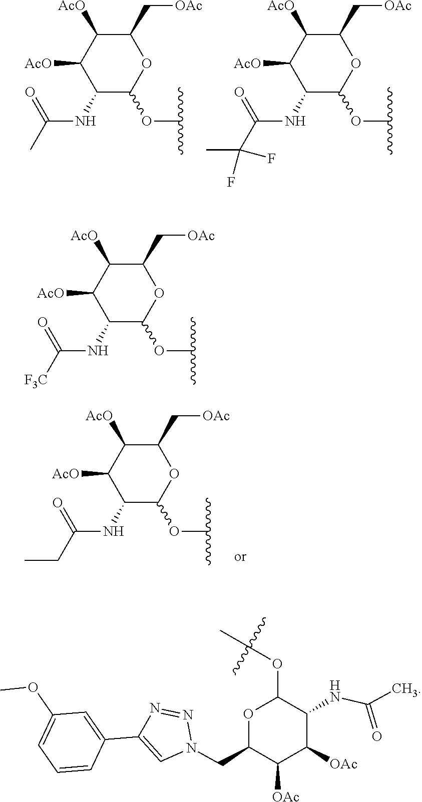

23. The conjugate or salt of claim 1, wherein R² is: 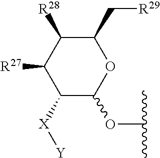
wherein:
X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;
each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

24. The conjugate or salt of claim 1, wherein R² is:

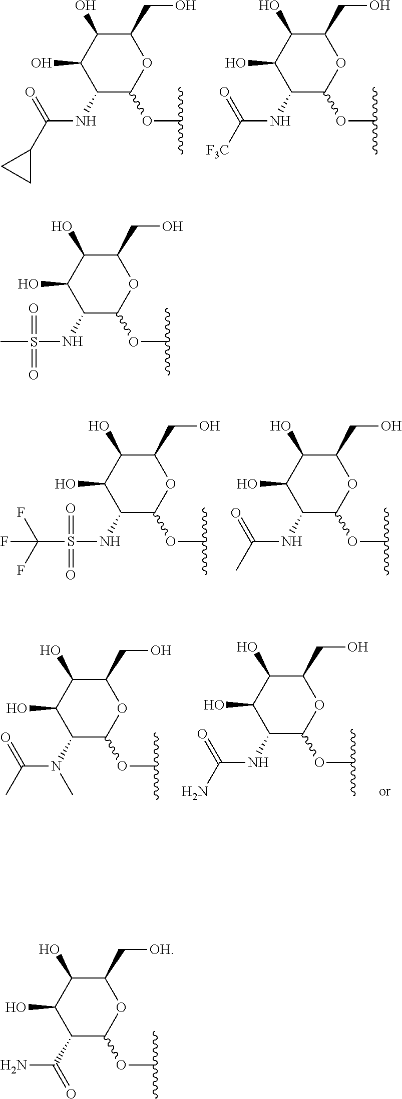

25. The conjugate or salt of claim 1, wherein R² is:

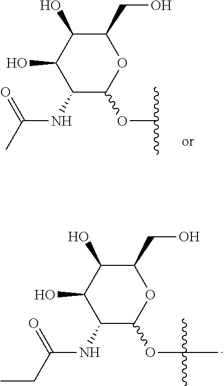 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

26. The conjugate or salt of claim 1, wherein R² is:

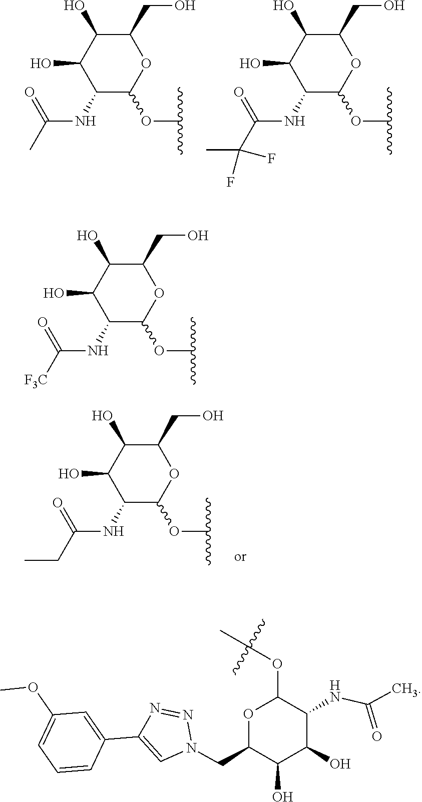

27. The conjugate or salt of claim 1, wherein R² is:

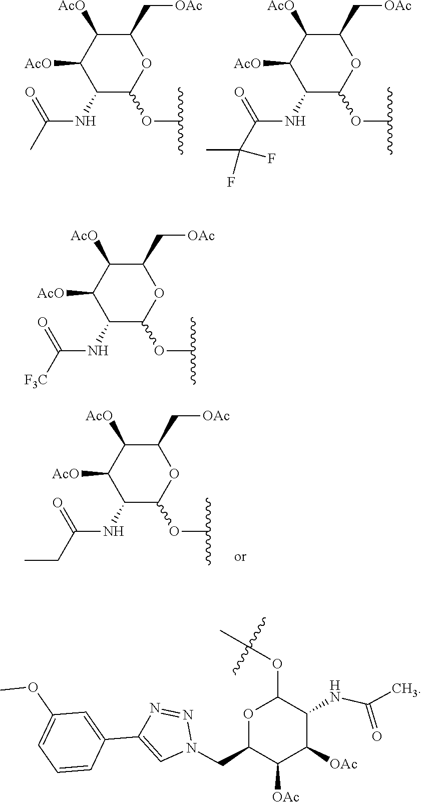

28. The conjugate or salt of claim 1, wherein L³ is a divalent, branched or unbranched, saturated or unsaturated, hydrocarbon chain, having from 0 to 50 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

29. The conjugate or salt of claim 1, wherein L³ is a divalent, branched or unbranched, saturated or unsaturated, hydrocarbon chain, having from 1 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

30. The conjugate or salt of claim 1, wherein L³ is a divalent, branched or unbranched, saturated or unsaturated, hydrocarbon chain, having from 1 to 30 carbon atoms, wherein one or more of the carbon atoms is optionally replaced by —O—, —NR^{X}— —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

31. The conjugate or salt of claim 1, wherein L³ is:

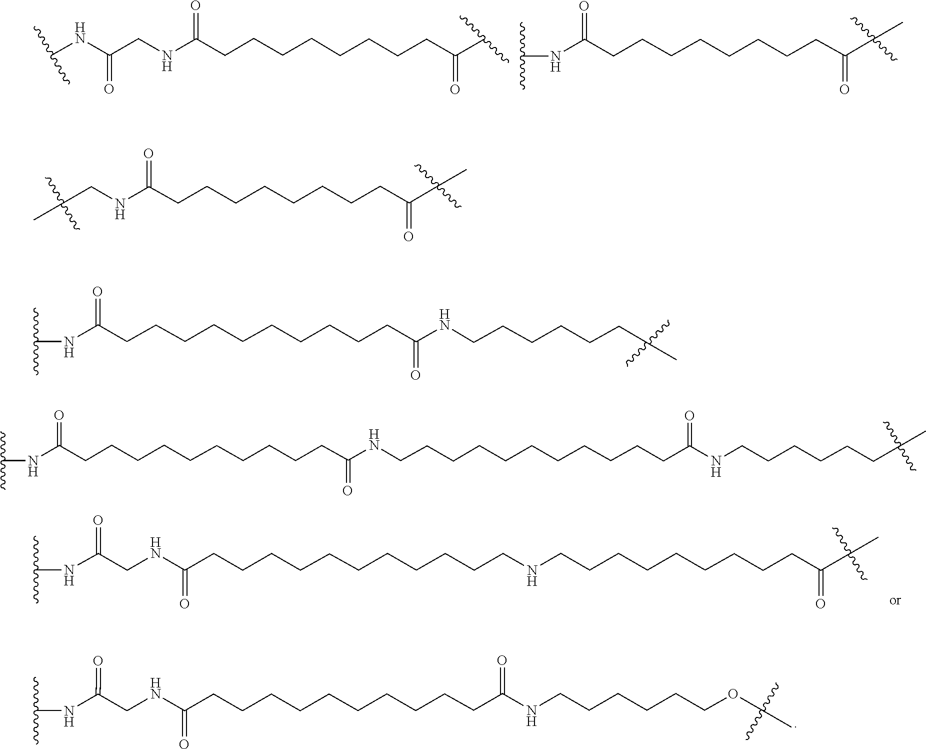

32. The conjugate or salt of claim 1, wherein L³ is connected to B through —NH—, —O—, —S—, —(C═O)—, —(C═O)—NH—, —NH—(C═O)—, —(C═O)—O—, —NH—(C═O)—NH—, or —NH—(SO₂)—.

33. The conjugate or salt of claim 1, wherein L⁴ is a divalent, branched or unbranched, saturated or unsaturated, hydrocarbon chain, having from 0 to 50 carbon atoms, wherein one or more of the carbon atoms in the hydrocarbon chain is optionally replaced by —O—, —NR^{X}—, —NR^{X}—C(═O)—, —C(═O)—NR^{X}— or —S— and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

34. The conjugate or salt of claim 1, wherein L⁴ is a divalent, branched or unbranched, saturated or unsaturated, hydrocarbon chain, having from 1 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

35. The conjugate or salt of claim 1, wherein L⁴ is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

36. The conjugate or salt of claim 1, wherein L⁴ is connected to R³ through —O—.

37. The conjugate or salt of claim 1, wherein the group: 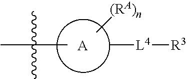
is selected from the group consisting of: 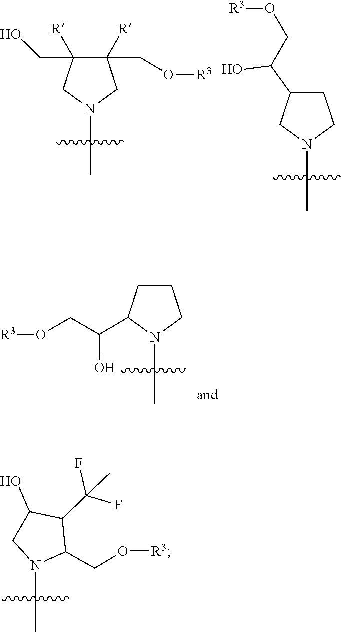
wherein
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

38. The conjugate or salt of claim 1, wherein the group: 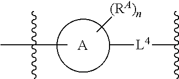
is selected from the group consisting of: 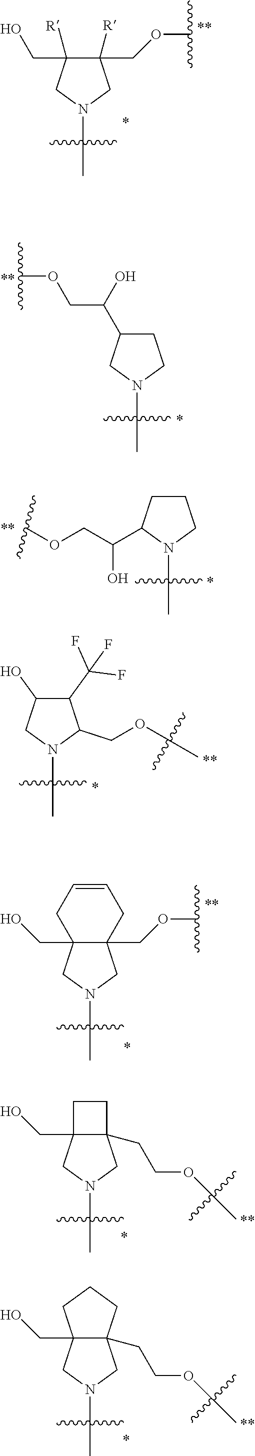 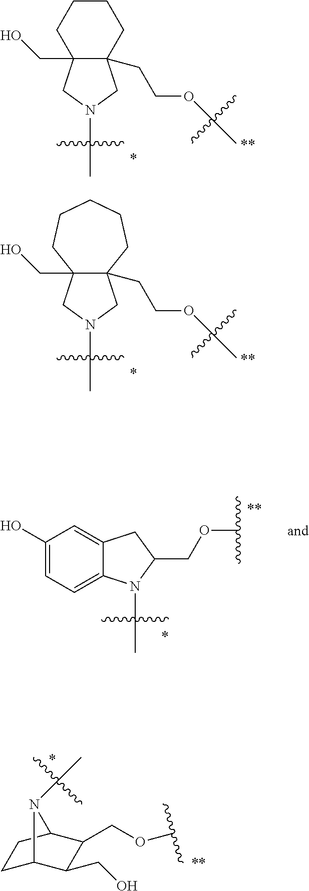
wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L³; and
the valence marked with ** is attached to R³.

39. The conjugate or salt of claim 1, wherein the group: 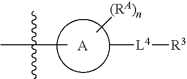
is:

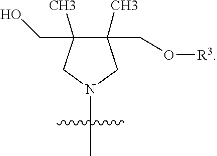

40. A conjugate selected from the group consisting of: 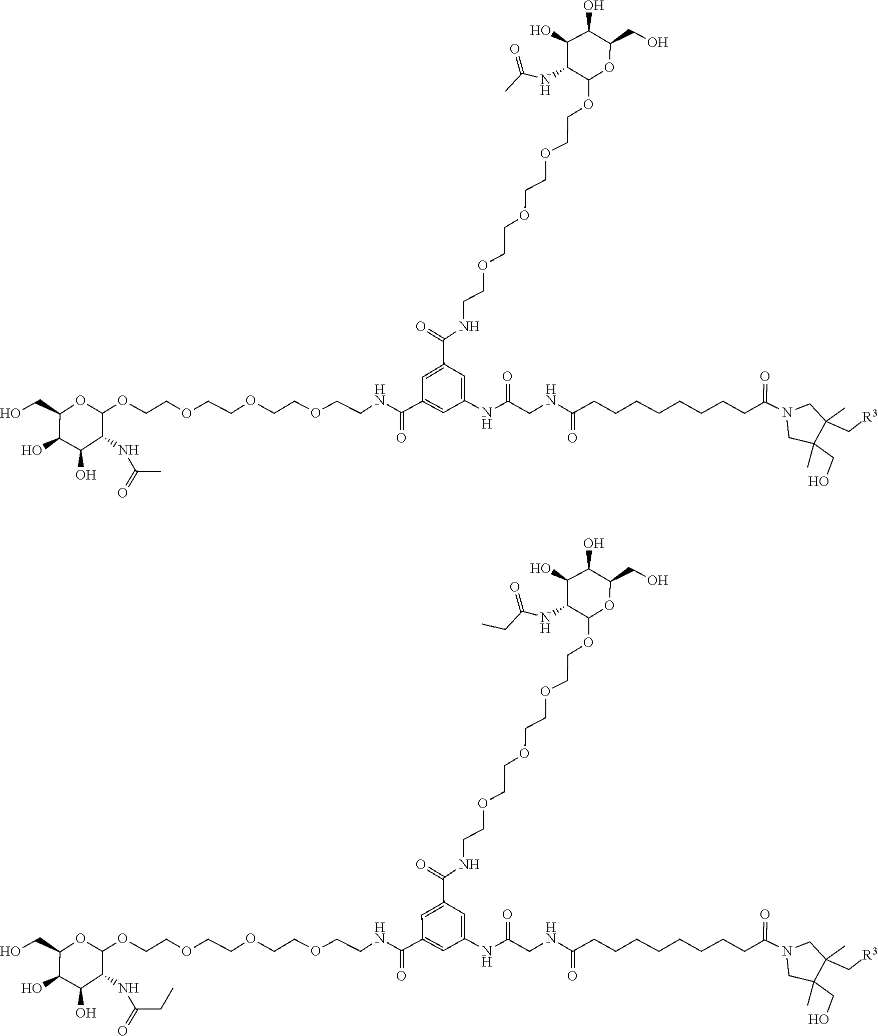 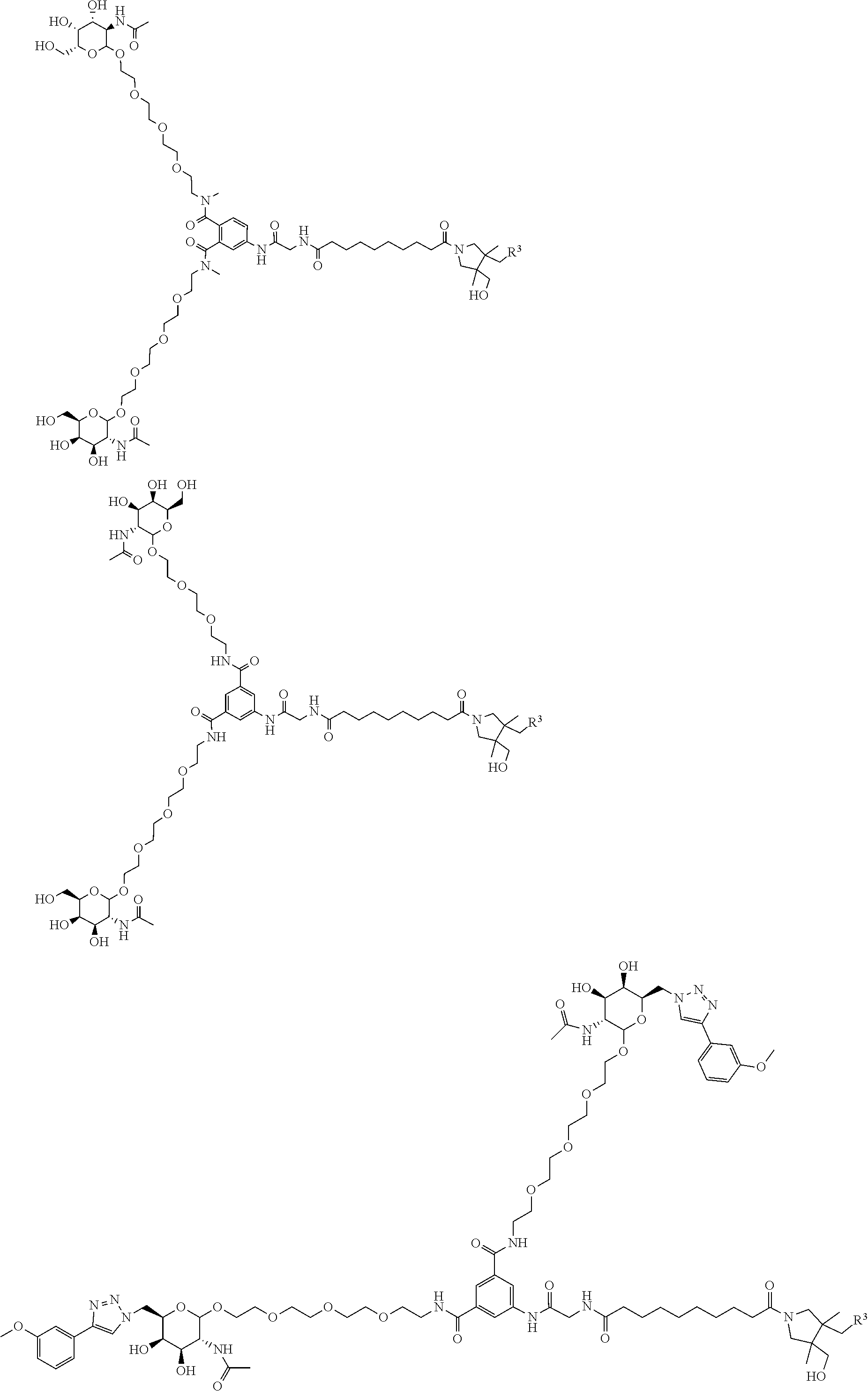 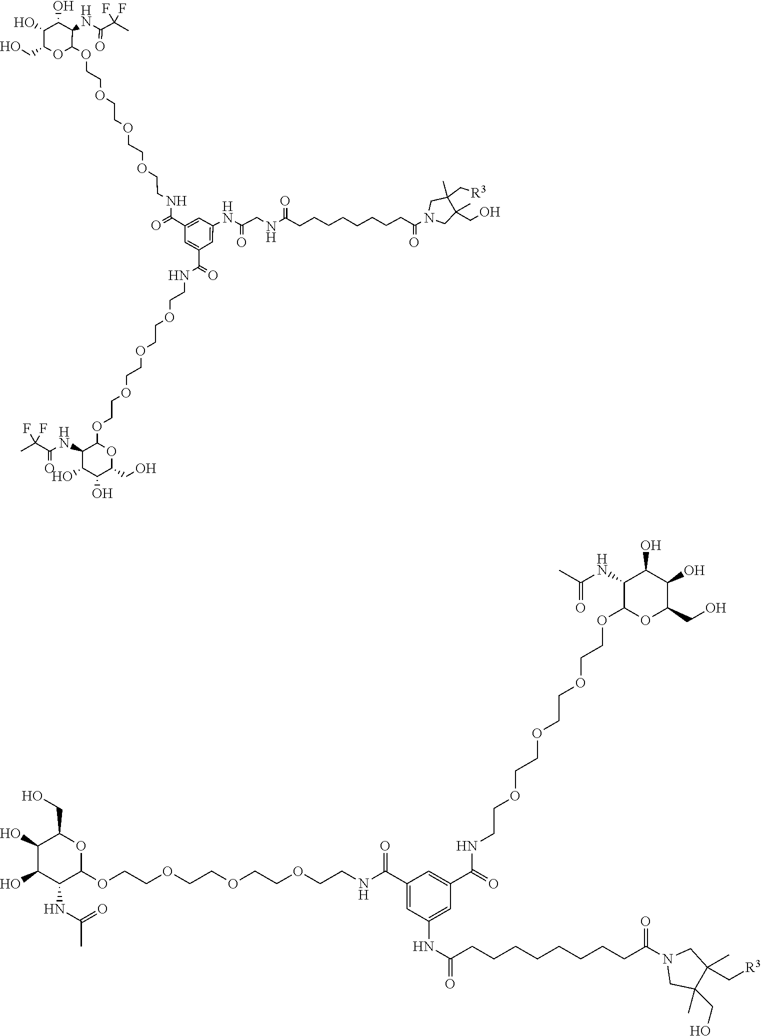 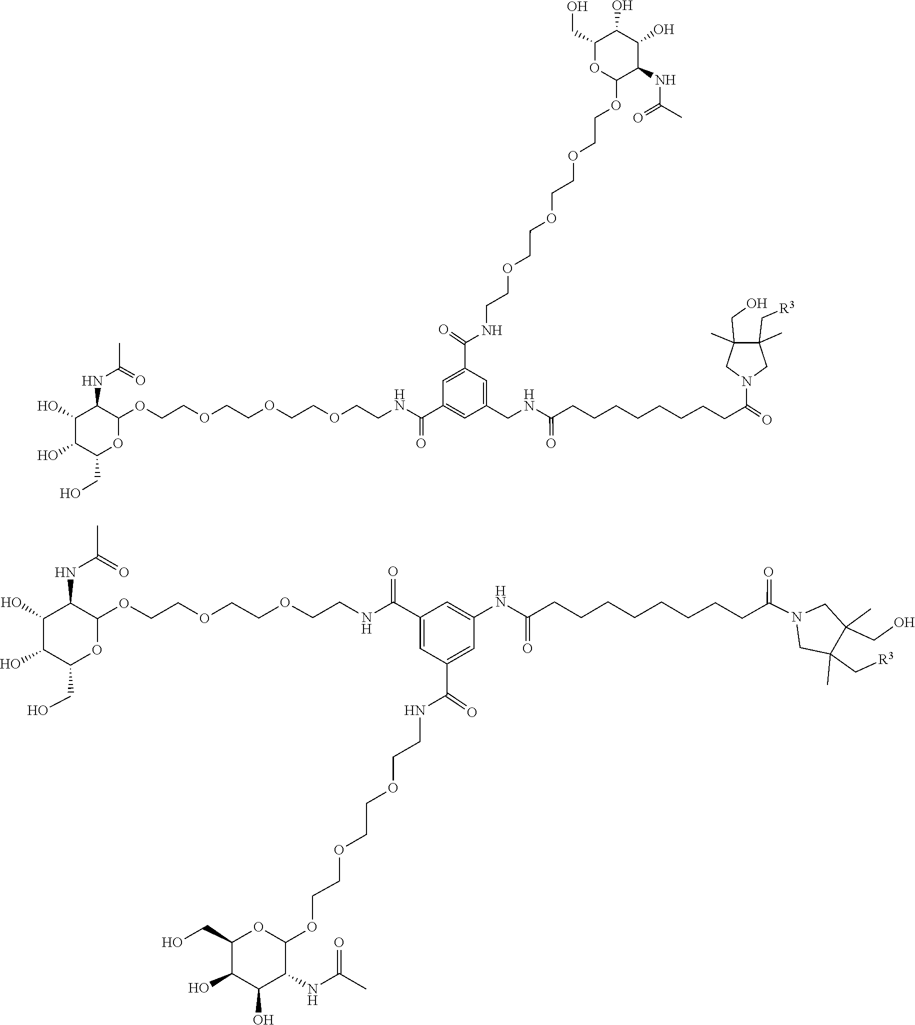 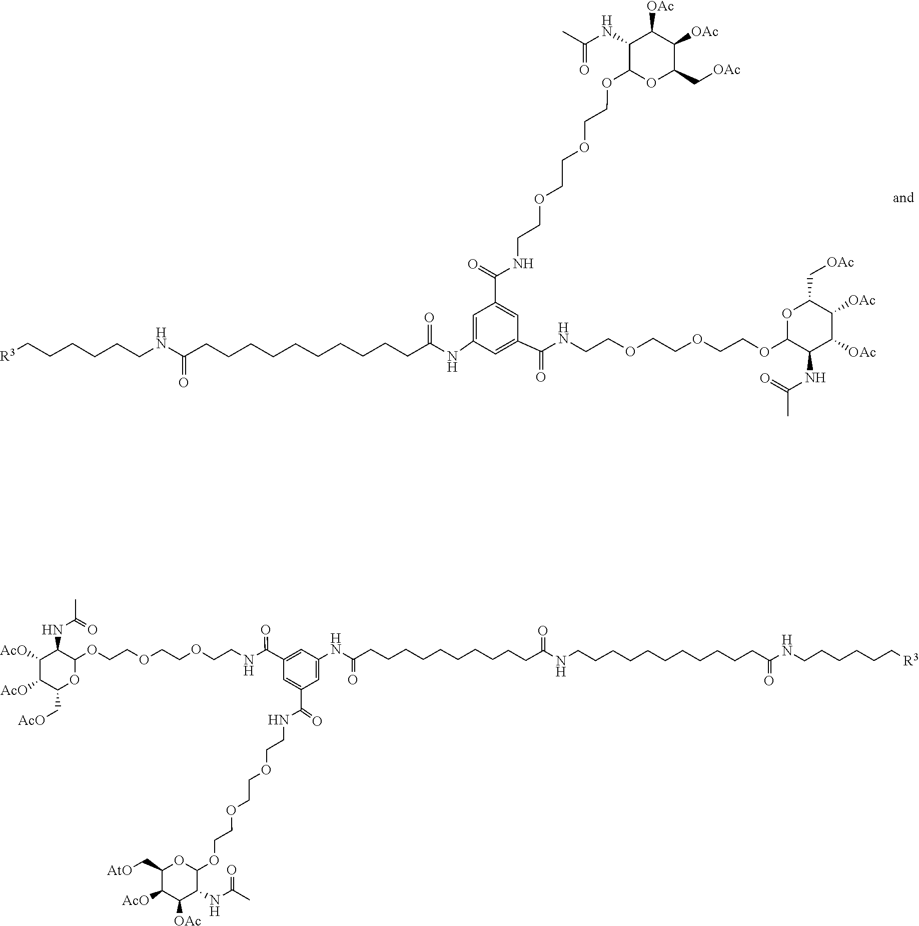
wherein: R³ is a nucleic acid; or a salt thereof.

41. A pharmaceutical composition comprising a conjugate described in claim 1, or a pharmaceutically acceptable salt thereof, and a pharmaceutically acceptable carrier.

42. A compound of formula (Ia): 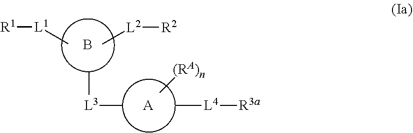
wherein:
R¹ a is a saccharide;
L¹ is a divalent, branched or unbranched saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
B is a 5-10 membered aryl or a 5-10 membered heteroaryl, which 5-10 membered aryl or a 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl;
L² is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
R² a is a saccharide;
L³ is absent or a linking group;
A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
n is 0, 1, 2, 3, 4, 5, 6, 7, 8, 9, or 10;
L⁴ is absent or a linking group;
R^{3a} is H, a protecting group, a synthetic activating group, a covalent bond to a solid support, or a bond to a linking group that is bound to a solid support;
R^{a} is hydrogen, a protecting group, a covalent bond to a solid support, or a bond to a linking group L⁵ that is bound to a solid support; and
L⁵ is a linking group;
or a salt thereof.

43-52. (canceled)

53. A compound selected from the group consisting of: 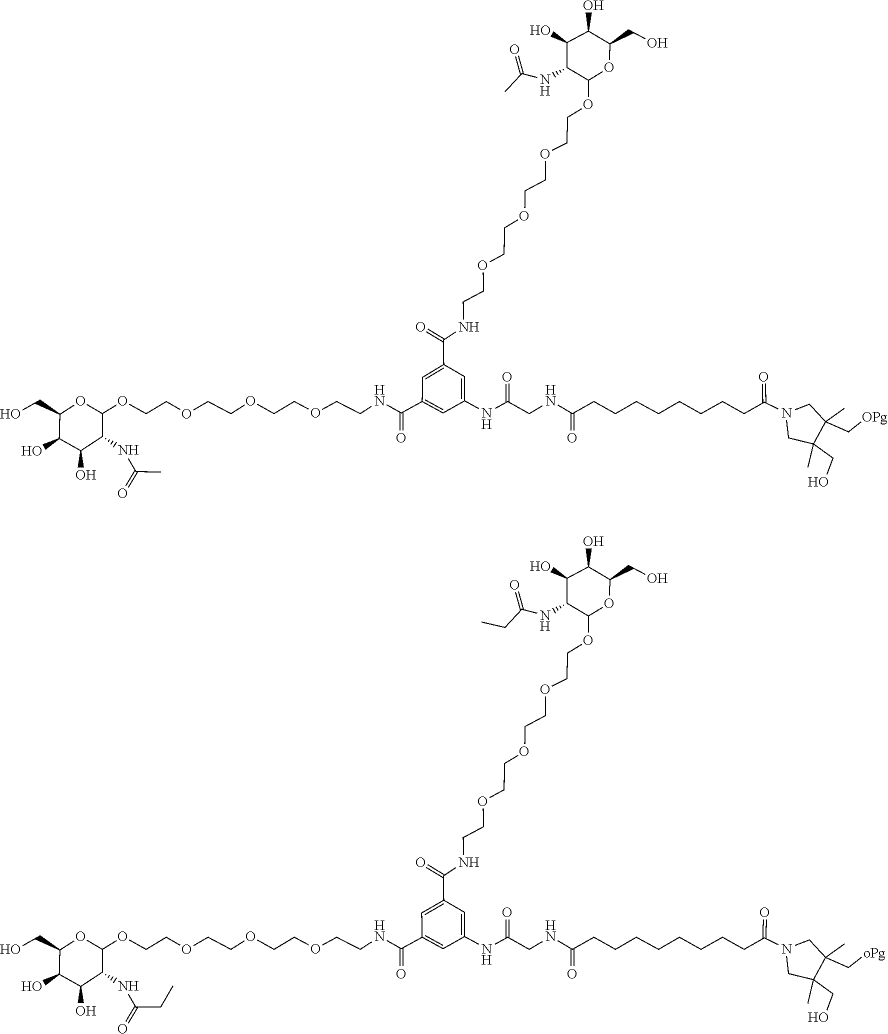 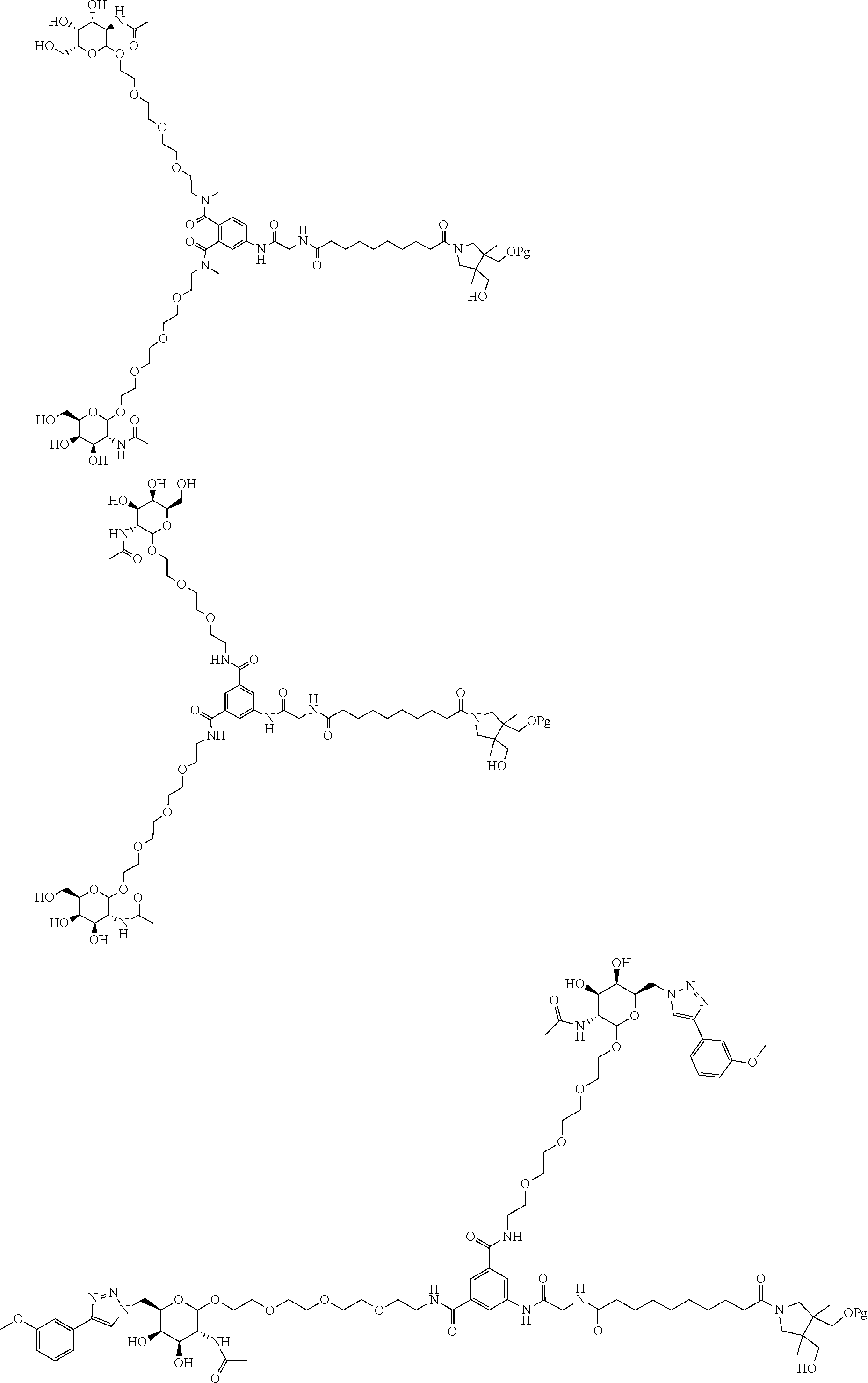 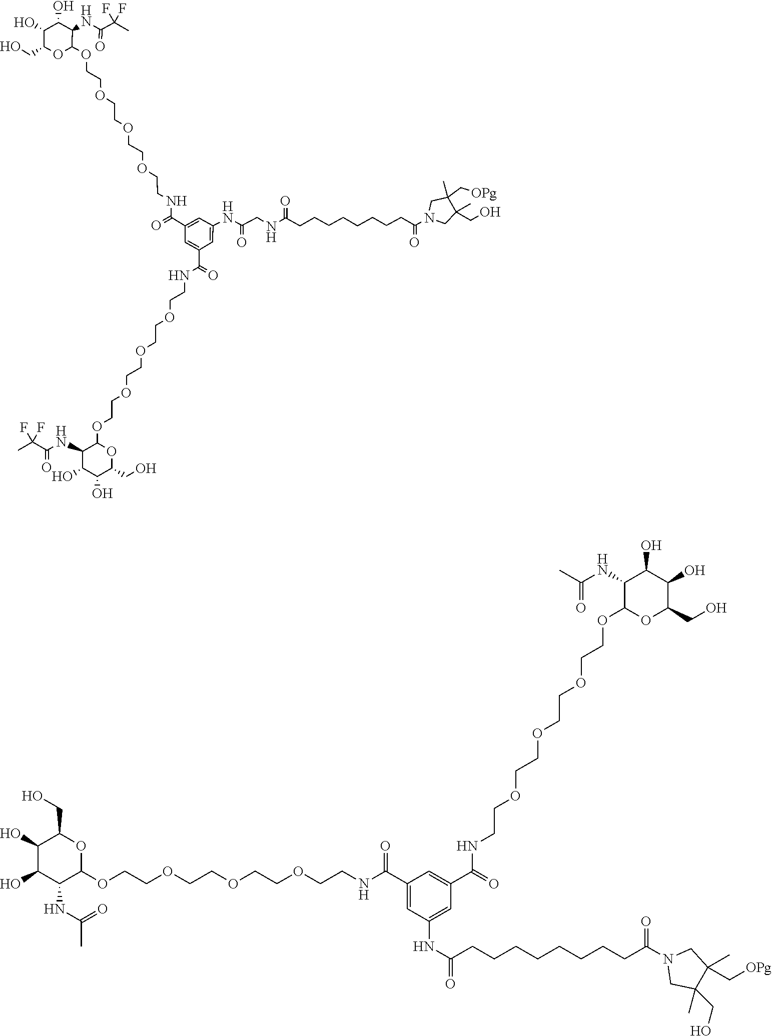 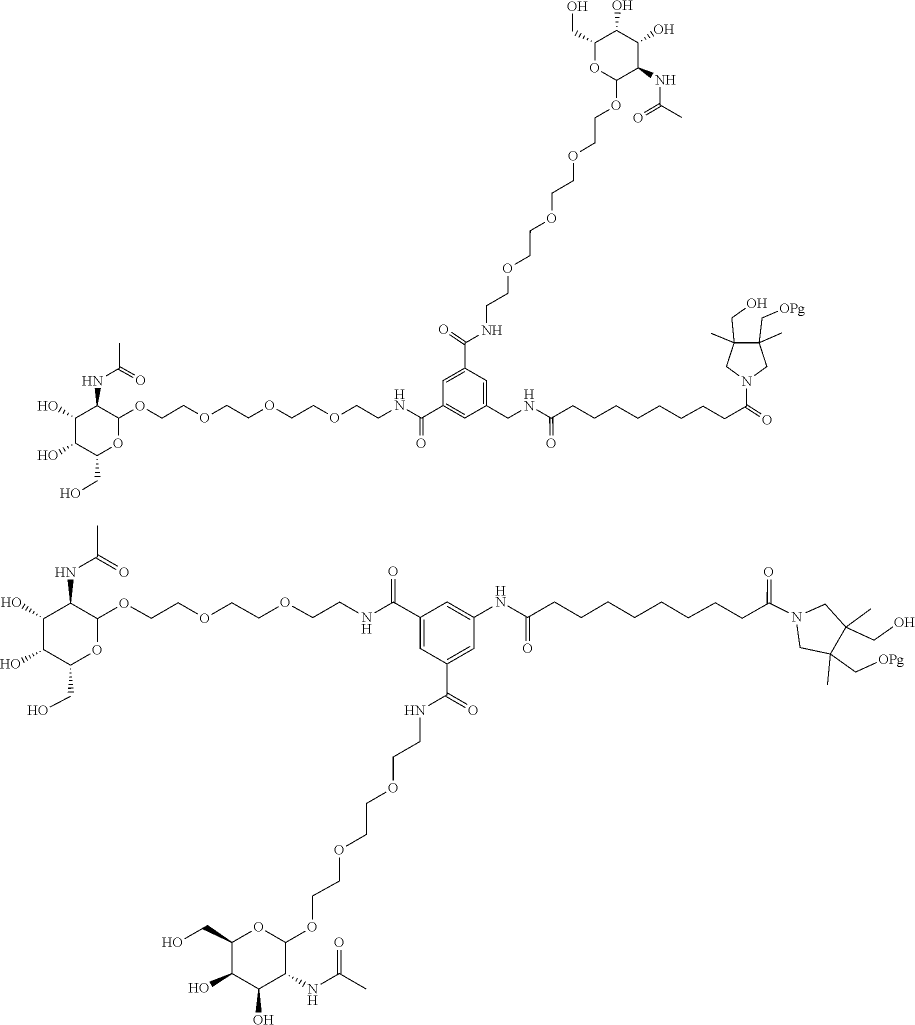 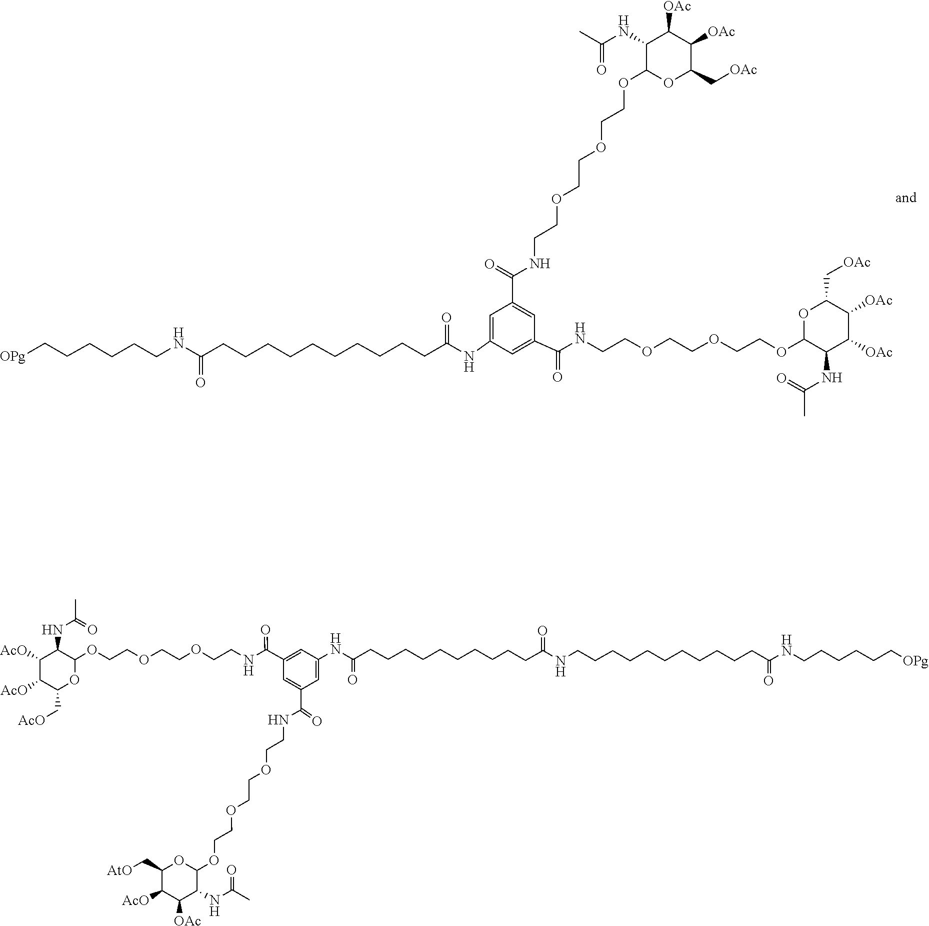
wherein Pg is a protecting group; or a salt thereof.

54. The conjugate or salt of claim 1, wherein R³ is a siRNA.
